(19) United States
(12) Patent Application Publication
(10) Pub. No.: US 2007/0014797 A1
(43) Pub. Date: Jan. 18, 2007

(54) Method for treating Sjogren's syndrome

(75) Inventor: Elena Hitraya, Foster City, CA (US)

Correspondence Address: GENENTECH, INC., 1 DNA WAY, SOUTH SAN FRANCISCO, CA, 94080, US

(73) Assignee: Genentech, Inc., South San Francisco, CA (US)

(21) Appl. No.: 11/524,231

(22) Filed: Sep. 19, 2006

Related U.S. Application Data
(63) Continuation of application No. 11/187,364, filed on Jul. 21, 2005.
(60) Provisional application No. 60/590,302, filed on Jul. 22, 2004.

(51) Int. Cl. A61K 39/395; A61K 31/66; A61K 31/60; A61K 31/525; A61K 31/522; A61K 31/573; A61K 31/4178; A61K 31/405; A61K 31/4706; A61K 31/192

(52) U.S. Cl. 424/144.1; 514/11; 514/313; 514/171; 514/397; 514/165; 514/569; 514/570; 514/263.31; 514/251; 514/109; 514/420

(57) ABSTRACT

A method of treating Sjögren's syndrome in a patient eligible for treatment is provided involving administering an effective amount of an antagonist that binds to a B-cell surface marker to the patient to provide significant improvement over baseline in two or more of dryness, fatigue, and joint pain on a Visual Analogue Scale, and an article of manufacture therefor. Methods and articles are also provided involving treating Sjögren's syndrome in a subject eligible for treatment is provided involving administering an effective amount of an antibody that binds to a B-cell surface marker to the subject to provide an initial exposure and a subsequent exposure to the antibody within certain dosing regimens and an article of manufacture therefor.

### RELATED APPLICATIONS

This application is a continuation of U.S. Ser. No. 11/187,364 filed Jul. 21, 2005, which claims priority under 35 U.S.C. § 119(e) to U.S. Ser. No. 60/590,302, filed Jul. 22, 2004 the contents of which are incorporated herein by reference.

### FIELD OF THE INVENTION

The present invention concerns methods for treating Sjögren's syndrome in a subject, and kits with instructions for such use.

### BACKGROUND OF THE INVENTION

### Sjögren's Syndrome

Autoimmune diseases, such as Sjögren's syndrome and lupus, among others, remain clinically important diseases in humans. As the name implies, autoimmune diseases wreak their havoc through the body's own immune system. While the pathological mechanisms differ between individual types of autoimmune diseases, one general mechanism involves the binding of certain antibodies (referred to herein as self-reactive antibodies or autoantibodies) present.

Sjögren's syndrome is a chronic disorder in which white blood cells attack the moisture-producing glands. The hallmark symptoms are dry eyes and dry mouth, caused by lymphocytic infiltrates of lacrimal and salivary glands. The loss of tears and saliva may result in characteristic changes in the eyes (called aqueous tear deficiency or keratoconjunctivitis sicca) and in the mouth with deterioration of the teeth, increased oral infection, difficulty in swallowing, and painful mouth. Patients may also have inflammation of the joints (arthritis), muscles (myositis), nerves (neuropathy), thyroid (thyroiditis), kidneys (nephritis), lungs, or other areas of the body, or lymph node swelling. Also, patients may experience fatigue and sleep disruption. It is one of the most prevalent autoimmune disorders, striking as many as four million Americans, mainly middle-aged women.

The American-European classification criteria for Sjögren's syndrome are set forth in Vitali et al, Ann Rheum Dis 61: 554-558 (2002). Currently, treatments are symptomatic; there is a need for treatment based on pathogenic data. The major complaints in primary Sjögren's syndrome are sicca symptoms (mouth dryness and eye dryness), fatigue, arthralgia/-itis, and systemic involvement (heterogenous). See, e.g., Hay et al., Brit J Rheum, 37 (10): 1069-1076 (1998). Dryness is the dominant complaint, and there is a weak association between subjective symptoms and objective testing (Hay et al., Ann Rheum Dis, 57 (1): 20-24 (1998)). No gold standard exists for symptoms assessment. The objective measures (USF, Shirmer) assess the severity of glandular impairment and not the degree of dyscomfort/dysfunction. The primary endpoints may include improvement across two out of four Sjögren's disease domains: ocular dryness, oral dryness, fatigue, and laboratory tests. There may be ocular improvement that could be ≧20% improvement in: patients' assessment of dry eyes (Visual Analogue Scale (VAS)), Shirmer I test (with/without anesthesia) such as 0-25 mm of wetting in 5 min for each eyes, and an ocular dye test scored according to van Bijsterveld. Secondary endpoints may include 0-9 for each eye, following lissamine green staining, or there may be oral improvement that is ≧20% improvement in: patients' assessment of dry mouth, unstimulated salivary flow (collected for 15 min using the spitting technique (Navazesh, Ann N Y Acad Sci, 694: 72-77(1993), with samples weighted on an analytical balance (1 g=1 ml)), there may be fatigue improvement that is ≧20% improvement in: patients assessment of fatigue (to what degree have you experienced fatigue? How severe is the fatigue which you have been experienced?<<not at all>> (0 mm)-<<very severe>> (100 mm)); MFI (Smets et al., Psychosom Res 39:315 (1995)); MAF; and Sjögren's-based psychometric questionnaire (Bowman et al., Rheumatology 43 (6): 758-764 (2004)); Laboratory tests improvement may be ≧20% improvement in: ESR (mm/h), serum IgG (mg/dl). Other endpoints are fatigue (Sjögren's-based psychometric questionnaire), dry eyes, ocular dye test scored according to van Bijsterveld (0-9 for each eye, following lissamine green staining), use of artificial tears (number of times per day they used ophtalmic solutions), arthralgia, general (patient's global assessment (VAS 0-100 mm), pain (VAS 0-100 mm)), parotid/salivary gland enlargement, laboratory tests (RF, ANA, C′4, cryoglobulinemia), and Liverpool sicca index (Field et al., J Oral Pathol Med, 32 (3): 154-162 (2003)) (oral symptom domain, oral symptom control domain, sensory domain, ocular domain, and sexual function domain).

The use of infliximab in active primary Sjögren's syndrome was studied by Steinfeld et al, Arthritis Rheum, 44: 2371-2375 (2001). In this open-label study of a loading dose regimen of 3 infusions of infliximab in patients with active primary Sjögren's syndrome, there was a fast and significant improvement of all measures of disease activity, without major adverse experiences.

In a one-year follow-up study of infliximab in patients with active primary Sjögren's syndrome (Steinfeld et al., Arthritis Rheum, 46:3301-3303 (2002)), the significant improvement of disease manifestations was maintained over a one-year period. There was no loss of efficacy observed after re-treatment, no major adverse event, increasing episodes of infusion reactions, extension protocol of the 3-month pilot study, induction regimen of three infusions of infliximab (3 mg/kg) at weeks 0, 2, 6, maintenance regimen every 12 weeks over one year, and 20 weeks between re-infusion. Steinfeld et al., Arthritis Rheum, 46:2249-2251 (2002) states that infliximab restores proper AQP-5 distribution in Sjögren's syndrome patients.

Martin et al., Clin Exp Rheumatol, 21:412 (2003) disclosed use of infliximab in secondary Sjögren's syndrome in rheumatoid arthritis. Mariette et al., Arthritis Rheum, 50:1270-1276 (2004) reported on a multicenter study with infliximab in treating primary Sjögren's syndrome. The primary endpoint was a decrease of at least 30% in 2 of the 3 VAS (dryness, asthenia and pain). See also Mariette et al., Ann. Rheum. Dis., 62(1): 66-66 (July 2003) reporting the preliminary results of the TRIPSS study that there was an absence of efficiency of infliximab in primary Sjögren's syndrome. Further, Mariette et al., Arthr. and Rheum., 48 Number 9, S260-S260 (September 2003) reported the absence of efficiency of infliximab in primary Sjögren's syndrome resulting from the randomized, double-blind, placebo-controlled TRIPSS study.

In another study, Zandelt et al.; J Rheumatol 31:96-101 (2004) investigated the use of etanercept in primary Sjögren's syndrome and found a marked decrease of fatigue 4/15 (MFI+VAS) and decreased ESR in three out of four endpoints. There was no effect on salivary or lacrimal function+MSG.

In another study, Pillemer et al., Arthritis Rheum 50:2240-2245 (2004) investigated using etanercept in treating Sjögren's syndrome. The results were mild decreased ESR (p=0.004) and no effect on salivary or lacrimal function. Azuma et al., Arthritis Rheum, 46:1585-1594 (2002) disclosed suppression of TNFα-induced MMP9 by cepharantine. Steinfeld et al., Lab Invest 81:143-148 (2001) showed abnormal aquaporin-5 distribution. Towne et al., J Biol Chem, 276:18657-18664 (2001) showed that TNFα inhibits AQP5 expression in mouse lung epithelial cells and that decreased AQP5 mRNA and protein expression in response to TNFα occurs by signaling through the TNFR1 receptor, and decreased AQP5 mRNA and protein expression in response to TNFα require the nuclear translocation of NF-κB. Koski et al., Clin Exp Rheumatol, 19:131-137 (2001) asked which TNFRs are present in the salivary glands.

Although the initial trigger that sets off the autoimmune events leading to Sjögren's remains unknown, circumstantial evidence suggests that a virus is involved. One possible candidate is the Epstein-Barr virus (EBV), which causes infectious mononucleosis, a condition characterized by swollen salivary glands, joint aches, and fatigue. Virtually all adults have been infected with EBV by age 20 years. After the initial infection, this virus normally resides in the salivary glands for life but causes no problems. It has been speculated that this virus (or a closely-related virus) may trigger an autoimmune response in genetically susceptible individuals.

The putative infectious agent damages the salivary gland and attracts the “immune” lymphocytes into the salivary gland. These lymphocytes release specific autoantibodies such as rheumatoid factor (RF), antinuclear antibodies, and antibodies directed against proteins termed Sjögren's-associated antigens A and B (or SS-A and SS-B). Autoantibodies against Ro/SS-A and La/SS-B antigens are present in the tear fluid of some patients with Sjögren's syndrome and their presence in serum or tear fluid is associated with the severity of keratoconjunctivitis sicca. Toker et al. Br J Ophthalmol. 88(3):384-387 (2004). Additionally, antibodies to both centromere protein B (CENP B) and centromere protein C (CENP C) are autoantibodies that occur in Sjögren's syndrome. In a subset representing 15% of Sjögren's syndrome patients studied, these latter anticentromere antibodies recognized exclusively CENP C, and were uniformly associated with antibodies to Ro 52 and La. Pillemer et al. J Rheumatol. 31 (6): 1121-1125 (2004). In addition, Sjögren's syndrome patients have the autoantibody ICA69 (US 2004/0123335).

These antibodies can enter the bloodstream and are measured in the blood tests that are obtained to confirm the diagnosis of Sjögren's syndrome. Additional T cells enter the gland and the damage is perpetuated. Under normal circumstances, a class of cells called “suppressor cells” turn off the inflammatory process. The continued destruction of the gland represents the abnormal balance of excessive action of T-helper cells and deficient action of T-suppressor cells. Hypofunction rather than destruction of these cells is now regarded as the main mechanism of secretory failure in Sjögren's syndrome. Venables, Best Practice & Research. Clin. Rheumatol. 18(3):313-329 (2004).

Better knowledge of the pathogenesis of Sjögren's syndrome and a better understanding of the mechanisms responsible therefor may allow the discovery of new therapeutic strategies. For example, abnormal levels and relative ratios of hormones may play a role in the pathogenesis of Sjögren's syndrome (Taiym et al. Oral Surg, Oral Med, Oral Pathol, Oral Radiol, & Endodontics. 97(5):579-583 (2004)), and women with Sjögren's syndrome are androgen-deficient (Sullivan et al. J Rheumatol. 30 (11):2413-2419 (2003)). Apoptosis is also being studied in Sjögren's syndrome (Manganelli and Fietta, Seminars in Arthritis & Rheumatism 33(1):49-65 (2003)), as well as the role of retroviruses and cytokines and the discovery of aquaporins, to provide new perspectives for the local and systemic management of this disease. Steinfeld and Simonart, Dermatology 207(1):6-9 (2003). Quantification of aquaporin 5 (AQP5) increased only in Sjögren's syndrome patients, suggesting that AQP5 protein leaks into the tears when acinar cells of the lacrimal gland are damaged by lymphocytic infiltration. Ohashi et al. Am J Ophthalmol. 136(2):291-299 (2003). The up-regulation of monokine-induced-by-gamma-interferon, HLA-DR, keratin 6b, -6c, and -16 suggests that in Sjögren's syndrome, interferon-gamma may play an important role in the altered gene expression in the conjunctival epithelium. Kawasaki et al., Exp Eye Res. 77(1):17-26 (2003). Saliva-derived biological mediators may also contribute to increased epithelial cell-proliferative activity observed during inflammation. Ccedilelenligil-Nazliel et al., J Periodontol. 74(2):247-254 (2003).

For further background literature, see, for example, Anaya et al., “Sjögren's syndrome in childhood” J Rheumatol. 22(6):1152-1158 (1995) and Andonopoulos et al., “Sjögren's syndrome in patients with newly diagnosed untreated non-Hodgkin's lymphoma” Rev Rhum Engl Ed. 64(5):287-92 (1997).

As to potential and actual treatments, for example, cevimeline may be useful for dry eye (Ono et al. Am J Ophthalmol. 138(1):6-17 (2004)) as well as diquafosol tetrasodium (Inspire Pharmaceuticals), a formulation of a dinucleotide that functions as an agonist at the P2Y2 receptor, stimulating the release of natural tear components targeting all three mechanisms of action involved in tear secretion—mucin, lipids and fluid, and RESTASIS® (cyclosporine ophthalmic emulsion); and pilocarpine may be useful in salivary enhancement (Fox Caries Res. 38(3):241-246 (2004)). Various immunomodulant treatments based on cyclosporine, corticosteroids, methotrexate, or alpha-interferon have been proposed with different results. Rogers et al., Drugs (New Zealand) 64(2): 123-132 (2004). In a press release, Amarillo Biosciences, Inc. on Jan. 5, 2001 announced completion of a phase III Sjögren's syndrome clinical trial using interferon-alpha, which showed promising results. Immunosuppressive drugs may be useful in some complications of Sjögren's syndrome. Unfortunately, promising results from an open study with infliximab (REMICADE®), a tumor necrosis factor (TNF) antagonist, were not confirmed by a large randomized control study involving more than 100 patients. Xavier et al. Arthritis & Rheum. 50(4):1270-1276 (2004). Further, prominent adverse effects of thalidomide were seen in a study for treating primary Sjögren's syndrome. Pillemer et al., Arthritis & Rheum. 51(3):505-506 (2004). Additionally, a pilot study evaluating the effect of TNF-alpha antiinflammatory treatment with etanercept (ENBREL®), another TNF antagonist, on sicca, systemic, and histological signs in patients with primary Sjögren's syndrome showed that a 12-week or prolonged treatment did not appear to reduce sicca symptoms and signs in Sjögren's syndrome. However, etanercept treatment may be beneficial in a small subgroup of Sjögren's syndrome patients with severe fatigue. Zandbelt et al., J. Rheumatol., 96-101 (2004). Cyclosporine A was found to be efficacious for treating moderate-to-severe dry eye disease. SalI et al., Ophthalmology 107(4): 631-639 (2000); Stevenson et al., Ophthalmology 107(5): 967-974 (2000). The development of topical cyclosporine and other immunomodulating agents has shown promise in the treatment of keratoconjunctivitis sicca in Sjögren's syndrome. Kassan and Moutsopoulos, Archives of Internal Medicine 164(12):1275-1284 (2004). Clinical human gene transfer studies for head and neck cancer treatment of patients to repair damaged salivary glands due to Sjögren's syndrome have been reported. U.S. Newswire dated Oct. 21, 2003. See also WO 2003/68822 published Aug. 21, 2003 regarding use of a polypeptide construct with at least two domains comprising a de-immunized, autoreactive antigen or its fragment that is specifically recognized by the Ig receptors of autoreactive B-cells, for treatment of various autoimmune diseases including Sjögren's syndrome.

### CD20 Antibodies and Treatment Therewith

Lymphocytes are one of many types of white blood cells produced in the bone marrow during the process of hematopoiesis. There are two major populations of lymphocytes: B lymphocytes (B cells) and T lymphocytes (T cells). The lymphocytes of particular interest herein are B cells.

B cells mature within the bone marrow and leave the marrow expressing an antigen-binding antibody on their cell surface. When a naïve B cell first encounters the antigen for which its membrane-bound antibody is specific, the cell begins to divide rapidly and its progeny differentiate into memory B cells and effector cells called “plasma cells”. Memory B cells have a longer life span and continue to express membrane-bound antibody with the same specificity as the original parent cell. Plasma cells do not produce membrane-bound antibody, but instead produce the antibody in a form that can be secreted. Secreted antibodies are the major effector molecules of humoral immunity.

The CD20 antigen (also called human B-lymphocyte-restricted differentiation antigen, Bp35) is a hydrophobic transmembrane protein with a molecular weight of approximately 35 kD located on pre-B and mature B lymphocytes (Valentine et al. J. Biol. Chem. 264(19): 11282-11287 (1989) and Einfeld et al. EMBO J. 7(3):711-717 (1988)). The antigen is also expressed on greater than 90% of B-cell non-Hodgkin's lymphomas (NHL) (Anderson et al. Blood 63(6):1424-1433 (1984)), but is not found on hematopoietic stem cells, pro-B cells, normal plasma cells, or other normal tissues (Tedder et al. J. Immunol. 135(2):973-979 (1985)). CD20 regulates an early step(s) in the activation process for cell-cycle initiation and differentiation (Tedder et al., supra), and possibly functions as a calcium-ion channel (Tedder et al. J. Cell. Biochem. 14D:195 (1990)).

Given the expression of CD20 in B-cell lymphomas, this antigen can serve as a candidate for “targeting” of such lymphomas. In essence, such targeting can be generalized as follows: antibodies specific to the CD20 surface antigen of B cells are administered to a patient. These anti-CD20 antibodies specifically bind to the CD20 antigen of (ostensibly) both normal and malignant B cells; the antibody bound to the CD20 surface antigen may lead to the destruction and depletion of neoplastic B cells. Additionally, chemical agents or radioactive labels having the potential to destroy the tumor can be conjugated to the anti-CD20 antibody such that the agent is specifically “delivered” to the neoplastic B cells. Irrespective of the approach, a primary goal is to destroy the tumor; the specific approach can be determined by the particular anti-CD20 antibody that is utilized, and thus, the available approaches to targeting the CD20 antigen can vary considerably.

The rituximab (RITIUXAN®) antibody is a genetically engineered chimeric murine/human monoclonal antibody directed against the CD20 antigen. Rituximab is the antibody called “C2B8” in U.S. Pat. No. 5,736,137 issued Apr. 7, 1998 (Anderson et al.). Rituximab is indicated for the treatment of patients with relapsed or refractory low-grade or follicular, CD20-positive, B-cell non-Hodgkin's lymphoma. In vitro mechanism-of-action studies have demonstrated that rituximab binds human complement and lyses lymphoid B-cell lines through complement-dependent cytotoxicity (CDC) (Reff et al. Blood 83(2):435-445 (1994)). Additionally, it has significant activity in assays for antibody-dependent cellular cytotoxicity (ADCC). More recently, rituximab has been shown to have anti-proliferative effects in tritiated thymidine-incorporation assays and to induce apoptosis directly, while other anti-CD19 and anti-CD20 antibodies do not (Maloney et al. Blood 88(10):637a (1996)). Synergy between rituximab and chemotherapies and toxins has also been observed experimentally. In particular, rituximab sensitizes drug-resistant human B-cell lymphoma cell lines to the cytotoxic effects of doxorubicin, CDDP, VP-16, diphtheria toxin, and ricin (Demidem et al. Cancer Chemotherapy & Radiopharmaceuticals 12(3):177-186 (1997)). In vivo preclinical studies have shown that rituximab depletes B cells from the peripheral blood, lymph nodes, and bone marrow of cynomolgus monkeys, presumably through complement- and cell-mediated processes (Reff et al. Blood 83(2):435-445 (1994)).

Rituximab was approved in the United States in November 1997 for the treatment of patients with relapsed or refractory low-grade or follicular CD20+B-cell NHL at a dose of 375 mg/m² weekly for four doses. In April 2001, the Food and Drug Administration (FDA) approved additional claims for the treatment of low-grade NHL: retreatment (weekly for four doses) and an additional dosing regimen (weekly for eight doses). There have been more than 300,000 patient exposures to rituximab either as monotherapy or in combination with immunosuppressant or chemotherapeutic drugs. Patients have also been treated with rituximab as maintenance therapy for up to 2 years (Hainsworth et al. J Clin Oncol 21:1746-51 (2003); Hainsworth et al. J Clin Oncol 20:4261-7 (2002)).

Rituximab has also been studied in a variety of non-malignant autoimmune disorders, in which B cells and autoantibodies appear to play a role in disease pathophysiology. Edwards et al., Biochem Soc. Trans. 30:824-828 (2002). Rituximab has been reported to potentially relieve signs and symptoms of, for example, rheumatoid arthritis (RA) (Leandro et al., Ann. Rheum. Dis. 61:883-888 (2002); Edwards et al., Arthritis Rheum., 46 (Suppl. 9): S46 (2002); Stahl et al., Ann. Rheum. Dis., 62 (Suppl. 1): OP004 (2003); Emery et al., Arthritis Rheum. 48(9): S439 (2003)), lupus (Eisenberg, Arthritis. Res. Ther. 5:157-159 (2003); Leandro et al. Arthritis Rheum. 46: 2673-2677 (2002); Gorman et al., Lupus, 13: 312-316 (2004)), immune thrombocytopenic purpura (D'Arena et al., Leuk. Lymphoma 44:561-562 (2003); Stasi et al., Blood, 98: 952-957 (2001); Saleh et al., Semin. Oncol., 27 (Supp 12):99-103 (2000); Zaia et al., Haematolgica, 87: 189-195 (2002); Ratanatharathorn et al., Ann. Int. Med., 133: 275-279 (2000)), pure red cell aplasia (Auner et al., Br. J. Haematol., 116: 725-728 (2002)); autoimmune anemia (Zaja et al., Haematologica 87:189-195 (2002) (erratum appears in Haematologica 87:336 (2002)), cold agglutinin disease (Layios et al., Leukemia, 15: 187-8 (2001); Berentsen et al., Blood, 103: 2925-2928 (2004); Berentsen et al., Br. J. Haematol., 115: 79-83 (2001); Bauduer, Br. J. Haematol., 112: 1083-1090 (2001); Damiani et al., Br. J. Haematol., 114: 229-234 (2001)), type B syndrome of severe insulin resistance (Coll et al., N. Engl. J. Med., 350: 310-311 (2004), mixed cryoglobulinemia (DeVita et al., Arthritis Rheum. 46 Suppl. 9:S206/S469 (2002)), myasthenia gravis (Zaja et al., Neurology, 55: 1062-63 (2000); Wylam et al., J. Pediatr., 143: 674-677 (2003)), Wegener's granulomatosis (Specks et al., Arthritis & Rheumatism 44: 2836-2840 (2001)), refractory pemphigus vulgaris (Dupuy et al., Arch Dermatol., 140:91-96 (2004)), dermatomyositis (Levine, Arthritis Rheum., 46 (Suppl. 9):S1299 (2002)), Sjögren's syndrome (Somer et al., Arthritis & Rheumatism, 49: 394-398 (2003)), active type-II mixed cryoglobulinemia (Zaja et al., Blood, 101: 3827-3834 (2003)), pemphigus vulgaris (Dupay et al., Arch. Dermatol., 140: 91-95 (2004)), autoimmune neuropathy (Pestronk et al., J. Neurol. Neurosurg. Psychiatry 74:485-489 (2003)), paraneoplastic opsoclonus-myoclonus syndrome (Pranzatelli et al. Neurology 60(Suppl. 1) PO5.128:A395 (2003)), and relapsing-remitting multiple sclerosis (RRMS). Cross et al. (abstract) “Preliminary Results from a Phase II Trial of Rituximab in MS” Eighth Annual Meeting of the Americas Committees for Research and Treatment in Multiple Sclerosis, 20-21 (2003).

A Phase II study (WA16291) has been conducted in patients with rheumatoid arthritis (RA), providing 48-week follow-up data on safety and efficacy of Rituximab. Emery et al. Arthritis Rheum 48(9):S439 (2003); Szczepanski et al. Arthritis Rheum 48(9):S121 (2003). A total of 161 patients were evenly randomized to four treatment arms: methotrexate, rituximab alone, rituximab plus methotrexate, and rituximab plus cyclophosphamide (CTX). The treatment regimen of rituximab was one gram administered intravenously on days 1 and 15. Infusions of rituximab in most patients with RA were well tolerated by most patients, with 36% of patients experiencing at least one adverse event during their first infusion (compared with 30% of patients receiving placebo). Overall, the majority of adverse events was considered to be mild to moderate in severity and was well balanced across all treatment groups. There were a total of 19 serious adverse events across the four arms over the 48 weeks, which were slightly more frequent in the rituximab/CTX group. The incidence of infections was well balanced across all groups. The mean rate of serious infection in this RA patient population was 4.66 per 100 patient-years, which is lower than the rate of infections requiring hospital admission in RA patients (9.57 per 100 patient-years) reported in a community-based epidemiologic study. Doran et al., Arthritis Rheum. 46:2287-2293 (2002).

The reported safety profile of rituximab in a small number of patients with neurologic disorders, including autoimmune neuropathy (Pestronk et al., supra), opsoclonus-myoclonus syndrome (Pranzatelli et al., supra), and RRMS (Cross et al., supra), was similar to that reported in oncology or RA. In an ongoing investigator-sponsored trial (IST) of rituximab in combination with interferon-beta (IFN-β) or glatiramer acetate in patients with RRMS (Cross et al., supra), 1 of 10 treated patients was admitted to the hospital for overnight observation after experiencing moderate fever and rigors following the first infusion of rituximab, while the other 9 patients completed the four-infusion regimen without any reported adverse events.

Patents and patent publications concerning CD20 antibodies and CD20-binding molecules include U.S. Pat. Nos. 5,776,456, 5,736,137, 5,843,439, 6,399,061, and 6,682,734, as well as US 2002/0197255, US 2003/0021781, US 2003/0082172, US 2003/0095963, US 2003/0147885 (Anderson et al.); U.S. Pat. No. 6,455,043 and WO 2000/09160 (Grillo-Lopez, A.); WO 2000/27428 (Grillo-Lopez and White); WO 2000/27433 (Grillo-Lopez and Leonard); WO 2000/44788 (Braslawsky et al.); WO 2001/10462 (Rastetter, W.); WO 2001/10461 (Rastetter and White); WO 2001/10460 (White and Grillo-Lopez); US 2001/0018041, US 2003/0180292, WO 2001/34194 (Hanna and Hariharan); US 2002/0006404 and WO 2002/04021 (Hanna and Hariharan); US 2002/0012665, WO 2001/74388 and U.S. Pat. No. 6,896,885B5 (Hanna, N.); US 2002/0058029 (Hanna, N.); US 2003/0103971 (Hariharan and Hanna); US 2005/0123540 (Hanna et al.); US 2002/0009444 and WO 2001/80884 (Grillo-Lopez, A.); WO 2001/97858; US 2005/0112060, and U.S. Pat. No. 6,846,476 (White, C.); US 2002/0128488 and WO 2002/34790 (Reff, M.); WO 2002/060955 (Braslawsky et al.); WO 2002/096948 (Braslawsky et al.); WO 2002/079255 (Reff and Davies); U.S. Pat. No. 6,171,586 and WO 1998/56418 (Lam et al.); WO 1998/58964 (Raju, S.); WO 1999/22764 (Raju, S.); WO 1999/51642, U.S. Pat. No. 6,194,551, U.S. Pat. No. 6,242,195, U.S. Pat. No. 6,528,624 and U.S. Pat. No. 6,538,124 (Idusogie et al.); WO 2000/42072 (Presta, L.); WO 2000/67796 (Curd et al.); WO 2001/03734 (Grillo-Lopez et al.); US 2002/0004587 and WO 2001/77342 (Miller and Presta); US 2002/0197256 (Grewal, I.); US 2003/0157108 (Presta, L.); U.S. Pat. Nos. 6,565,827, 6,090,365, 6,287,537, 6,015,542, 5,843,398, and 5,595,721, (Karninski et al.); U.S. Pat. Nos. 5,500,362, 5,677,180, 5,721,108, 6,120,767, 6,652,852, 6,893,625 (Robinson et al.); U.S. Pat. No. 6,410,391 (Raubitschek et al.); U.S. Pat. No. 6,224,866 and WO00/20864 (Barbera-Guillem, E.); WO 2001/13945 (Barbera-Guillem, E.); WO 2000/67795 (Goldenberg); US 2003/0133930 and WO 2000/74718 (Goldenberg and Hansen); US 2003/0219433 and WO 2003/68821 (Hansen et al.); WO 2004/058298 (Goldenberg and Hansen); WO 2000/76542 (Golay et al.); WO 2001/72333 (Wolin and Rosenblatt); U.S. Pat. No. 6,368,596 (Ghetie et al.); U.S. Pat. No. 6,306,393 and US 2002/0041847 (Goldenberg, D.); US 2003/0026801 (Weiner and Hartmann); WO 2002/102312 (Engleman, E.); US 2003/0068664 (Albitar et al.); WO 2003/002607 (Leung, S.); WO 2003/049694, US 2002/0009427, and US 2003/0185796 (Wolin et al.); WO 2003/061694 (Sing and Siegall); US 2003/0219818 (Bohen et al.); US 2003/0219433 and WO 2003/068821 (Hansen et al.); US 2003/0219818 (Bohen et al.); US 2002/0136719 (Shenoy et al.); WO 2004/032828 (Wahl et al.); and WO 2002/56910 (Hayden-Ledbetter). See also U.S. Pat. No. 5,849,898 and EP 330,191 (Seed et al.); EP332,865A2 (Meyer and Weiss); U.S. Pat. No. 4,861,579 (Meyer et al.); US 2001/0056066 (Bugelski et al.); WO 1995/03770 (Bhat et al.); US 2003/0219433 A1 (Hansen et al.); WO 2004/035607 (Teeling et al.); WO 2004/056312 (Lowman et al.); US 2004/0093621 (Shitara et al.); WO 2004/103404 (Watkins et al.); WO 2005/000901 (Tedder et al.); US 2005/0025764 (Watkins et al.); WO 2005/016969 (Carr et al.); US 2005/0069545 (Carr et al.); WO 2005/014618 (Chang et al.); US 2005/0079174 (Barbera-Guillem and Nelson); US 2005/0106108 (Leung and Hansen); WO2005/044859 and US 2005/0123546 (Umana et al.); and U.S. Pat. No. 6,897,044 (Braslawski et al.).

Publications concerning treatment with rituximab include: Perotta and Abuel, “Response of chronic relapsing ITP of 10 years duration to rituximab” Abstract # 3360 Blood 10(1)(part 1-2): p. 88B (1998); Perotta et al., “Rituxan in the treatment of chronic idiopathic thrombocytopaenic purpura (ITP)”, Blood, 94: 49 (abstract) (1999); Matthews, R., “Medical Heretics” New Scientist (7 April, 2001); Leandro et al., “Clinical outcome in 22 patients with rheumatoid arthritis treated with B lymphocyte depletion” Ann Rheum Dis, supra; Leandro et al., “Lymphocyte depletion in rheumatoid arthritis: early evidence for safety, efficacy and dose response” Arthritis and Rheumatism 44(9): S370 (2001); Leandro et al., “An open study of B lymphocyte depletion in systemic lupus erythematosus”, Arthritis and Rheumatism, 46:2673-2677 (2002), wherein during a 2-week period, each patient received two 500-mg infusions of rituximab, two 750-mg infusions of cyclophosphamide, and high-dose oral corticosteroids, and wherein two of the patients treated relapsed at 7 and 8 months, respectively, and have been retreated, although with different protocols; “Successful long-term treatment of systemic lupus erythematosus with rituximab maintenance therapy” Weide et al., Lupus, 12: 779-782 (2003), wherein a patient was treated with rituximab (375 mg/m²×4, repeated at weekly intervals) and further rituximab applications were delivered every 5-6 months and then maintenance therapy was received with rituximab 375 mg/m² every three months, and a second patient with refractory SLE was treated successfully with rituximab and is receiving maintenance therapy every three months, with both patients responding well to rituximab therapy; Edwards and Cambridge, “Sustained improvement in rheumatoid arthritis following a protocol designed to deplete B lymphocytes” Rheumatology 40:205-211 (2001); Cambridge et al., “B lymphocyte depletion in patients with rheumatoid arthritis: serial studies of immunological parameters” Arthritis Rheum., 46 (Suppl. 9): S1350 (2002); Edwards et al., “B-lymphocyte depletion therapy in rheumatoid arthritis and other autoimmune disorders” Biochem Soc. Trans., supra; Edwards et al., “Efficacy and safety of rituximab, a B-cell targeted chimeric monoclonal antibody: A randomized, placebo controlled trial in patients with rheumatoid arthritis. Arthritis and Rheumatism 46(9): S197 (2002); Edwards et al., “Efficacy of B-cell-targeted therapy with rituximab in patients with rheumatoid arthritis” N Engl. J. Med. 350:2572-82 (2004); Pavelka et al., Ann. Rheum. Dis. 63: (S1):289-90 (2004); Emery et al., Arthritis Rheum. 50 (S9):S659 (2004); Levine and Pestronk, “IgM antibody-related polyneuropathies: B-cell depletion chemotherapy using rituximab” Neurology 52: 1701-1704 (1999); DeVita et al., “Efficacy of selective B cell blockade in the treatment of rheumatoid arthritis” Arthritis & Rheum 46:2029-2033 (2002); Hidashida et al. “Treatment of DMARD-refractory rheumatoid arthritis with rituximab.” Presented at the Annual Scientific Meeting of the American College of Rheumatology; October 24-29; New Orleans, La. 2002; Tuscano, J. “Successful treatment of infliximab-refractory rheumatoid arthritis with rituximab” Presented at the Annual Scientific Meeting of the American College of Rheumatology; October 24-29; New Orleans, La. 2002; “Pathogenic roles of B cells in human autoimmunity; insights from the clinic” Martin and Chan, Immunity 20:517-527 (2004); Silverman and Weisman, “Rituximab therapy and autoimmune disorders, prospects for anti-B cell therapy”, Arthritis and Rheumatism, 48: 1484-1492 (2003); Kazkaz and Isenberg, “Anti B cell therapy (rituximab) in the treatment of autoimmune diseases”, Current opinion in pharmacology, 4: 398-402 (2004); Virgolini and Vanda, “Rituximab in autoimmune diseases”, Biomedicine & pharmacotherapy, 58: 299-309(2004); Klemmer et al., “Treatment of antibody mediated autoimmune disorders with a AntiCD20 monoclonal antibody Rituximab”, Arthritis And Rheumatism, 48: (9) 9,S (SEP), page: S624-S624 (2003); Kneitz et al., “Effective B cell depletion with rituximab in the treatment of autoimmune diseases”, Immunobiology, 206: 519-527 (2002); Arzoo et al., “Treatment of refractory antibody mediated autoimmune disorders with an anti-CD20 monoclonal antibody (rituximab) “Annals of the Rheumatic Diseases, 61 (10), p922-4 (2002) Comment in Ann Rheum Dis. 61: 863-866 (2002); “Future strategies in immunotherapy” by Lake and Dionne, in Burger's Medicinal Chemistry and Drug Discovery (2003 by John Wiley & Sons, Inc.) Article Online Posting Date: Jan. 15, 2003 (Chapter 2” Antibody-Directed Immunotherapy”); Liang and Tedder, Wiley Encyclopedia of Molecular Medicine, Section: CD20 as an Immunotherapy Target, article online posting date: 15 Jan. 2002 entitled “CD20”; Appendix 4A entitled “Monoclonal Antibodies to Human Cell Surface Antigens” by Stockinger et al., eds: Coligan et al., in Current Protocols in Immunology (2003 John Wiley & Sons, Inc) Online Posting Date: May, 2003; Print Publication Date: February, 2003; Penichet and Morrison, “CD Antibodies/molecules: Definition; Antibody Engineering” in Wiley Encyclopedia of Molecular Medicine Section: Chimeric, Humanized and Human Antibodies; posted online 15 Jan. 2002; Specks et al. “Response of Wegener's granulomatosis to anti-CD20 chimeric monoclonal antibody therapy” Arthritis & Rheumatism 44:2836-2840 (2001); online abstract submission and invitation Koegh et al., “Rituximab for Remission Induction in Severe ANCA-Associated Vasculitis: Report of a Prospective Open-Label Pilot Trial in 10 Patients”, American College of Rheumatology, Session Number: 28-100, Session Title: Vasculitis, Session Type: ACR Concurrent Session, Primary Category: 28 Vasculitis, Session Oct. 18, 2004 (<www.abstractsonline.com/viewer/SearchResults.asp>); Eriksson, “Short-term outcome and safety in 5 patients with ANCA-positive vasculitis treated with rituximab”, Kidney and Blood Pressure Research, 26: 294 (2003); Jayne et al., “B-cell depletion with rituximab for refractory vasculitis” Kidney and Blood Pressure Research, 26: 294 (2003); Jayne, poster 88 (11^{th} International Vasculitis and ANCA workshop), 2003 American Society of Nephrology; Stone and Specks, “Rituximab therapy for the induction of remission and tolerance in ANCA-associated vasculitis”, in the Clinical Trial Research Summary of the 2002-2003 Immune Tolerance Network, <www.immunetolerance.org/research/autoimmune/trials/stone.html>. See also Leandro et al., “B cell repopulation occurs mainly from naïve B cells in patient with rheumatoid arthritis and systemic lupus erythematosus” Arthritis Rheum., 48 (Suppl 9): S1160 (2003).

Further, see Looney “B cells as a therapeutic target in autoimmune diseases other than rheumatoid arthritis” Rheumatology, 44 Suppl 2: ii13-ii17 (2005); Chambers and Isenberg, “Anti-B cell therapy (rituximab) in the treatment of autoimmune diseases” Lupus 14(3): 210-214 (2005); Edelbauer et al., “Rituximab in childhood systemic lupus erythematosus refractory to conventional immunosuppression Case report” Pediatr. Nephrol. 20(6): 811-813 (2005); D'Cruz and Hughes, “The treatment of lupus nephritis” BMJ 330(7488): 377-378 (2005); Looney, “B cell-targeted therapy in diseases other than rheumatoid arthritis” J. Rheumatol. Suppl. 73: 25-28; discussion 29-30 (2005); Sfikakis et al., “Remission of proliferative lupus nephritis following B cell depletion therapy is preceded by down-regulation of the T cell costimulatory molecule CD40 ligand: an open-label trial” Arthritis Rheum. 52(2): 501-513 (2005); Silverman, “Anti-CD20 therapy in systemic lupus erythematosus: a step closer to the clinic” Arthritis Rheum. 52(2): 371-7 (2005), Erratum in: Arthritis Rheum. 52(4): 1342 (2005); Ahn et al., “Long-term remission from life-threatening hypercoagulable state associated with lupus anticoagulant (LA) following rituximab therapy” Am. J. Hematol. 78(2): 127-129 (2005); Tahir et al., “Humanized anti-CD20 monoclonal antibody in the treatment of severe resistant systemic lupus erythematosus in a patient with antibodies against rituximab” Rheumatology, 44(4): 561-562 (2005), Epub 2005 Jan. 11; Looney et al., “Treatment of SLE with anti-CD20 monoclonal antibody” Curr. Dir. Autoimmun. 8: 193-205 (2005); Cragg et al., “The biology of CD20 and its potential as a target for mAb therapy” Curr. Dir. Autoimmun. 8: 140-174 (2005); Gottenberg et al., “Tolerance and short term efficacy of rituximab in 43 patients with systemic autoimmune diseases” Ann. Rheum. Dis. 64(6): 913-920 (2005) Epub 2004 Nov. 18; Tokunaga et al., “Down-regulation of CD40 and CD80 on B cells in patients with life-threatening systemic lupus erythematosus after successful treatment with rituximab” Rheumatology 44(2): 176-182 (2005), Epub 2004 Oct. 19. Several cases of serum sickness-like syndrome have been observed in rituximab investigator-sponsored trials involving Sjögren's syndrome that, without being limited to any one theory, may be related to the chimeric nature of the antibody and/or apoptosis of B-cells leading to cytokine release. Further, in Sjögren's patients elevated levels of BAFF might lead to anti-apoptotic tendency, increased BAFF levels correlate with hypergammaglobulinemia, and there are increased B-cell cytokine levels. See also US 2005/0053602 published Mar. 10, 2005 regarding treatment of ocular disorders, e.g. Sjögren's eye complication, with a CD20 antagonist, as well as WO 2003/014294; US 2005/0070689 published Mar. 31, 2005; US 2003/0095967 published May 22, 2003; US 2005/0095243 published May 5, 2005; and WO 2005/005462 published Jan. 20, 2005.

Presently, no therapies are available to cure the underlying causes of Sjögren's syndrome, and no disease-modifying anti-rheumatic drugs (DMARDs) are approved for treating Sjögren's syndrome. Therapies are thus directed at improving symptoms, preventing complications (e.g. dental caries, oral candida, or corneal damage), and preventing disease progression. There is a slightly increased risk of developing lymphoma (tumor of the lymph nodes), so careful attention is paid to persistent swelling of these structures. People afflicted with Sjögren's syndrome need a cost-efficient and safe treatment that will help ameliorate their condition.

### SUMMARY OF THE INVENTION

The present invention involves administration of a CD20 antibody that provides a safe and active treatment regimen in subjects with Sjögren's syndrome, including selection of an efficacious dosing regimen.

Accordingly, the invention is as claimed. In a first aspect, the present invention concerns a method of treating Sjögren's syndrome in a patient comprising administering an effective amount of a CD20 antibody and an anti-malarial agent to the patient to provide at least about 30% improvement over baseline in two or more of dryness, fatigue, and joint pain on a Visual Analogue Scale (VAS).

In a further aspect, the invention provides an article of manufacture comprising: a container comprising a CD20 antibody; a container comprising an anti-malarial agent; and a package insert with instructions for treating Sjögren's syndrome in a patient, wherein the instructions indicate that amounts of the CD20 antibody and the anti-malarial agent are administered to the patient that are effective to provide at least about 30% improvement over baseline in two or more of dryness, fatigue, and joint pain on a Visual Analogue Scale.

In preferred embodiments of the above inventive aspects, a third medicament is administered in an effective amount to the patient, wherein the CD20 antibody is a first medicament and the anti-malarial agent is a second medicament. More preferably, such third medicament is a chemotherapeutic agent, an immunosuppressive agent, a disease-modifying anti-rheumatic drug (DMARD), a cytotoxic agent, an integrin antagonist, a nonsteroidal antiinflammatory drug (NSAID), a cytokine antagonist, a secretory agonist for dry mouth or dry eye, or a hormone. In another aspect, the patient has relapsed before being administered the CD20 antibody. In a further aspect, the patient has not relapsed before being administered the CD20 antibody. In a still further preferred aspect, the syndrome is secondary Sjögren's syndrome.

In still further aspects, the present invention relates to a method of treating Sjögren's syndrome in a subject comprising administering an effective amount of a CD20 antibody to the subject to provide an initial antibody exposure followed by a second antibody exposure, wherein the second exposure is not provided until from about 16 to 54 weeks from the initial exposure.

In one preferred embodiment of this lattermost aspect, the present invention relates to a method of treating Sjögren's syndrome in a subject comprising administering an effective amount of a CD20 antibody to the subject to provide an initial antibody exposure of about 0.5 to 4 grams followed by a second antibody exposure of about 0.5 to 4 grams, wherein the second exposure is not provided until from about 16 to 54 weeks from the initial exposure and each of the antibody exposures is provided to the subject as about 1 to 4 doses of antibody, more preferably as a single dose or as two or three separate doses of antibody.

In another preferred embodiment of this lattermost aspect, a second medicament is administered with the initial exposure and/or later exposures, wherein the CD20 antibody is a first medicament. In a preferred embodiment, the second medicament is a chemotherapeutic agent, an immunosuppressive agent, a disease-modifying anti-rheumatic drug (DMARD), a cytotoxic agent, an integrin antagonist, a nonsteroidal antiinflammatory drug (NSAID), a cytokine antagonist, a secretory agonist for dry mouth or dry eye, or a hormone. In a more preferred embodiment, the second medicament is an anti-malarial agent alone or with a steroid or is a steroid. In a still preferred embodiment, a steroid is administered with the first exposure, but not with the second exposure, or is administered in lower amounts than are used with the initial exposure.

In still another preferred embodiment of this lattermost aspect, the subject has never been previously treated with a CD20 antibody, and/or no other medicament than the CD20 antibody is administered to the subject to treat the Sjögren's syndrome.

In yet another preferred embodiment of this lattermost aspect, the subject has an elevated level of anti-nuclear antibodies (ANA), anti-rheumatoid factor (RF) antibodies, antibodies directed against Sjögren's-associated antigen A or B (SS-A or SS-B), antibodies directed against centromere protein B (CENP B) or centromere protein C (CENP C), an autoantibody to ICA69, or a combination of two or more of such antibodies. More preferably, the antibodies directed against SS-A and SS-B are anti-Ro/SS-A antibodies, anti-La/SS-A antibodies, anti-La/SS-B antibodies, or anti-Ro/SS-B antibodies.

Additionally, in further aspects, the invention provides an article of manufacture comprising:

(a) a container comprising a CD20 antibody; and

(b) a package insert with instructions for treating Sjögren's syndrome in a subject, wherein the instructions indicate that an amount of the antibody is administered to the subject that is effective to provide an initial antibody exposure followed by a second antibody exposure, wherein the second exposure is not provided until from about 16 to 54 weeks from the initial exposure.

Preferably, such package insert is provided with instructions for treating Sjögren's syndrome in a subject, wherein the instructions indicate that an amount of the antibody is administered to the subject that is effective to provide an initial antibody exposure of about 0.5 to 4 grams followed by a second antibody exposure of about 0.5 to 4 grams, wherein the second exposure is not provided until from about 16 to 54 weeks from the initial exposure and each of the antibody exposures is provided to the subject as about one to four doses, preferably as a single dose or as two or three separate doses of antibody.

The treatments herein preferably reduce, minimize, or eliminate the need for co-, pre-, or post-administration of excessive amounts of second or third medicaments such as immunosuppressive agents or chemotherapeutic agents that are ordinarily standard treatment for such subjects, to avoid as much as possible the side effects of such standard treatment, as well as reduce costs and increase convenience to the subject, such as time convenience.

### BRIEF DESCRIPTION OF THE DRAWINGS

FIG. 1A is a sequence alignment comparing the amino acid sequences of the light chain variable domain (V_{L}) of each of murine 2H7 (SEQ ID NO:1), humanized 2H7.v16 variant (SEQ ID NO:2), and the human kappa light chain subgroup I (SEQ ID NO:3). The CDRs of V_{L} of 2H7 and hu2H7.v16 are as follows: CDR1 (SEQ ID NO:4), CDR2 (SEQ ID NO:5), and CDR3 (SEQ ID NO:6).

FIG. 1B is a sequence alignment comparing the amino acid sequences of the heavy chain variable domain (V_{H}) of each of murine 2H7 (SEQ ID NO:7), humanized 2H7.v16 variant (SEQ ID NO:8), and the human consensus sequence of the heavy chain subgroup III (SEQ ID NO:9). The CDRs of V_{H} of 2H7 and hu2H7.v16 are as follows: CDR1 (SEQ ID NO:10), CDR2 (SEQ ID NO:11), and CDR3 (SEQ ID NO:12).

In FIG. 1A and FIG. 1B, the CDR1, CDR2 and CDR3 in each chain are enclosed within brackets, flanked by the framework regions, FR1—FR4, as indicated. 2H7 refers to the murine 2H7 antibody. The asterisks in between two rows of sequences indicate the positions that are different between the two sequences. Residue numbering is according to Kabat et al. Sequences of Immunological Interest, 5th Ed. Public Health Service, National Institutes of Health, Bethesda, Md. (1991), with insertions shown as a, b, c, d, and e.

FIG. 2 shows the amino acid sequence of the mature 2H7.v16 L chain (SEQ ID NO:13)

FIG. 3 shows the amino acid sequence of the mature 2H7.v16H chain (SEQ ID NO:14).

FIG. 4 shows the amino acid sequence of the mature 2H7.v31H chain (SEQ ID NO:15). The L chain of 2H7.v31 is the same as for 2H7.v16.

FIG. 5 shows an alignment of the mature 2H7.v16 and 2H7.v511 light chains (SEQ ID NOS: 13 and 16, respectively), with Kabat variable-domain residue numbering and Eu constant-domain residue numbering.

FIG. 6 shows an alignment of the mature 2H7.v16 and 2H7.v511 heavy chains (SEQ ID NOS:14 and 17, respectively), with Kabat variable-domain residue numbering and Eu constant-domain residue numbering.

### DETAILED DESCRIPTION OF THE PREFERRED EMBODIMENTS

I. Definitions

“Sjögren's syndrome” as used herein is an autoimmune disease or disorder in which immune cells attack the glands that produce tears and saliva. The hallmark symptoms of the disorder are dry mouth and dry eyes. In addition, Sjögren's syndrome may cause skin, nose, and vaginal dryness, and may affect other organs of the body including the kidneys, blood vessels, lungs, liver, pancreas, and brain. Sjögren's syndrome can exist as a primary disorder (“primary Sjögren's syndrome”) or as a secondary disorder (“secondary Sjögren's syndrome”) that is associated with and/or secondary to other autoimmune disorders including rheumatic disorders such as rheumatoid arthritis, systemic lupus, polymyositis, scleroderma, and autoimmune hepatitis, lymphomas such as non-Hodgkin's lymphoma, and endocrine disorders such as thyroiditis. The term “Sjögren's syndrome” as used herein applies to Sjögren's syndrome no matter what the stage, including both primary and secondary Sjögren's syndrome, and no matter what symptoms are evident, provided the diagnosis is made. Diagnoses for the syndrome include those set forth below. It also includes subjects with moderate-severe sicca symptoms without systemic manifestations as well as subjects with systemic symptoms.

A “B cell” is a lymphocyte that matures within the bone marrow, and includes a naïve B cell, memory B cell, or effector B cell (plasma cells). The B cell herein may be a normal or non-malignant B cell.

A “B-cell surface marker” or “B-cell surface antigen” herein is an antigen expressed on the surface of a B cell that can be targeted with an antagonist that binds thereto. Exemplary B-cell surface markers include the CD10, CD19, CD20, CD21, CD22, CD23, CD24, CD37, CD40, CD53, CD72, CD73, CD74, CDw75, CDw76, CD77, CDw78, CD79a, CD79b, CD80, CD81, CD82, CD83, CDw84, CD85 and CD86 leukocyte surface markers (for descriptions, see The Leukocyte Antigen Facts Book, 2^{nd} Edition. 1997, ed. Barclay et al. Academic Press, Harcourt Brace & Co., New York). Other B-cell surface markers include RP105, FcRH2, B-cell CR2, CCR6, P2×5, HLA-DOB, CXCR5, FCER2, BR3, Btig, NAG14, SLGC16270, FcRH1, IRTA2, ATWD578, FcRH3, IRTA1, FcRH6, BCMA, and 239287. The B-cell surface marker of particular interest is preferentially expressed on B cells compared to other non-B-cell tissues of a mammal and may be expressed on both precursor B cells and mature B cells. The preferred B-cell surface markers herein are CD20 and CD22.

The “CD20” antigen, or “CD20,” is an about 35-kDa, non-glycosylated phosphoprotein found on the surface of greater than 90% of B cells from peripheral blood or lymphoid organs. CD20 is present on both normal B cells as well as malignant B cells, but is not expressed on stem cells. Other names for CD20 in the literature include “B-lymphocyte-restricted antigen” and “Bp35”. The CD20 antigen is described in Clark et al. Proc. Natl. Acad. Sci. (USA) 82:1766 (1985), for example.

The “CD22” antigen, or “CD22,” also known as BL-CAM or Lyb8, is a type 1 integral membrane glycoprotein with molecular weight of about 130 (reduced) to 140 kD (unreduced). It is expressed in both the cytoplasm and cell membrane of B-lymphocytes. CD22 antigen appears early in B-cell lymphocyte differentiation at approximately the same stage as the CD19 antigen. Unlike other B-cell markers, CD22 membrane expression is limited to the late differentiation stages comprised between mature B cells (CD22+) and plasma cells (CD22−). The CD22 antigen is described, for example, in Wilson et al. J. Exp. Med. 173:137 (1991) and Wilson et al. J. Immunol. 150:5013 (1993).

An “antagonist” is a molecule that, upon binding to CD20 on B cells, destroys or depletes B cells in a mammal and/or interferes with one or more B cell functions, e.g. by reducing or preventing a humoral response elicited by the B cell. The antagonist preferably is able to deplete B cells (i.e. reduce circulating B cell levels) in a mammal treated therewith. Such depletion may be achieved via various mechanisms such antibody-dependent cell-mediated cytotoxicity (ADCC) and/or complement dependent cytotoxicity (CDC), inhibition of B cell proliferation and/or induction of B cell death (e.g. via apoptosis). Antagonists included within the scope of the present invention include antibodies, synthetic or native-sequence peptides, immunoadhesins, and small-molecule antagonists that bind to CD20, optionally conjugated with or fused to a cytotoxic agent. The preferred antagonist comprises an antibody.

An “antibody antagonist” herein is an antibody that, upon binding to a B-cell surface marker on B cells, destroys or depletes B cells in a mammal and/or interferes with one or more B-cell functions, e.g., by reducing or preventing a humoral response elicited by the B cell. The antibody antagonist preferably is able to deplete B cells (i.e., reduce circulating B-cell levels) in a mammal treated therewith. Such depletion may be achieved via various mechanisms such antibody-dependent cell-mediated cytotoxicity (ADCC) and/or complement-dependent cytotoxicity (CDC), inhibition of B-cell proliferation and/or induction of B-cell death (e.g., via apoptosis).

The term “antibody” herein is used in the broadest sense and specifically covers monoclonal antibodies, polyclonal antibodies, multispecific antibodies (e.g. bispecific antibodies) formed from at least two intact antibodies, and antibody fragments so long as they exhibit the desired biological activity.

“Antibody fragments” comprise a portion of an intact antibody, preferably comprising the antigen binding region thereof. Examples of antibody fragments include Fab, Fab′, F(ab′)₂, and Fv fragments; diabodies; linear antibodies; single-chain antibody molecules; and multispecific antibodies formed from antibody fragments.

For the purposes herein, an “intact antibody” is one comprising heavy and light variable domains as well as an Fc region.

An “antibody that binds to a B-cell surface marker” is a molecule that, upon binding to a B-cell surface marker, destroys or depletes B cells in a mammal and/or interferes with one or more B-cell functions, e.g. by reducing or preventing a humoral response elicited by the B cell. The antibody preferably is able to deplete B cells (i.e. reduce circulating B-cell levels) in a mammal treated therewith. Such depletion may be achieved via various mechanisms such antibody-dependent cell-mediated cytotoxicity (ADCC) and/or complement-dependent cytotoxicity (CDC), inhibition of B-cell proliferation and/or induction of B-cell death (e.g. via apoptosis). Preferably, the B-cell surface marker is CD20, so that the antibody that binds to a B-cell surface marker is an antibody that binds to CD20, or a “CD20 antibody.”

Examples of CD20 antibodies include: “C2B8,” which is now called “rituximab” (“RITUXAN®”) (U.S. Pat. No. 5,736,137); the yttrium-[90]-labeled 2B8 murine antibody designated “Y2B8” or “Ibritumomab Tiuxetan” (ZEVALIN®) commercially available from IDEC Pharmaceuticals, Inc. (U.S. Pat. No. 5,736,137; 2B8 deposited with ATCC under accession no. HB11388 on Jun. 22, 1993); murine IgG2a “B1,” also called “Tositumomab,” optionally labeled with ¹³¹I to generate the “131I-B1” or “iodine I131 tositumomab” antibody (BEXXAR™) commercially available from Corixa (see, also, U.S. Pat. No. 5,595,721); murine monoclonal antibody “1F5” (Press et al. Blood 69(2):584-591 (1987) and variants thereof including “framework patched” or humanized IF5 (WO 2003/002607, Leung, S.; ATCC deposit HB-96450); murine 2H7 and chimeric 2H7 antibody (U.S. Pat. No. 5,677,180); humanized 2H7; HUMAX-CD20™ fully human, high-affinity antibody targeted at the CD20 molecule in the cell membrane of B-cells (Genmab, Denmark; see, for example, Glennie and van de Winkel, Drug Discovery Today 8: 503-510 (2003) and Cragg et al., Blood 101: 1045-1052 (2003)); the human monoclonal antibodies set forth in WO04/035607 (Teeling et al.); AME-133™ antibodies (Applied Molecular Evolution); A20 antibody or variants thereof such as chimeric or humanized A20 antibody (cA20, hA20, respectively) (US 2003/0219433, Immunomedics); and monoclonal antibodies L27, G28-2,93-1B3, B-C1 or NU-B2 available from the International Leukocyte Typing Workshop (Valentine et al., In: Leukocyte Typing III (McMichael, Ed., p. 440, Oxford University Press (1987)). The preferred CD20 antibodies herein are chimeric, humanized, or human CD20 antibodies, more preferably rituximab, humanized 2H7, chimeric or humanized A20 antibody (Immunomedics), and HUMAX-CD20™ human CD20 antibody (Genmab).

The terms “rituximab” or “RITUXAN®” herein refer to the genetically engineered chimeric murine/human monoclonal antibody directed against the CD20 antigen and designated “C2B8” in U.S. Pat. No. 5,736,137, including fragments thereof which retain the ability to bind CD20.

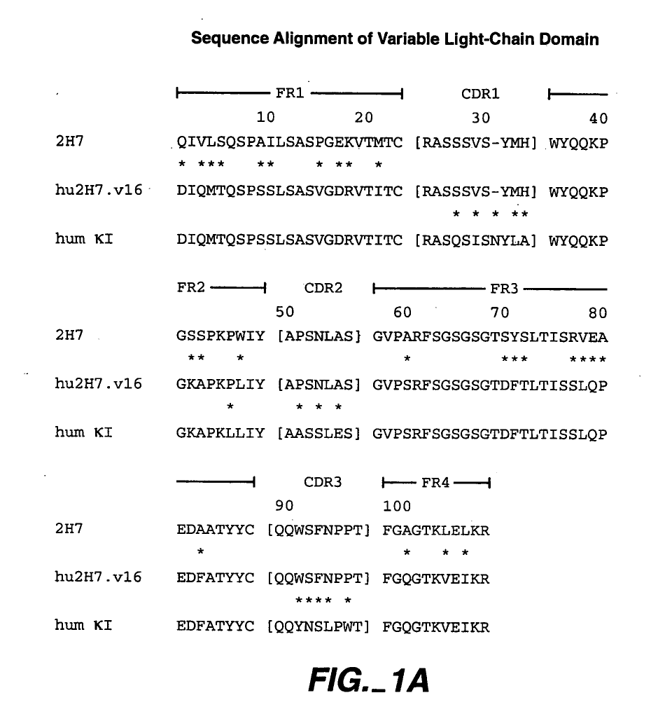

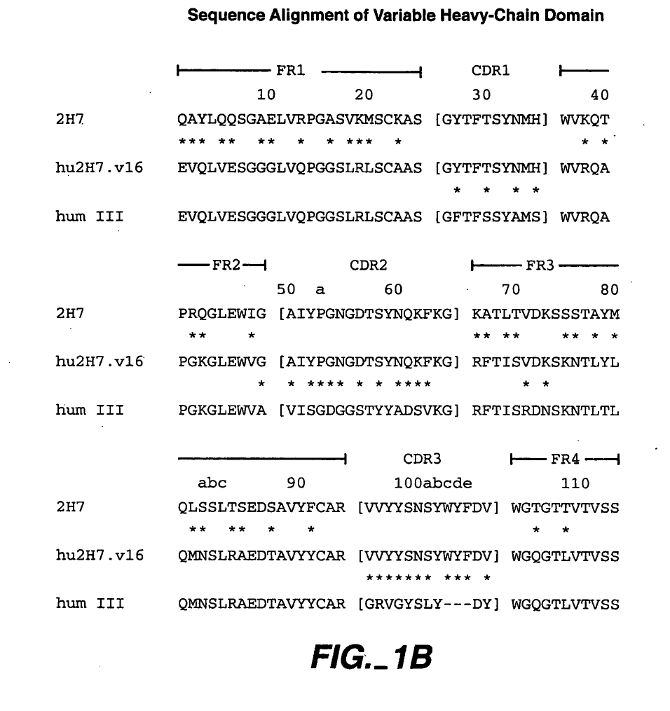

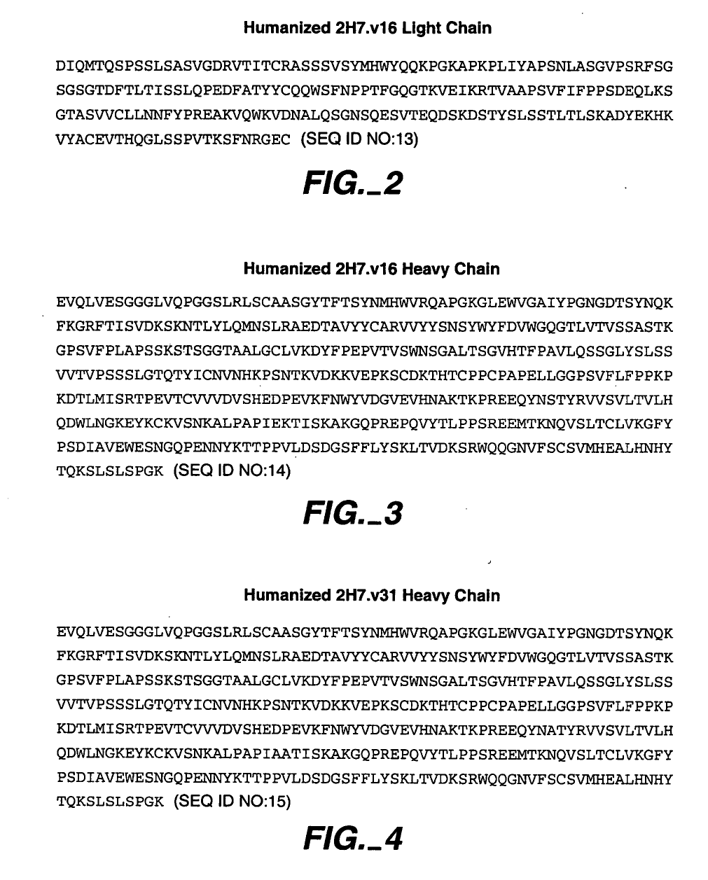

Purely for the purposes herein and unless indicated otherwise, “humanized 2H7” refers to a humanized CD20 antibody, or an antigen-binding fragment thereof, wherein the antibody is effective to deplete primate B cells in vivo, the antibody comprising in the H chain variable region (V_{H}) thereof at least a CDR H3 sequence of SEQ ID NO:12 (FIG. 1B) from an anti-human CD20 antibody and substantially the human consensus framework (FR) residues of the human heavy-chain subgroup III (V_{H}III). In a preferred embodiment, this antibody further comprises the H chain CDR H1 sequence of SEQ ID NO:10 and CDR H2 sequence of SEQ ID NO:11, and more preferably further comprises the L chain CDR L1 sequence of SEQ ID NO:4, CDR L2 sequence of SEQ ID NO:5, CDR L3 sequence of SEQ ID NO:6 and substantially the human consensus framework (FR) residues of the human light chain subgroup I (VI), wherein the V_{H} region may be joined to a human IgG chain constant region, wherein the region may be, for example, IgG1 or IgG3. In a preferred embodiment, such antibody comprises the V_{H} sequence of SEQ ID NO:8 (v16, as shown in FIG. 1B), optionally also comprising the V_{L} sequence of SEQ ID NO:2 (v16, as shown in FIG. 1A), which may have the amino acid substitutions of D56A and N100A in the H chain and S92A in the L chain (v96). Preferably the antibody is an intact antibody comprising the light and heavy chain amino acid sequences of SEQ ID NOS:13 and 14, respectively, as shown in FIGS. 2 and 3. Another preferred embodiment is where the antibody is 2H7.v31 comprising the light and heavy chain amino acid sequences of SEQ ID NOS:13 and 15, respectively, as shown in FIGS. 2 and 4. The antibody herein may further comprise at least one amino acid substitution in the Fc region that improves ADCC and/or CDC activity, such as one wherein the amino acid substitutions are S298A/E333A/K334A, more preferably 2H7.v31 having the heavy chain amino acid sequence of SEQ ID NO:15 (as shown in FIG. 4). Any of these antibodies may further comprise at least one amino acid substitution in the Fc region that decreases CDC activity, for example, comprising at least the substitution K322A. See U.S. Pat. No. 6,528,624B1 (Idusogie et al.).

A preferred humanized 2H7 is an intact antibody or antibody fragment comprising the variable light chain sequence:

| | |
|---|---|
| DIQMTQSPSSLSASVGDRVTITCRASSSVSYMHWYQQKPGKAPKPLIYAPSNLASGVPSRFSGSGSGTD | (SEQ ID NO: 2) |
| FTLTISSLQPEDFATYYCQQWSFNPPTFGQGTKVEIKR; | |

and the variable heavy chain sequence:

| | |
|---|---|
| EVQLVESGGGLVQPGGSLRLSCAASGYTFTSYNMHWVRQAPGKGLEWVGAIYPGNGDTSYNQKFKG | (SEQ ID NO: 8) |
| RFTISVDKSKNTLYLQMNSLRAEDTAVYYCARVVYYSNSYWYFDVWGQGTLVTVSS. | |

Where the humanized 2H7 antibody is an intact antibody, preferably it comprises the light chain amino acid sequence:

| | |
|---|---|
| DIQMTQSPSSLSASVGDRVTITCRASSSVSYMHWYQQKPGKAPKPLIYAPSNLASGVPSRFSGSGSGTD | (SEQ ID NO: 13) |
| FTLTISSLQPEDFATYYCQQWSFNPPTFGQGTKVEIKRTVAAPSVFIFPPSDEQLKSGTASVVCLLNNFYP | |
| REAKVQWKVDNALQSGNSQESVTEQDSKDSTYSLSSTLTLSKADYEKHKVYACEVTHQGLSSPVTKS | |
| FNRGEC; | |

and the heavy chain amino acid sequence:

| | |
|---|---|
| EVQLVESGGGLVQPGGSLRLSCAASGYTFTSYNMHWVRQAPGKGLEWVGAIYPGNGDTSYNQKFKG | (SEQ ID NO: 14) |
| RFTISVDKSKNTLYLQMNSLRAEDTAVYYCARVVYYSNSYWYFDVWGQGTLVTVSSASTKGPSVFPL | |
| APSSKSTSGGTAALGCLVKDYFPEPVTVSWNSGALTSGVHTFPAVLQSSGLYSLSSVVTVPSSSLGTQT | |
| YICNVNHKPSNTKVDKKVEPKSCDKTHTCPPCPAPELLGGPSVFLFPPKPKDTLMISRTPEVTCVVVDV | |
| SHEDPEVKFNWYVDGVEVHNAKTKPREEQYNSTYRVVSVLTVLHQDWLNGKEYKCKVSNKALPAPI | |
| EKTISKAKGQPREPQVYTLPPSREEMTKNQVSLTCLVKGFYPSDIAVEWESNGQPENNYKTTPPVLDSD | |
| GSFFLYSKLTVDKSRWQQGNVFSCSVMHEALHNHYTQKSLSLSPGK | |

or the heavy chain amino acid sequence:

| | |
|---|---|
| EVQLVESGGGLVQPGGSLRLSCAASGYTFTSYNMHWVRQAPGKGLEWVGAIYPGNGDTSYNQKFKG | (SEQ ID NO: 15) |
| RFTISVDKSKNTLYLQMNSLRAEDTAVYYCARVVYYSNSYWYFDVWGQGTLVTVSSASTKGPSVFPL | |
| APSSKSTSGGTAALGCLVKDYFPEPVTVSWNSGALTSGVHTFPAVLQSSGLYSLSSVVTVPSSSLGTQT | |
| YICNVNHKPSNTKVDKKVEPKSCDKTHTCPPCPAPELLGGPSVFLFPPKPKDTLMISRTPEVTCVVVDV | |
| SHEDPEVKFNWYVDGVEVHNAKTKPREEQYNSTYRVVSVLTVLHQDWLNGKEYKCKVSNKALPAPI | |
| EKTISKAKGQPREPQVYTLPPSREEMTKNQVSLTCLVKGFYPSDIAVEWESNGQPENNYKTTPPVLDSD | |
| GSFFLYSKLTVDKSRWQQGNVFSCSVMHEALHNHYTQKSLSLSPGK | |

In the preferred embodiment of the invention, the V region of variants based on 2H7 version 16 will have the amino acid sequences of v16 except at the positions of amino acid substitutions that are indicated in the table below. Unless otherwise indicated, the 2H7 variants will have the same L chain as that of v16.

| 2H7 | Heavy chain | Light chain | |
|---|---|---|---|
| Version | (V_{H}) changes | (V_{L}) changes | Fc changes |
| 31 | — | — | S298A, E333A, K334A |
| 96 | D56A, N100A | S92A | |
| 114 | D56A, N10 | M32L, S92A | S298A, E333A, K334A |
| 115 | D56A, N100A | M32L, S92A | S298A, E333A, K334A, |
| | | | E356D, M358L |

“Antibody-dependent cell-mediated cytotoxicity” and “ADCC” refer to a cell-mediated reaction in which nonspecific cytotoxic cells that express Fc receptors (FcRs) (e.g. Natural Killer (NK) cells, neutrophils, and macrophages) recognize bound antibody on a target cell and subsequently cause lysis of the target cell. The primary cells for mediating ADCC, NK cells, express FcγRIII only, whereas monocytes express FcγRI, FcγRII and FcγRIII. FcR expression on hematopoietic cells in summarized is Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or U.S. Pat. No. 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. PNAS (USA) 95:652-656 (1998).

“Human effector cells” are leukocytes that express one or more FcRs and perform effector functions. Preferably, the cells express at least FcγRIII and carry out ADCC effector function. Examples of human leukocytes that mediate ADCC include peripheral blood mononuclear cells (PBMC), natural killer (NK) cells, monocytes, cytotoxic T cells and neutrophils; with PBMCs and NK cells being preferred.

The terms “Fc receptor” or “FcR” are used to describe a receptor that binds to the Fc region of an antibody. The preferred FcR is a native-sequence human FcR. Moreover, a preferred FcR is one that binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (see Daëron, Annu. Rev. Immunol. 15:203-234 (1997)). FcRs are reviewed in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976) and Kim et al., J. Immunol. 24:249 (1994)).

“Complement dependent cytotoxicity” or “CDC” refer to the ability of a molecule to lyse a target in the presence of complement. The complement activation pathway is initiated by the binding of the first component of the complement system (C1q) to a molecule (e.g. an antibody) complexed with a cognate antigen. To assess complement activation, a CDC assay, e.g. as described in Gazzano-Santoro et al., J. Immunol. Methods 202:163 (1996), may be performed.

“Growth-inhibitory” antibodies are those that prevent or reduce proliferation of a cell expressing an antigen to which the antibody binds. For example, the antibody may prevent or reduce proliferation of B cells in vitro and/or in vivo.

Antibodies that “induce apoptosis” are those that induce programmed cell death, e.g. of a B cell, as determined by standard apoptosis assays, such as binding of annexin V, fragmentation of DNA, cell shrinkage, dilation of endoplasmic reticulum, cell fragmentation, and/or formation of membrane vesicles (called apoptotic bodies).

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain. Particular amino acid residues are believed to form an interface between the light chain and heavy chain variable domains.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light chain and the heavy chain variable domains. The more highly conserved portions of variable domains are called the framework regions (FRs). The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC).

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-binding sites and is still capable of cross-linking antigen.

“Fv” is the minimum antibody fragment that contains a complete antigen-recognition and antigen-binding site. This region consists of a dimer of one heavy chain and one light chain variable domain in tight, non-covalent association. It is in this configuration that the three hypervariable regions of each variable domain interact to define an antigen-binding site on the surface of the V_{H}-V_{L} dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site.

The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear at least one free thiol group. F(ab′)₂ antibody fragments originally were produced as pairs of Fab′ fragments that have hinge cysteines between them. Other chemical couplings of antibody fragments are also known.

The “light chains” of antibodies (immunoglobulins) from any vertebrate species can be assigned to one of two clearly distinct types, called kappa (κ) and lambda (λ), based on the amino acid sequences of their constant domains.

Depending on the amino acid sequence of the constant domain of their heavy chains, antibodies can be assigned to different classes. There are five major classes of intact antibodies: IgA, IgD, IgE, IgG, and IgM, and several of these may be further divided into subclasses (isotypes), e.g., IgG1, IgG2, IgG3, IgG4, IgA, and IgA2. The heavy chain constant domains that correspond to the different classes of antibodies are called α, δ, ε, γ, and μ, respectively. The subunit structures and three-dimensional configurations of different classes of immunoglobulins are well known.

“Single-chain Fv” or “scFv” antibody fragments comprise the V_{H} and V_{L} domains of antibody, wherein these domains are present in a single polypeptide chain. Preferably, the Fv polypeptide further comprises a polypeptide linker between the V_{H} and V_{L} domains that enables the scFv to form the desired structure for antigen binding. For a review of scFv see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (V_{H}) connected to a light-chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

The term “monoclonal antibody” as used herein refers to an antibody obtained from a population of substantially homogeneous antibodies, i.e., the individual antibodies comprising the population are identical and/or bind the same epitope, except for possible variants that may arise during production of the monoclonal antibody, such variants generally being present in minor amounts. In contrast to polyclonal antibody preparations that typically include different antibodies directed against different determinants (epitopes), each monoclonal antibody is directed against a single determinant on the antigen. In addition to their specificity, the monoclonal antibodies are advantageous in that they are uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature, 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature, 352:624-628 (1991) and Marks et al., J. Mol. Biol., 222:581-597 (1991), for example.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81:6851-6855 (1984)). Chimeric antibodies of interest herein include “primatized” antibodies comprising variable domain antigen-binding sequences derived from a non-human primate (e.g. Old World Monkey, such as baboon, rhesus or cynomolgus monkey) and human constant region sequences (U.S. Pat. No. 5,693,780).

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence, except for FR substitution(s) as noted above. The humanized antibody optionally also will comprise at least a portion of an immunoglobulin constant region, typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

The term “hypervariable region” when used herein refers to the amino acid residues of an antibody that are responsible for antigen binding. The hypervariable region comprises amino acid residues from a “complementarity determining region” or “CDR” (e.g. residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (e.g. residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)). “Framework” or “FR” residues are those variable domain residues other than the hypervariable region residues as herein defined.

A “naked antibody” is an antibody (as herein defined) that is not conjugated to a heterologous molecule, such as a cytotoxic moiety or radiolabel.

An “isolated” antibody is one that has been identified and separated and/or recovered from a component of its natural environment. Contaminant components of its natural environment are materials that would interfere with diagnostic or therapeutic uses for the antibody, and may include enzymes, hormones, and other proteinaceous or non-proteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

A “subject” herein is a human subject, including a patient, eligible for treatment for Sjögren's syndrome who is experiencing or has experienced one or more signs, symptoms, or other indicators of Sjögren's syndrome, has been diagnosed with Sjögren's syndrome, whether, for example, newly diagnosed or previously diagnosed and now experiencing a recurrence or relapse, or is at risk for developing Sjögren's syndrome. The subject may have been previously treated with CD20 antibody or not so treated. A subject eligible for treatment of Sjögren's syndrome may optionally be identified as one who has been screened, as in the blood, for elevated levels of infiltrating CD20 cells or is screened using an assay to detect auto-antibodies, wherein autoantibody production is assessed qualitatively, and preferably quantitatively. Exemplary such auto-antibodies associated with Sjögren's syndrome include anti-nuclear antibodies (ANA), anti-rheumatoid factor (RF) antibodies, antibodies directed against proteins termed Sjögren's-associated antigens A or B (or SS-A or SS-B), such as, for example, anti-Ro/SS-A antibodies, anti-La/SS-A antibodies, anti-La/SS-B antibodies, and anti-Ro/SS-B antibodies, antibodies directed against centromere protein B (CENP B) or centromere protein C (CENP C), an autoantibody to ICA69, or a combination of two or more of such antibodies.

A “patient” herein is a human subject eligible for treatment for Sjögren's syndrome who is experiencing or has experienced one or more signs, symptoms, or other indicators of Sjögren's syndrome, whether, for example, newly diagnosed or previously diagnosed and now experiencing a recurrence or relapse. The patient may have been previously treated with CD20 antibody or not so treated. A patient eligible for treatment of Sjögren's syndrome may optionally be identified as one who has been screened, as in the blood, for elevated levels of infiltrating CD20 cells or is screened using an assay to detect auto-antibodies, such as those noted above, wherein autoantibody production is assessed qualitatively, and preferably quantitatively.

Several diagnostic tests are commonly used in people suspected of having Sjögren's syndrome. Such tests include clinical examination of the eyes and mouth Two well-accepted tests can be performed by an ophthalmologist to test for dry eyes: 1. Schirmer's test, which involves numbing the eye from being irritated before placing a strip of paper (referred to as a Schirmer's strip) in the eye. This strip measures the amount of wetting that occurs over a five-minute period. Less than 5 mm of wetting is a strong indicator of dry eyes. This test is not 100% accurate and should be performed again if the diagnosis remains an issue. 2. Rose-Bengal dye test, which stains for damaged/inflamed areas of the cornea.

Dry mouth can be checked by measuring salivary gland flow rates to determine whether there is decreased saliva production. In some patients, the infiltration of lymphocytes into the parotid or submandibular glands causes pain and swelling. To determine the extent of salivary gland destruction associated with oral dryness, a biopsy may be taken from the inner surface of the lower lip to establish a firm diagnosis to show how many (if any) salivary glands remain and the type of inflammatory infiltrate present. A positive result reveals characteristic inflammatory features consistent with the diagnosis of Sjögren's syndrome. It is likely that the mouth and eye dryness results both from destruction of the salivary glands and from interruption of nerve signals that control secretion. In the early stages of Sjögren's syndrome, patients experience maximum dryness between meals and during the night due to a diminished “basal” secretion, but are still able to eat dry food without difficulty. As the “dryness” syndrome progresses, more fluid is required to eat and swallow. The diminished salivary flow also predisposes to periodontal disease and oral yeast infections such as Candida. Severe sensitivity to spicy foods and alcohol is a common complaint; in the same way, mouthwashes and dental products containing essential oils, such as eugenol, may be intolerable.

Although Sjögren's syndrome characteristically affects the eyes and the mouth, other parts of the body may also be affected. Joint and muscle pain are frequently present. In some cases, this is due to rheumatoid arthritis (RA), systemic lupus erythematosus (SLE) or SLE-like diseases. These latter diagnoses are confirmed, for example, by blood tests and x-rays of the joint. However, in some cases, the muscle and joint pain is due to Sjögren's syndrome.

Fatigue is another common symptom. It is important to rule out hypothyroidism (which may develop in up to 20% of Sjögren's syndrome patients), anemia (due to decreased production of blood cells as well as blood loss from taking medicines such as aspirin, ibuprofen, or naproxen for the joint pains), and poor sleeping patterns (especially due to frequent trips to the bathroom at night because of large oral fluid intake during the day). Decrease in memory and concentration sometimes occurs and may be due to the release of inflammatory substances by the immune system. They can also occur due to disrupted sleep patterns. Skin rashes, lung inflammation, swollen lymph nodes, and other symptoms also occur.

In addition, quantification of aquaporins such as aquaporin 5 (AQP5) may be useful in diagnosis of this syndrome.

“Treatment” of a subject herein refers to both therapeutic treatment and prophylactic or preventative measures. Those in need of treatment include those already with Sjögren's syndrome as well as those in which the Sjögren's syndrome is to be prevented. Hence, the subject may have been diagnosed as having the Sjögren's syndrome or may be predisposed or susceptible to the Sjögren's syndrome. Treatment of a subject includes treatment of a patient.

“Treatment” of a patient herein refers to therapeutic treatment. Those patients in need of treatment are those diagnosed with Sjögren's syndrome.

A “symptom” of Sjögren's syndrome is any morbid phenomenon or departure from the normal in structure, function, or sensation, experienced by the subject or patient and indicative of disease.

The expression “effective amount” refers to an amount of the antibody or antagonist that is effective for treating Sjögren's syndrome.

“Antibody exposure” refers to contact with or exposure to the antibody herein in one or more doses administered over a period of time of about 1 day to about 5 weeks. The doses may be given at one time or at a fixed or at irregular time intervals over this period of exposure, such as, for example, one dose weekly for four weeks or two doses separated by a time interval of about 13-17 days. Initial and later antibody exposures are separated in time from each other as described in detail herein.

An exposure not being administered or provided until a certain time “from the initial exposure” or from any prior exposure means that the time for the second or later exposure is measured from the time any of the doses from the prior exposure were administered, if more than one dose was administered in that exposure. For example, when two doses are administered in an initial exposure, the second exposure is not given until at least about 16-54 weeks as measured from the time the first or the second dose was administered within that prior exposure. Similarly, when three doses are administered, the second exposure may be measured from the time of the first, second, or third dose within the prior exposure. Preferably, “from the initial exposure” or from any prior disclosure is measured from the time of the first dose.

The term “immunosuppressive agent” as used herein for adjunct therapy refers to substances that act to suppress or mask the immune system of the mammal being treated herein. This would include substances that suppress cytokine production, down-regulate or suppress self-antigen expression, or mask the MHC antigens. Examples of such agents include 2-amino-6-aryl-5-substituted pyrimidines (see U.S. Pat. No. 4,665,077); nonsteroidal antiinflammatory drugs (NSAIDs); ganciclovir, tacrolimus, glucocorticoids such as cortisol or aldosterone, anti-inflammatory agents such as a cyclooxygenase inhibitor, a 5-lipoxygenase inhibitor, or a leukotriene receptor antagonist; purine antagonists such as azathioprine or mycophenolate mofetil (MMF); alkylating agents such as cyclophosphamide; bromocryptine; danazol; dapsone; glutaraldehyde (which masks the MHC antigens, as described in U.S. Pat. No. 4,120,649); anti-idiotypic antibodies for MHC antigens and MHC fragments; cyclosporin A; steroids such as corticosteroids or glucocorticosteroids or glucocorticoid analogs, e.g., prednisone, methylprednisolone, and dexamethasone; dihydrofolate reductase inhibitors such as methotrexate (oral or subcutaneous); antimalarial agents such as chloroquine and hydroxychloroquine; sulfasalazine; leflunomide; cytokine or cytokine receptor antibodies including anti-interferon-alpha, -beta, or -gamma antibodies, anti-tumor necrosis factor(TNF)-alpha antibodies (infliximab or adalimumab), anti-TNF-alpha immunoadhesin (etanercept), anti-TNF-beta antibodies, anti-interleukin-2 (IL-2) antibodies and anti-IL-2 receptor antibodies, and anti-interleukin-6 (IL-6) receptor antibodies and antagonists; anti-LFA-1 antibodies, including anti-CD11a and anti-CD18 antibodies; anti-L3T4 antibodies; heterologous anti-lymphocyte globulin; pan-T antibodies, preferably anti-CD3 or anti-CD4/CD4a antibodies; soluble peptide containing a LFA-3 binding domain (WO 90/08187 published Jul. 26, 1990); streptokinase; transforming growth factor-beta (TGF-beta); streptodornase; RNA or DNA from the host; FK506; RS-61443; chlorambucil; deoxyspergualin; rapamycin; T-cell receptor (Cohen et al., U.S. Pat. No. 5,114,721); T-cell receptor fragments (Offner et al., Science, 251: 430-432 (1991); WO 90/11294; Ianeway, Nature, 341: 482 (1989); and WO 91/01133); BAFF antagonists such as BAFF antibodies and BR3 antibodies; anti-CD40 receptor or anti-CD40 ligand (CD154); and T-cell receptor antibodies (EP 340,109) such as T10B9. Some preferred immunosuppressive agents herein include cyclophosphamide, chlorambucil, azathioprine, or methotrexate.

The term “cytotoxic agent” as used herein refers to a substance that inhibits or prevents the function of cells and/or causes destruction of cells. The term is intended to include radioactive isotopes (e.g. At²¹¹, I¹³¹, I¹²⁵, Y⁹⁰, Re¹⁸⁶, Re¹⁸⁸, Sm¹⁵³, Bi²¹², P³² and radioactive isotopes of Lu), chemotherapeutic agents, and toxins such as small-molecule toxins or enzymatically active toxins of bacterial, fungal, plant or animal origin, or fragments thereof.

A “chemotherapeutic agent” is a chemical compound useful in the treatment of cancer. Examples of chemotherapeutic agents include alkylating agents such as thiotepa and cyclosphosphamide (CYTOXAN®); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (see, e.g., Agnew, Chem Intl. Ed. Engl., 33: 183-186 (1994)); dynemicin, including dynemicin A; an esperamicin; as well as neocarzinostatin chromophore and related chromoprotein enediyne antiobiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HCl liposome injection (DOXIL®) and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoids, e.g., paclitaxel (TAXOL®), albumin-engineered nanoparticle formulation of paclitaxel (ABRAXANE™), and doxetaxel (TAXOTERE®); chloranbucil; 6-thioguanine; mercaptopurine; methotrexate; platinum analogs such as cisplatin and carboplatin; vinblastine (VELBAN®); platinum; etoposide (VP-16); ifosfamide; mitoxantrone; vincristine (ONCOVIN®); oxaliplatin; leucovovin; vinorelbine (NAVELBINE®); novantrone; edatrexate; daunomycin; aminopterin; ibandronate; topoisomerase inhibitor RFS 2000; difluorometlhylornithine (DMFO); retinoids such as retinoic acid; pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU and leucovovin.

Also included in this definition are anti-hormonal agents that act to regulate, reduce, block, or inhibit the effects of hormones that can promote the growth of cancer, and are often in the form of systemic, or whole-body treatment. They may be hormones themselves. Examples include anti-estrogens and selective estrogen receptor modulators (SERMs), including, for example, tamoxifen (including NOLVADEX® tamoxifen), raloxifene (EVISTA®), droloxifene, 4-hydroxytamoxifen, trioxifene, keoxifene, LY117018, onapristone, and toremifene (FARESTON®); anti-progesterones; estrogen receptor down-regulators (ERDs); estrogen receptor antagonists such as fulvestrant (FASLODEX®); agents that function to suppress or shut down the ovaries, for example, leutinizing hormone-releasing hormone (LHRH) agonists such as leuprolide acetate (LUPRON® and ELIGARD®), goserelin acetate, buserelin acetate and tripterelin; anti-androgens such as flutamide, nilutamide and bicalutamide; and aromatase inhibitors that inhibit the enzyme aromatase, which regulates estrogen production in the adrenal glands, such as, for example, 4(5)-imidazoles, aminoglutethimide, megestrol acetate (MEGASE®), exemestane (AROMASIN®), formestanie, fadrozole, vorozole (RIVISOR®), letrozole (FEMARA®), and anastrozole (ARIMIDEX®). In addition, such definition of chemotherapeutic agents includes bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); as well as troxacitabine (a 1,3-dioxolane nucleoside cytosine analog); antisense oligonucleotides, particularly those that inhibit expression of genes in signaling pathways implicated in abherant cell proliferation, such as, for example, PKC-alpha, Raf, H-Ras, and epidermal growth factor receptor (EGF-R); vaccines such as THERATOPE® vaccine and gene therapy vaccines, for example, ALLOVECTIN® vaccine, LEUVECTIN® vaccine, and VAXID® vaccine; topoisomerase 1 inhibitor (e.g., LURTOTECAN®); rmRH (e.g., ABARELIX®); lapatinib ditosylate (an ErbB-2 and EGFR dual tyrosine kinase small-molecule inhibitor also known as GW572016); COX-2 inhibitors such as celecoxib (CELEBREX®; 4-(5-(4-methylphenyl)-3-(trifluoromethyl)-1H-pyrazol-1-yl)benzenesulfonamide; and pharmaceutically acceptable salts, acids or derivatives of any of the above.

The term “cytokine” is a generic term for proteins released by one cell population that act on another cell as intercellular mediators. Examples of such cytokines are lymphokines, monokines; interleukins (ILs) such as IL-1, IL-1a, IL-2, IL-3, IL-4, IL-5, IL-6, IL-7, IL-8, IL-9, IL-11, IL-12, IL-15, including PROLEUKIN® rIL-2; a tumor necrosis factor such as TNF-αor TNF-β; and other polypeptide factors including LIF and kit ligand (KL). As used herein, the term cytokine includes proteins from natural sources or from recombinant cell culture and biologically active equivalents of the native-sequence cytokines, including synthetically produced small-molecule entities and pharmaceutically acceptable derivatives and salts thereof.

The term “hormone” refers to polypeptide hormones, which are generally secreted by glandular organs with ducts. Included among the hormones are, for example, growth hormone such as human growth hormone, N-methionyl human growth hormone, and bovine growth hormone; parathyroid hormone; thyroxine; insulin; proinsulin; relaxin; estradiol; hormone-replacement therapy; androgens such as calusterone, dromostanolone propionate, epitiostanol, mepitiostane, or testolactone; prorelaxin; glycoprotein hormones such as follicle stimulating hormone (FSH), thyroid stimulating hormone (TSH), and luteinizing hormone (LH); prolactin, placental lactogen, mouse gonadotropin-associated peptide, gonadotropin-releasing hormone; inhibin; activin; mullerian-inhibiting substance; and thrombopoietin. As used herein, the term hormone includes proteins from natural sources or from recombinant cell culture and biologically active equivalents of the native-sequence hormone, including synthetically produced small-molecule entities and pharmaceutically acceptable derivatives and salts thereof.

The term “growth factor” refers to proteins that promote growth, and include, for example, hepatic growth factor; fibroblast growth factor; vascular endothelial growth factor; nerve growth factors such as NGF-β; platelet-derived growth factor; transforming growth factors (TGFs) such as TGF-α and TGF-β; insulin-like growth factor-I and -II; erythropoietin (EPO); osteoinductive factors; interferons such as interferon-α, -β, and -γ; and colony stimulating factors (CSFs) such as macrophage-CSF (M-CSF); granulocyte-macrophage-CSF (GM-CSF); and granulocyte-CSF (G-CSF). As used herein, the term growth factor includes proteins from natural sources or from recombinant cell culture and biologically active equivalents of the native-sequence growth factor, including synthetically produced small-molecule entities and pharmaceutically acceptable derivatives and salts thereof.

The term “integrin” refers to a receptor protein that allows cells both to bind to and to respond to the extracellular matrix and is involved in a variety of cellular functions such as wound healing, cell differentiation, homing of tumor cells and apoptosis. They are part of a large family of cell adhesion receptors that are involved in cell-extracellular matrix and cell-cell interactions. Functional integrins consist of two transmembrane glycoprotein subunits, called alpha and beta, that are non-covalently bound. The alpha subunits all share some homology to each other, as do the beta subunits. The receptors always contain one alpha chain and one beta chain. Examples include Alpha6beta1, Alpha3beta1, Alpha7beta1, LFA-1 etc. As used herein, the term integrin includes proteins from natural sources or from recombinant cell culture and biologically active equivalents of the native-sequence integrin, including synthetically produced small-molecule entities and pharmaceutically acceptable derivatives and salts thereof.

For the purposes herein, “tumor necrosis factor alpha (TNF-alpha)” refers to a human TNF-alpha molecule comprising the amino acid sequence as described in Pennica et al., Nature, 312:721 (1984) or Aggarwal et al., JBC, 260:2345 (1985).

A “TNF-alpha inhibitor” herein is an agent that inhibits, to some extent, a biological function of TNF-alpha, generally through binding to TNF-alpha and neutralizing its activity. Examples of TNF inhibitors specifically contemplated herein are etanercept (ENBREL®), infliximab (REMICADE®), and adalimumab (HUMIRA™).

Examples of “disease-modifying anti-rheumatic drugs” or “DMARDs” include hydroxycloroquine, sulfasalazine, methotrexate, leflunomide, etanercept, infliximab (plus oral and subcutaneous methrotrexate), azathioprine, D-penicillamine, gold salts (oral), gold salts (intramuscular), minocycline, cyclosporine including cyclosporine A and topical cyclosporine, staphylococcal protein A immunoadsorption, including salts and derivatives thereof, etc.

Examples of “nonsteroidal anti-inflammatory drugs” or “NSAIDs” are aspirin, acetylsalicylic acid, ibuprofen, naproxen, indomethacin, sulindac, tolmetin, including salts and derivatives thereof, etc. Preferably, they are aspirin, naproxen, ibuprofen, indomethacin, or tolmetin.

Examples of “integrin antagonists or antibodies” herein include an LFA-1 antibody, such as efalizumab (RAPTIVA®) commercially available from Genentech, or an alpha 4 integrin antibody such as natalizumab (ANTEGREN®) available from Biogen, or diazacyclic phenylalanine derivatives (WO 2003/89410), phenylalanine derivatives (WO 2003/70709, WO 2002/28830, WO 2002/16329 and WO 2003/53926), phenylpropionic acid derivatives (WO 2003/10135), enamine derivatives (WO 2001/79173), propanoic acid derivatives (WO 2000/37444), alkanoic acid derivatives (WO 2000/32575), substituted phenyl derivatives (U.S. Pat. Nos. 6,677,339 and 6,348,463), aromatic amine derivatives (U.S. Pat. No. 6,369,229), ADAM disintegrin domain polypeptides (US2002/0042368), antibodies to alphavbeta3 integrin (EP 633945), aza-bridged bicyclic amino acid derivatives (WO 2002/02556), etc.

“Secretory agonist for dry mouth or dry eye” is a medicament for treating dry mouth or dry eye, such as, for example, pilocarpine and pilocarpine hydrochloride, cevimeline (EVOXAC®), bromhexine, RESTASIS® (cyclosporine ophthalmic emulsion), diquafosol, purinergic receptor agonists, muscarinic agonists, parasympathomimetic agents, cysteamine eye drops (Kaiser-Kupfer et al., Arch Ophthalmol., 108(5): 689-693 (1990)), REFRESH ENDURA® lubricant eye drops, and their pharmaceutical salts and derivatives.

“Corticosteroid” refers to any one of several synthetic or naturally occurring substances with the general chemical structure of steroids that mimic or augment the effects of the naturally occurring corticosteroids. Examples of synthetic corticosteroids include prednisone, prednisolone (including methylprednisolone), dexamethasone or dexamethasone triamcinolone, hydrocortisone, and betamethasone. The preferred corticosteroids herein are prednisone, methylprednisolone, hydrocortisone, or dexamethasone.

An “antimalarial agent” is an agent that treats malaria (including prevention of malaria), and is useful, for example, to treat the systemic complications of Sjögren's syndrome, such as arthritis, fatigue, and skin rashes. Such agent includes, for example, hydrochloroquine, chloroquine, LARIUM™, mefloquine, mefloquine hydrochloride, MEPHAQUINE™, primaquine—ATABRINE™, mepacrine, quinacrine, quinacrine hydrochloride, and quinine. Preferably, it is hydrochloroquine or chloroquine, most preferably hydroxychloroquine (such as the brand name PLAQUENIL®).

The terms “BAFF,” “BAFF polypeptide,” “TALL-1” or “TALL-1 polypeptide,” and “BLyS” when used herein encompass “native-sequence BAFF polypeptides” and “BAFF variants”. “BAFF” is a designation given to those polypeptides that have any one of the amino acid sequences shown below:

| Human BAFF sequence (SEQ ID NO: 16): | |
|---|---|
| 1 | MDDSTEREQSRLTSCLKKREEMKLKECVSILPRKESPSVRSSKDGKLLAATLLLALLSCC |
| 61 | LTVVSFYQVAALQGDLASLRAELQGHHAEKLPAGAGAPKAGLEEAPAVTAGLKIFEPPAP |
| 121 | GEGNSSQNSRNKRAVQGPEETVTQDCLQLIADSETPTIQKGSYTFVPWLLSFKRGSALEE |
| 181 | KENKILVKETGYFFIYGQVLYTDKTYAMGHLIQRKKVHVFGDELSLVTLFRCIQNMPETL |
| 241 | PNNSCYSAGIAKLEEGDELQLAIPRENAQISLDGDVTFFGALKLL |

| Mouse BAFF sequence (SEQ ID NO: 17): | |
|---|---|
| 1 | MDESAKTLPPPCLCFCSEKGEDMKVGYDPITPQKEEGAWFGICRDGRLLAATLLLALLSS |
| 61 | SFTAMSLYQLAALQADLMNLRMELQSYRGSATPAAAGAPELTAGVKLLTPAAPRPHNSSR |
| 121 | GHRNRRAFQGPEETEQDVDLSAPPAPCLPGCRHSQHDDNGMNLRNIIQDCLQLIADSDTP |
| 181 | TIRKGTYTFVPWLLSFKRGNALEEKENKIVVRQTGYFFIYSQVLYTDPIFAMGHVIQRKK |
| 241 | VHVFGDELSLVTLFRCIQNMPKTLPNNSCYSAGIARLEEGDEIQLAIPRENAQISRNGDD |
| 301 | TFFGALKLL |

and homologs and fragments and variants thereof, which have the biological activity of the native BAFF. A biological activity of BAFF can be selected from the group consisting of promoting B cell survival, promoting B cell maturation and binding to BR3. Variants of BAFF will preferably have at least 80% or any successive integer up to 100% including, more preferably, at least 90%, and even more preferably, at least 95% amino acid sequence identity with a native sequence of a BAFF polypeptide.

A “native-sequence” BAFF polypeptide comprises a polypeptide having the same amino acid sequence as the corresponding BAFF polypeptide derived from nature. For example, BAFF exists in a soluble form following cleavage from the cell surface by furin-type proteases. Such native-sequence BAFF polypeptides can be isolated from nature or can be produced by recombinant and/or synthetic means.

The term “native-sequence BAFF polypeptide” or “native BAFF” specifically encompasses naturally occurring truncated or secreted forms (e.g., an extracellular domain sequence), naturally occurring variant forms (e.g., alternatively spliced forms), and naturally occurring allelic variants of the polypeptide. The term “BAFF” includes those polypeptides described in Shu et al., J. Leukocyte Biol., 65:680 (1999); GenBank Accession No. AF136293; WO 1998/18921 published May 7, 1998; EP 869,180 published Oct. 7, 1998; WO 1998/27114 published Jun. 25, 1998; WO 1999/12964 published Mar. 18, 1999; WO 1999/33980 published Jul. 8, 1999; Moore et al., Science, 285:260-263 (1999); Schneider et al., J. Exp. Med., 189:1747-1756 (1999) and Mukhopadhyay et al., J. Biol. Chem., 274:15978-15981 (1999).

The term “BAFF antagonist” as used herein is used in the broadest sense, and includes any molecule that (1) binds a native-sequence BAFF polypeptide or binds a native-sequence of BR3 to partially or fully block BR3 interaction with BAFF polypeptide, and (2) partially or fully blocks, inhibits, or neutralizes native-sequence BAFF activity. In one preferred embodiment the BAFF receptor to be blocked is the BR3 receptor. Native BAFF activity promotes, among other things, B-cell survival and/or B-cell maturation. In one embodiment, the inhibition, blockage or neutralization of BAFF activity results in a reduction in the number of B cells. A BAFF antagonist according to this invention will partially or fully block, inhibit, or neutralize one or more biological activities of a BAFF polypeptide, in vitro and/or in vivo. In one embodiment, a biologically active BAFF potentiates any one or a combination of the following events in vitro and/or in vivo: an increased survival of B cells, an increased level of IgG and/or IgM, an increased numbers of plasma cells, and processing of NF-κb2/100 to p52 NF-κb in splenic B cells (e.g., Batten et al., J. Exp. Med. 192:1453-1465 (2000); Moore et al., Science 285:260-263 (1999); Kayagaki et al. Immunity 17:515-524 (2002)).

As mentioned above, a BAFF antagonist can function in a direct or indirect manner to partially or fully block, inhibit or neutralize BAFF signaling, in vitro or in vivo. For instance, the BAFF antagonist can directly bind BAFF. For example, BAFF antibodies that bind within a region of human BAFF comprising residues 162-275 and/or a neighboring residue of a residue selected from the group consisting of 162, 163, 206, 211, 231, 233, 264 and 265 of human BAFF such that the antibody sterically hinders BAFF binding to BR3 are contemplated, where such residue numbers refer to SEQ ID NO:16. In another example, a direct binder is a polypeptide comprising any portion of a BAFF receptor that binds BAFF such as an extracellular domain of a BAFF receptor, or fragments and variants thereof that bind native BAFF. In another example, BAFF antagonists include the polypeptides having a sequence of a polypeptide comprising the sequence of Formula I:

(SEQ ID NO: 18)

X₁-C-X₃-D-X₅-L-X₇-X₈-X₉-X₁₀-X₁₁-X₁₂-C-X₁₄-X₁₅-X₁₆-
X₁₇ (Formula I)

wherein X₁, X₃, X₅, X₇, X₈, X₉, X₁₀, X₁₁, X₁₂, X₁₄, X₁₅ and X₁₇ are any amino acid except cysteine; and
wherein X₁₆ is an amino acid selected from the group consisting of L, F, I and V; and
wherein the polypeptide does not comprise a cysteine within seven amino acid residues N-terminal to the most N-terminal cysteine C and C-terminal to the most C-terminal cysteine C of Formula I.

In one embodiment, a polypeptide comprising the sequence of Formula I has the two Cs joined by disulfide bonding; X₅LX₇X₈ forming the conformation of a type I beta turn structure with the center of the turn between L and X₇; and has a positive value for the dihedral angle phi of X₈. In one embodiment, X₁₀ is selected from the group consisting of W, F, V, L, I, Y, M and a non-polar amino amino acid. In another embodiment, X₁₀ is W. In another embodiment, X₃ is an amino acid selected from the group consisting of M, V, L, I, Y, F, W and a non-polar amino acid. In another embodiment, X₅ is selected from the group consisting of V, L, P, S, I, A and R. In another embodiment, X₇ is selected from the group consisting of V, T, I and L. In another embodiment, X₈ is selected from the group consisting of R, K, G, N, H and a D-amino acid. In another embodiment, X₉ is selected from the group consisting of H, K, A, R and Q. In another embodiment, X₁₁ is I or V. In another embodiment, X₁₂ is selected from the group consisting of P, A, D, E and S. In another embodiment, X₁₆ is L. In one specific embodiment, the sequence of Formula I is a sequence selected from the group consisting of ECFDLLVRAWVPCSVLK (SEQ ID NO:19), ECFDLLVRHWVPCGLLR (SEQ ID NO:20), ECFDLLVRRWVPCEMLG (SEQ ID NO:21), ECFDLLVRSWVPCHMLR (SEQ ID NO:22), ECFDLLVRHWVACGLLR (SEQ ID NO:23), and QCFDRLNAWVPCSVLK (SEQ ID NO:24). In a preferred embodiment, the BAFF antagonist comprises any one of the amino acid sequences selected from the group consisting of SEQ ID NO: 19, 20, 21, 22, and 23.

In still another example, BAFF antagonists include the polypeptides having a sequence of a polypeptide comprising the sequence of Formula II:

X₁-C-X₃-X₅-L-V-X₈-X₉-W-V-P-C-X₁₄-X₁₅-L-X₁₇
(Formula II) (SEQ ID NO: 25)

wherein X₁, X₃, X₅, X₈, X₉, X₁₄, X₁₅ and X₁₇ are any amino acid, except cysteine; and

wherein the polypeptide does not comprise a cysteine within seven amino acid residues N-terminal to the most N-terminal cysteine C and C-terminal to the most C-terminal cysteine C of Formula II.

In one embodiment, a polypeptide comprising the sequence of Formula II has a disulfide bond between the two Cs and has the conformation of X₅LX₇X₈ forming a type I beta turn structure with the center of the turn between L and X₇; and has a positive value for the dihedral angle phi of X₈. In another embodiment of Formula II, X₃ is an amino acid selected from the group consisting of M, A, V, L, I, Y, F, W and a non-polar amino acid. In another embodiment of Formula II, X₅ is selected from the group consisting of V, L, P, S, I, A and R. In another embodiment of Formula II, X₈ is selected from the group consisting of R, K, G, N, H and D-amino acid. In another embodiment of Formula II, X₉ is selected from the group consisting of H, K, A, R and Q.

In a further embodiment, the BAFF receptor from which the extracellular domain or BAFF-binding fragment or BAFF-binding variant thereof is derived is TACI, BR3 or BCMA. Alternatively, the BAFF antagonist can bind an extracellular domain of a native-sequence BR3 at its BAFF binding region to partially or fully block, inhibit or neutralize BAFF binding to BR3 in vitro, in situ, or in vivo. For example, such indirect antagonist is an anti-BR3 antibody that binds in a region of BR3 comprising residues 23-38 of human BR3 as defined below (SEQ ID NO:26) or a neighboring region of those residues such that binding of human BR3 to BAFF is sterically hindered.

In some embodiments, a BAFF antagonist according to this invention includes BAFF antibodies and immunoadhesins comprising an extracellular domain of a BAFF receptor, or fragments and variants thereof that bind native BAFF. In a further embodiment, the BAFF receptor from which the extracellular domain or BAFF-binding fragment or BAFF-binding variant thereof is derived is TACI, BR3 or BCMA. In a still another embodiment, the immunoadhesin comprises an amino acid sequence of that of Formula I or Formula II as set forth above, including an amino acid sequence selected from any one of the group consisting of SEQ ID NOS: 19, 20, 21, 22, 23, and 24.

According to one embodiment, the BAFF antagonist binds to a BAFF polypeptide or a BR3 polypeptide with a binding affinity of 100 nM or less. According to another embodiment, the BAFF antagonist binds to a BAFF polypeptide or a BR3 polypeptide with a binding affinity of 10 nM or less. According to yet another embodiment, the BAFF antagonist binds to a BAFF polypeptide or a BR3 polypeptide with a binding affinity of 1 nM or less.

The terms “BR3”, “BR3 polypeptide” or “BR3 receptor” when used herein encompass “native-sequence BR3 polypeptides” and “BR3 variants” (which are further defined herein). “BR3” is a designation given to those polypeptides comprising the following amino acid sequence and homologs thereof:

| human BR3 sequence (SEQ ID NO: 26): | |
|---|---|
| 1 | MRRGPRSLRGRDAPAPTPCVPAECFDLLVRHCVACGLLRTPRPKPAGASSPAPRTALQPQ |
| 61 | ESVGAGAGEAALPLPGLLFGAPALLGLALVLALVLVGLVSWRRRQRRLRGASSAEAPDGD |
| 121 | KDAPEPLDKVIILSPGISDATAPAWPPPGEDPGTTPPGHSVPVPATELGSTELVTTKTAG |
| 181 | PEQQ |

and variants or fragments thereof that bind native BAFF. The BR3 polypeptides of the invention can be isolated from a variety of sources, such as from human tissue types or from another source, or prepared by recombinant and/or synthetic methods. The term BR3 includes the BR3 polypeptides described in WO 2002/24909 and WO 2003/14294.

A “native-sequence” BR3 polypeptide or “native BR3” comprises a polypeptide having the same amino acid sequence as the corresponding BR3 polypeptide derived from nature. Such native-sequence BR3 polypeptides can be isolated from nature or can be produced by recombinant and/or synthetic means. The term “native-sequence BR3 polypeptide” specifically encompasses naturally occurring truncated, soluble or secreted forms (e.g., an extracellular domain sequence), naturally occurring variant forms (e.g., alternatively spliced forms) and naturally occurring allelic variants of the polypeptide. The BR3 polypeptides of the invention include the BR3 polypeptide comprising or consisting of the contiguous sequence of amino acid residues 1 to 184 of a human BR3 (SEQ ID NO:26).

A BR3 “extracellular domain” or “ECD” refers to a form of the BR3 polypeptide that is essentially free of the transmembrane and cytoplasmic domains. ECD forms of BR3 include a polypeptide comprising any one of the amino acid sequences selected from the group consisting of amino acids 1-77, 2-62, 2-71, 1-61, 7-71, 23-38 and 2-63 of human BR3. The invention contemplates BAFF antagonists that are polypeptides comprising any one of the above-mentioned ECD forms of human BR3 and variants and fragments thereof that bind a native BAFF.

Mini-BR3 is a 26-residue core region of the BAFF-binding domain of BR3, i.e., the amino acid sequence:

| | |
|---|---|
| TPCVPAECFD LLVRHCVACG LLRTPR | (SEQ ID NO: 27) |

“BR3 variant” means a BR3 polypeptide having at least about 80% amino acid sequence identity with the amino acid sequence of a native-sequence, full-length BR3 or BR3 ECD and binds a native-sequence BAFF polypeptide. Optionally, the BR3 variant includes a single cysteine-rich domain. Such BR3 variant polypeptides include, for instance, BR3 polypeptides wherein one or more amino acid residues are added, or deleted, at the N- and/or C-terminus, as well as within one or more internal domains, of the full-length amino acid sequence. Fragments of the BR3 ECD that bind a native sequence BAFF polypeptide are also contemplated. According to one embodiment, a BR3 variant polypeptide will have at least about 80% amino acid sequence identity, at least about 81% amino acid sequence identity, at least about 82% amino acid sequence identity, at least about 83% amino acid sequence identity, at least about 84% amino acid sequence identity, at least about 85% amino acid sequence identity, at least about 86% amino acid sequence identity, at least about 87% amino acid sequence identity, at least about 88% amino acid sequence identity, at least about 89% amino acid sequence identity, at least about 90% amino acid sequence identity, at least about 91% amino acid sequence identity, at least about 92% amino acid sequence identity, at least about 93% amino acid sequence identity, at least about 94% amino acid sequence identity, at least about 95% amino acid sequence identity, at least about 96% amino acid sequence identity, at least about 97% amino acid sequence identity, at least about 98% amino acid sequence identity or at least about 99% amino acid sequence identity with a human BR3 polypeptide or a specified fragment thereof (e.g., ECD). BR3 variant polypeptides do not encompass the native BR3 polypeptide sequence. According to another embodiment, BR3 variant polypeptides are at least about 10 amino acids in length, at least about 20 amino acids in length, at least about 30 amino acids in length, at least about 40 amino acids in length, at least about 50 amino acids in length, at least about 60 amino acids in length, or at least about 70 amino acids in length.

In one preferred embodiment, the BAFF antagonists herein are immunoadhesins comprising a portion of BR3, TACI or BCMA that binds BAFF, or variants thereof that bind BAFF. In other embodiments, the BAFF antagonist is a BAFF antibody. A “BAFF antibody” is an antibody that binds BAFF, and preferably binds BAFF within a region of human BAFF comprising residues 162-275 of the human BAFF sequence disclosed herein under the “BAFF” definition (SEQ ID NO:16). In another embodiment, the BAFF antagonist is BR3 antibody. A “BR3 antibody” is an antibody that binds BR3, and is preferably one that binds BR3 within a region of human BR3 comprising residues 23-38 of the human BR3 sequence disclosed herein under the “BR3” definition (SEQ ID NO:26). In general, the amino acid positions of human BAFF and human BR3 referred to herein are according to the sequence numbering under human BAFF and human BR3, SEQ ID NOS: 16 and 26, respectively, disclosed herein under the “BAFF” and “BR3” definitions.

Other examples of BAFF-binding polypeptides or BAFF antibodies can be found in, e.g., WO 2002/092620, WO 2003/014294, Gordon et al., Biochemistry 42(20):5977-5983 (2003), Kelley et al. J Biol. Chem. 279(16):16727-16735 (2004), WO 1998/18921, WO 2001/12812, WO 2000/68378 and WO 2000/40716.

A “package insert” is used to refer to instructions customarily included in commercial packages of therapeutic products, that contain information about the indications, usage, dosage, administration, contraindications, other therapeutic products to be combined with the packaged product, and/or warnings concerning the use of such therapeutic products, etc.

A “medicament” is an active drug to treat the Sjögren's syndrome or its symptoms or side effects.

A “Visual Analogue Scale” or “VAS” is a measurement of a characteristic or attitude that is believed to range across a continuum of values and cannot easily be directly measured. For example, the amount of pain that a patient feels ranges across a continuum from none to an extreme amount of pain. From the patient's perspective this spectrum appears continuous because the patient's pain does not take discrete jumps, as a categorization of none, mild, moderate and severe would suggest. It was to capture this idea of an underlying continuum that the VAS was devised. As such an assessment is clearly highly subjective, these scales are of most value when looking at change within individuals, and are of less value for comparing across a group of individuals at one time point. Hence, improvement over baseline on a VAS herein refers to improvement of the individual patient over his/her own baseline measurement on such a scale before treatment. In one embodiment, operationally a VAS is a horizontal line, 100 mm in length, anchored by word descriptors at each end. The patient marks on the line the point that the patient feels represents his or her perception of his or her current state. The VAS score is often determined by measuring in millimetres from the left-hand end of the line to the point that the patient marks. There are many other ways in which VAS scales have been presented, including vertical lines and lines with extra descriptors. Wewers & Lowe, Research in Nursing and Health 13: 227-236 (1990) provide an informative discussion of VAS. See also Gould et al., Journal of Clinical Nursing, 10: 697-706 (2001). The marker of dryness on such scale is dryness of eyes or mouth or a combination thereof as would occur as a symptom of Sjögren's syndrome. The marker of fatigue on such scale is fatigue characterized by loss of strength and energy, weariness, tiredness, and other forms of fatigue as a symptom of Sjögren's syndrome, and the marker of joint pain on such scale is joint pain or arthralgia affecting one or more joints that would occur as a symptom of Sjögren's syndrome, such as would occur with arthritis, for example, stiffening or inflammation of a joint such as the knee, knuckles, wrists, ankles, etc.

II. Treatment

In one aspect, the present invention provides a method of treating Sjögren's syndrome in a patient eligible for treatment, comprising administering an effective amount of an antagonist, preferably an antibody, that binds to a B-cell surface marker (more preferably a CD20 antibody) and an anti-malarial agent to the patient to provide at least about 30% (preferably at least about 35-50%) improvement over baseline in the patient in two or more of the following three measurements: dryness, fatigue, and joint pain, on a VAS. More preferably, the patient exhibits improvement from baseline in dryness and at least one of joint pain or fatigue. Preferably, the anti-malarial agent is hydroxychloroquine and no other medication, such as a steroid, is given.

In a preferred embodiment, the improvement over baseline is in all three of dryness, fatigue, and joint pain. Also, preferably the effective amount provides improvement over a control treatment comprising administering the anti-malarial agent to a patient but without the CD20 antibody.

The preferred anti-malarial agent is hydroxychloroquine or chloroquine, most preferably hydroxychloroquine.

In another embodiment, the present invention provides a method of treating Sjögren's syndrome in a subject eligible for treatment, comprising administering an effective amount of an antibody that binds to a B-cell surface marker (preferably a CD20 antibody) to the subject to provide an initial antibody exposure (of preferably about 0.5 to 4 grams, more preferably about 1.5 to 3.5 grams, and still more preferably about 1.5 to 2.5 grams) followed by a second antibody exposure (of preferably about 0.5 to 4 grams, more preferably about 1.5 to 3.5 grams, still more preferably about 1.5 to 2.5 grams), the second exposure not being provided until from about 16 to 54 weeks (preferably from about 20 to 30 weeks, more preferably from about 46 to 54 weeks) from the initial exposure. For purposes of this invention, the second antibody exposure is the next time the subject is treated with the CD20 antibody after the initial antibody exposure, there being no intervening CD20 antibody treatment or exposure between the initial and second exposures. Treatment includes, for example, meeting one or more primary and/or secondary efficacy endpoints as set forth in the Examples herein.

The method preferably comprises administering to the subject an effective amount of the CD20 antibody to provide a third antibody exposure (preferably of about 0.5-4 grams, more preferably about 1.5-3.5, still more preferably about 1.5-2.5 grams), the third exposure not being provided until from about 46 to 60 weeks (preferably about 46 to 55, more preferably about 46 to 52 weeks) from the initial exposure. Preferably, no further antibody exposure is provided until at least about 70-75 weeks from the initial exposure, and still more preferably no further antibody exposure is provided until about 74-80 weeks from the initial exposure.

Any one or more of the antibody exposures herein may be provided to the subject as a single dose of antibody, or as separate doses, for example, about 1-4 separate doses of the antibody (e.g, constituting a first and second dose, or a first, second, and third dose, or a first, second, third, and fourth dose, etc). The particular number of doses (whether one, two or three or more) employed for each antibody exposure is dependent, for example, on the type of Sjögren's syndrome treated, the type of antibody employed, whether, what type, and how much and how many of a second medicament is employed as noted below, and the method and frequency of administration. Where separate doses are administered, the later dose (for example, second or third dose) is preferably administered from about 1 to 20 days, more preferably from about 6 to 16 days, and most preferably from about 14 to 16 days from the time the previous dose was administered. The separate doses are preferably administered within a total period of between about 1 day and 4 weeks, more preferably between about 1 and 20 days (e.g., within a period of 6-18 days). In one such aspect, the separate doses are administered about weekly, with the second dose being administered about one week from the first dose and any third or subsequent dose being administered about one week from the second dose. Each such separate dose of the antibody is preferably about 0.5 to 1.5 grams, more preferably about 0.75 to 1.3 grams.

In one embodiment, the subject is provided at least about three exposures of the antibody, for example, from about 3 to 60 exposures, and more particularly about 3 to 40 exposures, most particularly, about 3 to 20 exposures. Preferably, such exposures are administered at intervals each of 24 weeks. In one embodiment, each antibody exposure is provided as a single dose of the antibody. In an alternative embodiment, each antibody exposure is provided as separate doses of the antibody. However, not every antibody exposure need be provided as a single dose or as separate doses.

In one preferred embodiment, about 2-3 grams of the CD20 antibody is administered as the initial exposure. If about 3 grams are administered, then about 1 gram of the CD20 antibody is administered weekly for about three weeks as the initial exposure. If about 2 grams of the CD20 antibody is administered as the initial exposure, then about 1 gram of the CD20 antibody is administered followed in about two weeks by another about 1 gram of the antibody as the initial exposure. In a preferred aspect, the second exposure is at about six months from the initial exposure and is administered in an amount of about 2 grams. In an alternative preferred aspect, the second exposure is at about six months from the initial exposure and is administered as about 1 gram of the antibody followed in about two weeks by another about 1 gram of the antibody. Preferably, an anti-malarial agent is administered to the subject along with the CD20 antibody. Additionally or alternatively, a steroid such as a corticosteroid is preferably administered with the initial antibody exposure. In a preferred aspect, the steroid is not administered with the second exposure or is administered with the second exposure but in lower amounts than are used with the initial exposure. Also preferred is wherein the steroid is not administered with third or later exposures.

In all the inventive methods set forth herein, the CD20 or B-cell surface marker antibody may be a naked antibody or may be conjugated with another molecule such as a cytotoxic agent such as a radioactive compound. The preferred CD20 antibody herein is a chimeric, humanized, or human CD20 antibody, more preferably rituximab, humanized 2H7 (e.g. comprising the variable domain sequences in SEQ ID Nos. 2 and 8), chimeric or humanized A20 antibody (Immunomedics), and HUMAX-CD20™ human CD20 antibody (Genmab). Still more preferred is rituximab or humanized 2H7.

Also, while the Sjögren's syndrome can be at any stage, in one preferred embodiment, the Sjögren's syndrome is secondary Sjögren's syndrome. In another preferred embodiment, the Sjögren's syndrome is primary Sjögren's syndrome.

In one embodiment, the subject has never been previously treated with drug(s), such as immunosuppressive agent(s), to treat the Sjögren's syndrome and/or has never been previously treated with an antibody to a B-cell surface marker (e.g. never been previously treated with a CD20 antibody). In a still further aspect, the patient has relapsed with the syndrome. In another embodiment, the patient has not relapsed with the syndrome. In another embodiment, the subject has been previously treated with drug(s) to treat the syndrome and/or has been previously treated with such antibody. In another embodiment, the CD20 antibody is the only medicament administered to the subject to treat the syndrome. In another embodiment, the CD20 antibody is one of the medicaments used to treat the syndrome. In a further embodiment, the subject does not have rheumatoid arthritis. In a still further embodiment, the subject does not have multiple sclerosis. In a yet further embodiment, the subject does not have lupus or ANCA-associated vasculitis. In yet another embodiment, the subject does not have an autoimmune disease other than Sjögren's syndrome. For purposes of this lattermost statement, an “autoimmune disease” herein is a disease or disorder arising from and directed against an individual's own tissues or organs or a co-segregate or manifestation thereof or resulting condition therefrom. In one embodiment, it refers to a condition that results from, or is aggravated by, the production by B cells of antibodies that are reactive with normal body tissues and antigens. In other embodiments, the autoimmune disease is one that involves secretion of an autoantibody that is specific for an epitope from a self antigen (e.g. a nuclear antigen).

In any of the methods herein, one may administer another medicament, in an effective amount, with the antagonist or antibody that binds a B-cell surface marker (e.g. with the CD20 antibody), such as a cytotoxic agent, chemotherapeutic agent, immunosuppressive agent, cytokine, cytokine antagonist or antibody, growth factor, hormone, integrin, integrin antagonist or antibody. In the first method herein wherein an anti-malarial agent is also employed, such medicament is called a third medicament, wherein the antagonist such as CD20 antibody (or combination of antagonists, e.g. antibodies) is a first medicament and the anti-malarial agent is a second medicament. In the second method herein wherein the antibody is administered in multiple exposures, such medicament is called a second medicament, wherein the antibody is a first medicament.

Examples of such additional medicaments include a chemotherapeutic agent, an interferon class drug such as interferon-alpha (e.g., from Amarillo Biosciences, Inc.), IFN-beta-1a (REBIF® and AVONEX®) or IFN-beta-1b (BETASERON®), an oligopeptide such as glatiramer acetate (COPAXONE®), a cytotoxic agent (such as mitoxantrone (NOVANTRONE®), methotrexate, cyclophosphamide, chlorambucil, and azathioprine), piroxicam (FELDENE®), a non-steroidal, anti-inflammatory medication possessing analgesic and antipyretic properties, intravenous immunoglobulin (gamma globulin), lymphocyte-depleting therapy (e.g., mitoxantrone, cyclophosphamide, CAMPATH™ antibodies, anti-CD4, cladribine, a polypeptide construct with at least two domains comprising a de-immunized, autoreactive antigen or its fragment that is specifically recognized by the Ig receptors of autoreactive B-cells (WO 2003/68822), total body irradiation, bone marrow transplantation), integrin antagonist or antibody (e.g., an LFA-1 antibody such as efalizumab/RAPTIVA® commercially available from Genentech, or an alpha 4 integrin antibody such as natalizumab/ANTEGREN® available from Biogen, or others as noted above), drugs that treat symptoms secondary or related to Sjögren's syndrome (e.g., dryness, swelling, incontinence, pain, fatigue) such as those noted herein, steroid such as corticosteroid (e.g., methylprednisolone, prednisone such as low-dose prednisone, dexamethasone, or glucocorticoid, e.g., via joint injection, including systemic corticosteroid therapy), non-lymphocyte-depleting immunosuppressive therapy (e.g., MMF or cyclosporine), cholesterol-lowering drug of the “statin” class (which includes cerivastatin (BAYCOL™), fluvastatin (LESCOL™), atorvastatin (LIPITOR™), lovastatin (MEVACOR™), pravastatin (PRAVACHOL™), and simvastatin (ZOCOR™)), estradiol, testosterone (optionally at elevated dosages; Stuve et al. Neurology 8:290-301 (2002)), androgen, hormone-replacement therapy, a TNF inhibitor, which may be useful at least in treating fatigue or other symptoms of the syndrome, DMARD such as an anti-malarial agent including those set forth above, NSAID, plasmapheresis, levothyroxine, cyclosporin A, somatastatin analogue, cytokine, anti-cytokine antagonist or antibody, anti-metabolite, immunosuppressive agent, rehabilitative surgery, radioiodine, thyroidectomy, BAFF antagonist such as BAFF or BR3 antibodies or immunoadhesins, anti-CD40 receptor or anti-CD40 ligand (CD154), anti-IL-6 receptor antagonist/antibody, another B-cell surface antagonist or antibody such as humanized 2H7 or other humanized or human CD20 antibody with rituximab, etc. Such additional medicament also includes other types of treatments such as gene therapy, for example, human gene transfer studies for head and neck cancer treatment of patients to repair damaged salivary glands due to Sjögren's syndrome.

More specific examples of such medicaments include a moisture-replacement therapy such as eye drops to ease, for example, the symptoms of dryness, chemotherapeutic agent, a cytotoxic agent, anti-integrin, gamma globulin, anti-CD4, cladribine, corticosteroid, MMF, cyclosporine, cholesterol-lowering drug of the statin class, estradiol, testosterone, androgen, hormone-replacement drug, a TNF inhibitor, DMARD, NSAID (to treat, for example musculoskeletal symptoms), levothyroxine, cyclosporin A, somatastatin analogue, cytokine antagonist or cytokine-receptor antagonist, anti-metabolite, anti-malarial agent, BAFF antagonist such as BAFF antibody or BR3 antibody, especially a BAFF antibody, immunosuppressive agent, and another B-cell surface marker antibody, such as a combination of rituximab and humanized 2H7 or other humanized CD20 antibody.

The more preferred such medicaments are a chemotherapeutic agent, an immunosuppressive agent, a BAFF antagonist such as a BAFF or BR3 antibody, a DMARD, moisture replacement therapy, a cytotoxic agent, an integrin antagonist, a NSAID, a cytokine antagonist, a secretory agonist, or a hormone, or a combination thereof, more preferably a steroid, a secretory agonist for dry mouth or dry eye, a NSAID, or an immunosuppressive agent, or a combination thereof. A DMARD such as anti-malarial agents may be useful, for example, for relief of joint pains, skin rashes, and hair loss. Steroids may be required, for example, in some subjects with more severe complications such as vasculitis or nervous system involvement, and with organ-threatening disease (e.g., when NSAIDS and anti-malarial agents have failed), including steroids such as corticosteroids, e.g., prednisone, methylprednisolone, hydrocortisone, or dexamethasone. Secretory agonists such as SALAGEN® pilocarpine hydrochloride, EVOXAC® cevimeline, or bromhexine or pharmaceutical salts thereof are useful as second medicaments to treat for dry mouth, for example, and diquafosol, REFRESH ENDURA® lubricant eye drops, cevimeline, cysteamine eye drops, and cyclosporine ophthalmic emulsion to treat dry eye. In addition, NSAIDs are useful, for example, for relief of joint pains, swelling, muscle ache, fever, and include aspirin, naproxen, ibuprofen, indomethacin, and tolmetin. Immunosuppressants may be required, for example, for very active disease with major organ involvement, and include such agents as cyclophosphamide (CYTOXAN®), chlorambucil, azathioprine (IMURAN®), and methotrexate. BAFF antagonists may be useful in combination with the CD20 antibody for efficacy.

Still more preferred are DMARDs, NSAIDs, and for more severe complications, a corticosteroid, chemotherapeutic agent, an immunosuppressive agent, a cytotoxic agent, an integrin antagonist, a cytokine antagonist, or a hormone, most preferably a NSAID, a corticosteroid, or an immunosuppressive agent. For the second medicament, also preferred is an anti-malarial agent, alone or with another second medicament.

In one particularly preferred embodiment, the second or third medicament is or comprises a steroid, for example, a corticosteroid, which is preferably prednisone, methylprednisolone, hydrocortisone, or dexamethasone. Such steroid is preferably administered in lower amounts than are used if the CD20 antibody is not administered to a patient treated with steroid.

In another particularly preferred aspect, the second or third medicament is a secretory agonist for dry mouth, more preferably pilocarpine hydrochloride, cevimeline, or bromhexine or pharmaceutical salts thereof, or for dry eye (for example, diquafosol, cysteamine eye drops, REFRESH ENDURA® lubricant eye drops, cevimeline, and cyclosporine ophthalmic emulsion).

In an alternatively particularly preferred embodiment, the second or third medicament is a NSAID, more preferably aspirin, naproxen, ibuprofen, indomethacin, or tolmetin.

In a still further particularly preferred aspect, the second or third medicament is an immunosuppressive agent, more preferably cyclophosphamide, chlorambucil, azathioprine, or methotrexate.

All these second or third medicaments may be used in combination with each other or by themselves with the CD20 antibody, so that the expression “second medicament” or “third medicament” as used herein does not mean it is the only medicament besides the first or second medicament, respectively. Thus, the second or third medicament need not be one medicament, but may constitute or comprise more than one such drug.

These second and third medicaments as set forth herein are generally used in the same dosages and with administration routes as used hereinbefore or about from 1 to 99% of the heretofore-employed dosages. If such second or third medicaments are used at all, preferably, they are used in lower amounts than if the CD20 antibody were not present, especially in subsequent dosings beyond the initial dosing with antibody, so as to eliminate or reduce side effects caused thereby.

Where a second medicament is administered in an effective amount with an antibody exposure, it may be administered with any exposure, for example, only with one exposure, or with more than one exposure. In one embodiment, the second medicament is administered with the initial exposure. In another embodiment, the second medicament is administered with the initial and second exposures. In a still further embodiment, the second medicament is administered with all exposures. It is preferred that after the initial exposure, such as of steroid, the amount of such agent is reduced or eliminated so as to reduce the exposure of the subject to an agent with side effects such as prednisone and cyclophosphamide.

The combined administration of a second and/or third medicament includes co-administration (concurrent administration), using separate formulations or a single pharmaceutical formulation, and consecutive administration in either order, wherein preferably there is a time period while both (or all) active agents (medicaments) simultaneously exert their biological activities.

The antibody or antagonist herein is administered by any suitable means, including parenteral, topical, subcutaneous, intraperitoneal, intrapulmonary, intranasal, and/or intralesional administration. Parenteral infusions include intramuscular, intravenous, intraarterial, intraperitoneal, or subcutaneous administration. Intrathecal administration is also contemplated (see, e.g., US 2002/0009444, Grillo-Lopez, A concerning intrathecal delivery of a CD20 antibody). In addition, the antibody or antagonist may suitably be administered by pulse infusion, e.g., with declining doses of the antibody or antagonist. Preferably, the dosing is given intravenously or subcutaneously, and more preferably by intravenous infusion(s).

If multiple exposures of antibody are provided, each exposure may be provided using the same or a different administration means. In one embodiment, each exposure is by intravenous administration. In another embodiment, each exposure is given by subcutaneous administration. In yet another embodiment, the exposures are given by both intravenous and subcutaneous administration.

In one embodiment, the CD20 antibody is administered as a slow intravenous infusion rather than an intravenous push or bolus. For example, a steroid such as prednisolone or methylprednisolone (e.g., about 80-120 mg i.v., more specifically about 100 mg i.v.) is administered about 30 minutes prior to any infusion of the CD20 antibody. The CD20 antibody is, for example, infused through a dedicated line.

For the initial dose of a multi-dose exposure to CD20 antibody, or for the single dose if the exposure involves only one dose, such infusion is preferably commenced at a rate of about 50 mg/hour. This may be escalated, e.g., at a rate of about 50 mg/hour increments every about 30 minutes to a maximum of about 400 mg/hour. However, if the subject is experiencing an infusion-related reaction, the infusion rate is preferably reduced, e.g., to half the current rate, e.g., from 100 mg/hour to 50 mg/hour. Preferably, the infusion of such dose of CD20 antibody (e.g., an about 1000-mg total dose) is completed at about 255 minutes (4 hours 15 min.). Optionally, the subjects receive a prophylactic treatment of acetaminophen/paracetamol (e.g., about 1 g) and diphenhydramine HCl (e.g., about 50 mg or, equivalent dose of similar agent) by mouth about 30 to 60 minutes prior to the start of an infusion.

If more than one infusion (dose) of CD20 antibody is given to achieve the total exposure, the second or subsequent CD20 antibody infusions in this infusion embodiment are preferably commenced at a higher rate than the initial infusion, e.g., at about 100 mg/hour. This rate may be escalated, e.g., at a rate of about 100 mg/hour increments every about 30 minutes to a maximum of about 400 mg/hour. Subjects who experience an infusion-related reaction preferably have the infusion rate reduced to half that rate, e.g., from 100 mg/hour to 50 mg/hour. Preferably, the infusion of such second or subsequent dose of CD20 antibody (e.g., an about 1000-mg total dose) is completed by about 195 minutes (3 hours 15 minutes).

A discussion of methods of producing, modifying, and formulating such antibodies follows.

III. Production of Antibodies

The methods and articles of manufacture of the present invention use, or incorporate, an antibody that binds to a B-cell surface marker, especially one that binds to CD20. Accordingly, methods for generating such antibodies will be described here.

CD20 antigen to be used for production of, or screening for, antibody(ies) may be, e.g., a soluble form of CD20 or a portion thereof, containing the desired epitope. Alternatively, or additionally, cells expressing CD20 at their cell surface can be used to generate, or screen for, antibody(ies). Other forms of CD20 useful for generating antibodies will be apparent to those skilled in the art.

A description follows as to exemplary techniques for the production of the antibodies used in accordance with the present invention.

(i) Polyclonal Antibodies

Polyclonal antibodies are preferably raised in animals by multiple subcutaneous (sc) or intraperitoneal (ip) injections of the relevant antigen and an adjuvant. It may be useful to conjugate the relevant antigen to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl₂, or R¹N═C═NR, where R and R¹ are different alkyl groups.

Animals are immunized against the antigen, immunogenic conjugates, or derivatives by combining, e.g., 100 μg or 5 μg of the protein or conjugate (for rabbits or mice, respectively) with 3 volumes of Freund's complete adjuvant and injecting the solution intradermally at multiple sites. One month later the animals are boosted with ⅕ to 1/10 the original amount of peptide or conjugate in Freund's complete adjuvant by subcutaneous injection at multiple sites. Seven to 14 days later the animals are bled and the serum is assayed for antibody titer. Animals are boosted until the titer plateaus. Preferably, the animal is boosted with the conjugate of the same antigen, but conjugated to a different protein and/or through a different cross-linking reagent. Conjugates also can be made in recombinant cell culture as protein fusions. Also, aggregating agents such as alum are suitably used to enhance the immune response.

(ii) Monoclonal Antibodies

Monoclonal antibodies are obtained from a population of substantially homogeneous antibodies, i.e., the individual antibodies comprising the population are identical and/or bind the same epitope except for possible variants that arise during production of the monoclonal antibody, such variants generally being present in minor amounts. Thus, the modifier “monoclonal” indicates the character of the antibody as not being a mixture of discrete or polyclonal antibodies.

For example, the monoclonal antibodies may be made using the hybridoma method first described by Kohler et al., Nature, 256:495 (1975), or may be made by recombinant DNA methods (U.S. Pat. No. 4,816,567).

In the hybridoma method, a mouse or other appropriate host animal, such as a hamster, is immunized as hereinabove described to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)).

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk Institute Cell Distribution Center, San Diego, Calif. USA, and SP-2 or X63-Ag8-653 cells available from the American Type Culture Collection, Rockville, Md. USA. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunoabsorbent assay (ELISA).

The binding affinity of the monoclonal antibody can, for example, be determined by the Scatchard analysis of Munson et al., Anal. Biochem., 107:220 (1980).

After hybridoma cells are identified that produce antibodies of the desired specificity, affinity, and/or activity, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce immunoglobulin protein, to obtain the synthesis of monoclonal antibodies in the recombinant host cells. Review articles on recombinant expression in bacteria of DNA encoding the antibody include Skerra et al., Curr. Opinion in Immunol., 5:256-262 (1993) and Plückthun, Immunol. Revs., 130:151-188 (1992).

In a further embodiment, antibodies or antibody fragments can be isolated from antibody phage libraries generated using the techniques described in McCafferty et al., Nature, 348:552-554 (1990). Clackson et al., Nature, 352:624-628 (1991) and Marks et al., J. Mol. Biol., 222:581-597 (1991) describe the isolation of murine and human antibodies, respectively, using phage libraries. Subsequent publications describe the production of high affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al., Nuc. Acids. Res., 21:2265-2266 (1993)). Thus, these techniques are viable alternatives to traditional monoclonal antibody hybridoma techniques for isolation of monoclonal antibodies.

The DNA also may be modified, for example, by substituting the coding sequence for human heavy- and light chain constant domains in place of the homologous murine sequences (U.S. Pat. No. 4,816,567; Morrison, et al., Proc. Natl. Acad. Sci. USA, 81:6851 (1984)), or by covalently joining to the immunoglobulin coding sequence all or part of the coding sequence for a non-immunoglobulin polypeptide.

Typically such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

(iii) Humanized Antibodies

Methods for humanizing non-human antibodies have been described in the art. Preferably, a humanized antibody has one or more amino acid residues introduced into it from a source that is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting hypervariable region sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some hypervariable region residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence that is closest to that of the rodent is then accepted as the human framework region (FR) for the humanized antibody (Sims et al., J. Immunol., 151:2296 (1993); Chothia et al., J. Mol. Biol., 196:901 (1987)). Another method uses a particular framework region derived from the consensus sequence of all human antibodies of a particular subgroup of light or heavy chain variable regions. The same framework may be used for several different humanized antibodies (Carter et al., Proc. Natl. Acad. Sci. USA, 89:4285 (1992); Presta et al., J. Immunol., 151:2623 (1993)).

It is further important that antibodies be humanized with retention of high affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available that illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

(iv) Human Antibodies

As an alternative to humanization, human antibodies can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy chain joining region (J_{H}) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-258 (1993); Bruggermann et al., Year in Immuno., 7:33 (1993); and U.S. Pat. Nos. 5,591,669, 5,589,369 and 5,545,807.

Alternatively, phage display technology (McCafferty et al., Nature 348:552-553 (1990)) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B cell. Phage display can be performed in a variety of formats; for their review see, e.g., Johnson, Kevin S. and Chiswell, David J., Current Opinion in Structural Biology 3:564-571 (1993). Several sources of V-gene segments can be used for phage display. Clackson et al., Nature, 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., J. Mol. Biol. 222:581-597 (1991), or Griffith et al., EMBO J. 12:725-734 (1993). See, also, U.S. Pat. Nos. 5,565,332 and 5,573,905.

Human antibodies may also be generated by in vitro activated B cells (see U.S. Pat. Nos. 5,567,610 and 5,229,275).

(v) Antibody Fragments

Various techniques have been developed for the production of antibody fragments. Traditionally, these fragments were derived via proteolytic digestion of intact antibodies (see, e.g., Morimoto et al., Journal of Biochemical and Biophysical Methods 24:107-117 (1992) and Brennan et al., Science, 229:81 (1985)). However, these fragments can now be produced directly by recombinant host cells. For example, the antibody fragments can be isolated from the antibody phage libraries discussed above. Alternatively, Fab′-SH fragments can be directly recovered from E. coli and chemically coupled to form F(ab′)₂ fragments (Carter et al., Bio/Technology 10:163-167 (1992)). According to another approach, F(ab′)₂ fragments can be isolated directly from recombinant host cell culture. Other techniques for the production of antibody fragments will be apparent to the skilled practitioner. In other embodiments, the antibody of choice is a single chain Fv fragment (scFv). See WO 93/16185; U.S. Pat. No. 5,571,894; and U.S. Pat. No. 5,587,458. The antibody fragment may also be a “linear antibody”, e.g., as described in U.S. Pat. No. 5,641,870 for example. Such linear antibody fragments may be monospecific or bispecific.

(vi) Bispecific Antibodies

Bispecific antibodies are antibodies that have binding specificities for at least two different epitopes. Exemplary bispecific antibodies may bind to two different epitopes of the CD20 antigen. Other such antibodies may bind CD20 and further bind a second B-cell surface marker. Alternatively, an anti-CD20 binding arm may be combined with an arm that binds to a triggering molecule on a leukocyte such as a T-cell receptor molecule (e.g. CD2 or CD3), or Fc receptors for IgG (FcγR), such as FcγRI (CD64), FcγRII (CD32) and FcγRIII (CD16) so as to focus cellular defense mechanisms to the B cell. Bispecific antibodies may also be used to localize cytotoxic agents to the B cell. These antibodies possess a CD20-binding arm and an arm that binds the cytotoxic agent (e.g. saporin, anti-interferon-α, vinca alkaloid, ricin A chain, methotrexate or radioactive isotope hapten). Bispecific antibodies can be prepared as full length antibodies or antibody fragments (e.g. F(ab′)₂ bispecific antibodies).

Methods for making bispecific antibodies are known in the art. Traditional production of full length bispecific antibodies is based on the coexpression of two immunoglobulin heavy chain-light chain pairs, where the two chains have different specificities (Millstein et al., Nature, 305:537-539 (1983)). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., EMBO J., 10:3655-3659 (1991).

According to a different approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2, and CH3 regions. It is preferred to have the first heavy chain constant region (CH1) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., Methods in Enzymology, 121:210 (1986).

According to another approach described in U.S. Pat. No. 5,731,168, the interface between a pair of antibody molecules can be engineered to maximize the percentage of heterodimers that are recovered from recombinant cell culture. The preferred interface comprises at least a part of the CH3 domain of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al., Science, 229: 81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Various techniques for making and isolating bispecific antibody fragments directly from recombinant cell culture have also been described. For example, bispecific antibodies have been produced using leucine zippers. Kostelny et al., J. Immunol., 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA, 90:6444-6448 (1993) has provided an alternative mechanism for making bispecific antibody fragments. The fragments comprise a heavy chain variable domain (V_{H}) connected to a light chain variable domain (V_{L}) by a linker that is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See Gruber et al., J. Immunol., 152:5368 (1994).

Antibodies with more than two valencies are contemplated. For example, trispecific antibodies can be prepared. Tutt et al. J. Immunol. 147: 60 (1991).

IV. Conjugates and Other Modifications of the Antibody

The antibody used in the methods or included in the articles of manufacture herein is optionally conjugated to a cytotoxic agent. For instance, the (CD20) antibody may be conjugated to a drug as described in WO2004/032828.

Chemotherapeutic agents useful in the generation of such antibody-cytotoxic agent conjugates have been described above.

Conjugates of an antibody and one or more small molecule toxins, such as a calicheamicin, a maytansine (U.S. Pat. No. 5,208,020), a trichothene, and CC1065 are also contemplated herein. In one embodiment of the invention, the antibody is conjugated to one or more maytansine molecules (e.g. about 1 to about 10 maytansine molecules per antibody molecule). Maytansine may, for example, be converted to May-SS-Me, which may be reduced to May-SH3 and reacted with modified antibody (Chari et al. Cancer Research 52: 127-131 (1992)) to generate a maytansinoid-antibody conjugate.

Alternatively, the antibody is conjugated to one or more calicheamicin molecules. The calicheamicin family of antibiotics are capable of producing double-stranded DNA breaks at sub-picomolar concentrations. Structural analogues of calicheamicin that may be used include, but are not limited to, γ₁^{I}, α₂^{I}, α₃^{I}, N-acetyl-β₁^{I}, PSAG and θ^{I}₁ (Hinman et al. Cancer Research 53: 3336-3342 (1993) and Lode et al. Cancer Research 58: 2925-2928 (1998)).

Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin and the tricothecenes. See, for example, WO 93/21232 published Oct. 28, 1993.

The present invention further contemplates antibody conjugated with a compound with nucleolytic activity (e.g. a ribonuclease or a DNA endonuclease such as a deoxyribonuclease; DNase).

A variety of radioactive isotopes are available for the production of radioconjugated antibodies. Examples include At²¹¹, I¹³¹, I¹²⁵, Y⁹⁰, Re¹⁸⁶, Re¹⁸⁸, Sm¹⁵³, Bi²¹², P³² and radioactive isotopes of Lu.

Conjugates of the antibody and cytotoxic agent may be made using a variety of bifunctional protein coupling agents such as N-succinimidyl-3-(2-pyridyldithiol)propionate (SPDP), succinimidyl-4-(N-maleimidomethyl) cyclohexane-1-carboxylate, iminothiolane (IT), bifunctional derivatives of imidoesters (such as dimethyl adipimidate HCl), active esters (such as disuccinimidyl suberate), aldehydes (such as glutareldehyde), bis-azido compounds (such as bis(p-azidobenzoyl)hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al. Science 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of the cytotoxic drug in the cell. For example, an acid-labile linker, peptidase-sensitive linker, dimethyl linker or disulfide-containing linker (Chari et al. Cancer Research 52: 127-131 (1992)) may be used.

Alternatively, a fusion protein comprising the antibody and cytotoxic agent may be made, e.g. by recombinant techniques or peptide synthesis.

In yet another embodiment, the antibody may be conjugated to a “receptor” (such streptavidin) for utilization in tumor pretargeting wherein the antibody-receptor conjugate is administered to the subject, followed by removal of unbound conjugate from the circulation using a clearing agent and then administration of a “ligand” (e.g. avidin) that is conjugated to a cytotoxic agent (e.g. a radionucleotide).

The antibodies of the present invention may also be conjugated with a prodrug-activating enzyme that converts a prodrug (e.g. a peptidyl chemotherapeutic agent, see WO81/01145) to an active anti-cancer drug. See, for example, WO 88/07378 and U.S. Pat. No. 4,975,278.

The enzyme component of such conjugates includes any enzyme capable of acting on a prodrug in such a way so as to covert it into its more active, cytotoxic form.

Enzymes that are useful in the method of this invention include, but are not limited to, alkaline phosphatase useful for converting phosphate-containing prodrugs into free drugs; arylsulfatase useful for converting sulfate-containing prodrugs into free drugs; cytosine deaminase useful for converting non-toxic 5-fluorocytosine into the anti-cancer drug, 5-fluorouracil; proteases, such as serratia protease, thermolysin, subtilisin, carboxypeptidases and cathepsins (such as cathepsins B and L), that are useful for converting peptide-containing prodrugs into free drugs; D-alanylcarboxypeptidases, useful for converting prodrugs that contain D-amino acid substituents; carbohydrate-cleaving enzymes such as β-galactosidase and neuraminidase useful for converting glycosylated prodrugs into free drugs; β-lactamase useful for converting drugs derivatized with β-lactams into free drugs; and penicillin amidases, such as penicillin V amidase or penicillin G amidase, useful for converting drugs derivatized at their amine nitrogens with phenoxyacetyl or phenylacetyl groups, respectively, into free drugs. Alternatively, antibodies with enzymatic activity, also known in the art as “abzymes”, can be used to convert the prodrugs of the invention into free active drugs (see, e.g., Massey, Nature 328: 457-458 (1987)). Antibody-abzyme conjugates can be prepared as described herein for delivery of the abzyme to a tumor cell population.

The enzymes of this invention can be covalently bound to the antibody by techniques well known in the art such as the use of the heterobifunctional crosslinking reagents discussed above. Alternatively, fusion proteins comprising at least the antigen binding region of an antibody of the invention linked to at least a functionally active portion of an enzyme of the invention can be constructed using recombinant DNA techniques well known in the art (see, e.g., Neuberger et al., Nature, 312: 604-608 (1984)).

Other modifications of the antibody are contemplated herein. For example, the antibody may be linked to one of a variety of nonproteinaceous polymers, e.g., polyethylene glycol (PEG), polypropylene glycol, polyoxyalkylenes, or copolymers of polyethylene glycol and polypropylene glycol. Antibody fragments, such as Fab′, linked to one or more PEG molecules are an especially preferred embodiment of the invention.

The antibodies disclosed herein may also be formulated as liposomes. Liposomes containing the antibody are prepared by methods known in the art, such as described in Epstein et al., Proc. Natl. Acad. Sci. USA, 82:3688 (1985); Hwang et al., Proc. Natl. Acad. Sci. USA, 77:4030 (1980); U.S. Pat. Nos. 4,485,045 and 4,544,545; and WO97/38731 published Oct. 23, 1997. Liposomes with enhanced circulation time are disclosed in U.S. Pat. No. 5,013,556.

Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. Fab′ fragments of an antibody of the present invention can be conjugated to the liposomes as described in Martin et al. J. Biol. Chem. 257: 286-288 (1982) via a disulfide interchange reaction. A chemotherapeutic agent is optionally contained within the liposome. See Gabizon et al. J. National Cancer Inst. 81(19)1484 (1989).

Amino acid sequence modification(s) of protein or peptide antibodies described herein are contemplated. For example, it may be desirable to improve the binding affinity and/or other biological properties of the antibody. Amino acid sequence variants of the antibody are prepared by introducing appropriate nucleotide changes into the antibody nucleic acid, or by peptide synthesis. Such modifications include, for example, deletions from, and/or insertions into and/or substitutions of, residues within the amino acid sequences of the antibody. Any combination of deletion, insertion, and substitution is made to arrive at the final construct, provided that the final construct possesses the desired characteristics. The amino acid changes also may alter post-translational processes of the antibody, such as changing the number or position of glycosylation sites.

A useful method for identification of certain residues or regions of the antibody that are preferred locations for mutagenesis is called “alanine scanning mutagenesis” as described by Cunningham and Wells Science, 244:1081-1085 (1989). Here, a residue or group of target residues are identified (e.g., charged residues such as arg, asp, his, lys, and glu) and replaced by a neutral or negatively charged amino acid (most preferably alanine or polyalanine) to affect the interaction of the amino acids with antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, ala scanning or random mutagenesis is conducted at the target codon or region and the expressed antibody variants are screened for the desired activity.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue or the antibody fused to a cytotoxic polypeptide. Other insertional variants of the antibody molecule include the fusion to the N- or C-terminus of the antibody of an enzyme, or a polypeptide that increases the serum half-life of the antibody.

Another type of variant is an amino acid substitution variant. These variants have at least one amino acid residue in the antibody molecule replaced by different residue. The sites of greatest interest for substitutional mutagenesis of antibodies include the hypervariable regions, but FR alterations are also contemplated. Conservative substitutions are shown in Table 1 under the heading of “preferred substitutions”. If such substitutions result in a change in biological activity, then more substantial changes, denominated “exemplary substitutions” in Table 1, or as further described below in reference to amino acid classes, may be introduced and the products screened.

TABLE 1

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; | Leu |
| | Phe; Norleucine | |
| Leu (L) | Norleucine; Ile; Val; | Ile |
| | Met; Ala; Phe | |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; | Leu |
| | Ala; Norleucine | |

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Amino acids may be grouped according to similarities in the properties of their side chains (in A. L. Lehninger, in Biochemistry, second ed., pp. 73-75, Worth Publishers, New York (1975)):

(1) non-polar: Ala (A), Val (V), Leu (L), Ile (I), Pro (P), Phe (F), Trp (W), Met (M)

(2) uncharged polar: Gly (G), Ser (S), Thr (T), Cys (C), Tyr (Y), Asn (N), Gln (Q)

(3) acidic: Asp (D), Glu (E)

(4) basic: Lys (K), Arg (R), His (H)

Alternatively, naturally occurring residues may be divided into groups based on common side-chain properties:

(1) hydrophobic: Norleucine, Met, Ala, Val, Leu, Ile;

(2) neutral hydrophilic: Cys, Ser, Thr, Asn, Gln;

(3) acidic: Asp, Glu;

(4) basic: His, Lys, Arg;

(5) residues that influence chain orientation: Gly, Pro; (6) aromatic: Trp, Tyr, Phe.

Non-conservative substitutions will entail exchanging a member of one of these classes for another class.

Any cysteine residue not involved in maintaining the proper conformation of the antibody also may be substituted, generally with serine, to improve the oxidative stability of the molecule and prevent aberrant crosslinking. Conversely, cysteine bond(s) may be added to the antibody to improve its stability (particularly where the antibody is an antibody fragment such as an Fv fragment).

A particularly preferred type of substitutional variant involves substituting one or more hypervariable region residues of a parent antibody. Generally, the resulting variant(s) selected for further development will have improved biological properties relative to the parent antibody from which they are generated. A convenient way for generating such substitutional variants is affinity maturation using phage display. Briefly, several hypervariable region sites (e.g. 6-7 sites) are mutated to generate all possible amino substitutions at each site. The antibody variants thus generated are displayed in a monovalent fashion from filamentous phage particles as fusions to the gene III product of M13 packaged within each particle. The phage-displayed variants are then screened for their biological activity (e.g. binding affinity) as herein disclosed. In order to identify candidate hypervariable region sites for modification, alanine scanning mutagenesis can be performed to identify hypervariable region residues contributing significantly to antigen binding. Alternatively, or in additionally, it may be beneficial to analyze a crystal structure of the antigen-antibody complex to identify contact points between the antibody and antigen. Such contact residues and neighboring residues are candidates for substitution according to the techniques elaborated herein. Once such variants are generated, the panel of variants is subjected to screening as described herein and antibodies with superior properties in one or more relevant assays may be selected for further development.

Another type of amino acid variant of the antibody alters the original glycosylation pattern of the antibody. Such altering includes deleting one or more carbohydrate moieties found in the antibody, and/or adding one or more glycosylation sites that are not present in the antibody.

Glycosylation of polypeptides is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

Where the antibody comprises an Fc region, the carbohydrate attached thereto may be altered. For example, antibodies with a mature carbohydrate structure that lacks fucose attached to an Fc region of the antibody are described in US Pat Appl No US 2003/0157108 (Presta, L.). See also US 2004/0093621 (Kyowa Hakko Kogyo Co., Ltd). Antibodies with a bisecting N-acetylglucosamine (GlcNAc) in the carbohydrate attached to an Fc region of the antibody are referenced in WO 2003/011878, Jean-Mairet et al. and U.S. Pat. No. 6,602,684, Umana et al. Antibodies with at least one galactose residue in the oligosaccharide attached to an Fc region of the antibody are reported in WO 1997/30087, Patel et al. See, also, WO 1998/58964 (Raju, S.) and WO 1999/22764 (Raju, S.) concerning antibodies with altered carbohydrate attached to the Fc region thereof.

The preferred glycosylation variant herein comprises an Fc region, wherein a carbohydrate structure attached to the Fc region lacks fucose. Such variants have improved ADCC function. Optionally, the Fc region further comprises one or more amino acid substitutions therein which further improve ADCC, for example, substitutions at positions 298, 333, and/or 334 of the Fc region (Eu numbering of residues). Examples of publications related to “defucosylated” or “fucose-deficient” antibodies include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; Okazaki et al. J. Mol. Biol. 336:1239-1249 (2004); Yamane-Ohnuki et al. Biotech. Bioeng. 87: 614 (2004). Examples of cell lines producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (Yamane-Ohnuki et al. Biotech. Bioeng. 87: 614 (2004)).

Nucleic acid molecules encoding amino acid sequence variants of the antibody are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the antibody.

It may be desirable to modify the antibody of the invention with respect to effector function, e.g. so as to enhance antigen-dependent cell-mediated cyotoxicity (ADCC) and/or complement dependent cytotoxicity (CDC) of the antibody. This may be achieved by introducing one or more amino acid substitutions in an Fc region of an antibody. Alternatively or additionally, cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al., J. Exp Med. 176:1191-1195 (1992) and Shopes, B. J. Immunol. 148:2918-2922 (1992). Homodimeric antibodies with enhanced anti-tumor activity may also be prepared using heterobifunctional cross-linkers as described in Wolff et al. Cancer Research 53:2560-2565 (1993). Alternatively, an antibody can be engineered that has dual Fc regions and may thereby have enhanced complement lysis and ADCC capabilities. See Stevenson et al. Anti-Cancer Drug Design 3:219-230 (1989).

WO00/42072 (Presta, L.) describes antibodies with improved ADCC function in the presence of human effector cells, where the antibodies comprise amino acid substitutions in the Fc region thereof. Preferably, the antibody with improved ADCC comprises substitutions at positions 298, 333, and/or 334 of the Fc region. Preferably the altered Fc region is a human IgG1 Fc region comprising or consisting of substitutions at one, two or three of these positions.

Antibodies with altered C1q binding and/or complement dependent cytotoxicity (CDC) are described in WO99/51642, U.S. Pat. No. 6,194,551B1, U.S. Pat. No. 6,242,195B1, U.S. Pat. No. 6,528,624B1 and U.S. Pat. No. 6,538,124 (Idusogie et al.). The antibodies comprise an amino acid substitution at one or more of amino acid positions 270, 322, 326, 327, 329, 313, 333 and/or 334 of the Fc region thereof.

To increase the serum half-life of the antibody, one may incorporate a salvage receptor binding epitope into the antibody (especially an antibody fragment) as described in U.S. Pat. No. 5,739,277, for example. As used herein, the term “salvage receptor binding epitope” refers to an epitope of the Fc region of an IgG molecule (e.g., IgG₁, IgG₂, IgG₃, or IgG₄) that is responsible for increasing the in vivo serum half-life of the IgG molecule. Antibodies with substitutions in an Fc region thereof and increased serum half-lives are also described in WO00/42072 (Presta, L.).

Engineered antibodies with three or more (preferably four) functional antigen binding sites are also contemplated (US Appln No. US2002/0004587 A1, Miller et al.).

V. Pharmaceutical Formulations

Therapeutic formulations of the antbodies used in accordance with the present invention are prepared for storage by mixing an antibody having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

Exemplary anti-CD20 antibody formulations are described in WO98/56418. This publication describes a liquid multidose formulation comprising 40 mg/mL rituximab, 25 mM acetate, 150 mM trehalose, 0.9% benzyl alcohol, 0.02% polysorbate 20 at pH 5.0 that has a minimum shelf life of two years storage at 2-8° C. Another anti-CD20 formulation of interest comprises 10 mg/mL rituximab in 9.0 mg/mL sodium chloride, 7.35 mg/mL sodium citrate dihydrate, 0.7 mg/mL polysorbate 80, and Sterile Water for Injection, pH 6.5.

Lyophilized formulations adapted for subcutaneous administration are described in U.S. Pat. No. 6,267,958 (Andya et al.). Such lyophilized formulations may be reconstituted with a suitable diluent to a high protein concentration and the reconstituted formulation may be administered subcutaneously to the mammal to be treated herein.

Crystallized forms of the antibody are also contemplated. See, for example, US 2002/0136719A1 (Shenoy et al.).

The formulation herein may also contain more than one active compound (a second or third medicament as noted above) as necessary, preferably those with complementary activities that do not adversely affect each other. The type and effective amounts of such medicaments depend, for example, on the amount of antibody present in the formulation, and clinical parameters of the subjects. The preferred such medicaments are noted above.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPO™ (injectable microspheres composed of lactic acid-glycolic acid copolymer and leuprolide acetate), and poly-D-(−)-3-hydroxybutyric acid.

The formulations to be used for in vivo administration must be sterile. This is readily accomplished by filtration through sterile filtration membranes.

VI. Articles of Manufacture

In another embodiment of the invention, articles of manufacture containing materials useful for the treatment of Sjögren's syndrome described above are provided.

In one aspect, the article of manufacture comprises (a) a container comprising an antagonist that binds to a B-cell surface marker (e.g., an antibody that so binds, including a CD20 antibody) (preferably the container comprises the antagonist or antibody and a pharmaceutically acceptable carrier or diluent within the container); (b) a container comprising an anti-malarial agent (preferably the container comprises the anti-malarial agent and a pharmaceutically acceptable carrier or diluent within the container); and (c) a package insert with instructions for treating Sjögren's syndrome in a patient, wherein the instructions indicate that amounts of the antibody or antagonist and the anti-malarial agent are administered to the patient that are effective to provide at least an about 30% improvement over baseline in two or more of dryness, fatigue, and joint pain on a Visual Analogue Scale.

In a preferred embodiment, the article of manufacture herein further comprises a container comprising a third medicament, wherein the antagonist or antibody is a first medicament and the anti-malarial agent is a second medicament, and which article further comprises instructions on the package insert for treating the patient with the third medicament, in an effective amount. The third medicament may be any of those set forth above, with an exemplary third medicament being a chemotherapeutic agent, an immunosuppressive agent, a cytotoxic agent, an integrin antagonist, a cytokine antagonist, or a hormone. The preferred third medicaments are those set forth above, and most preferred is a steroid.

In another aspect, the invention provides an article of manufacture comprising: (a) a container comprising an antibody that binds to a B-cell surface marker (e.g., a CD20 antibody) (preferably the container comprises the antibody and a pharmaceutically acceptable carrier or diluent within the container); and (b) a package insert with instructions for treating Sjögren's syndrome in a subject, wherein the instructions indicate that an amount of the antibody is administered to the subject that is effective to provide an initial antibody exposure followed by a second antibody exposure, wherein the second exposure is not provided until from about 16 to 54 weeks from the initial exposure.

Preferably, such package insert is provided with instructions for treating Sjögren's syndrome in a subject, wherein the instructions indicate that an amount of the antibody is administered to the subject that is effective to provide an initial antibody exposure of about 0.5 to 4 grams followed by a second antibody exposure of about 0.5 to 4 grams, wherein the second exposure is not provided until from about 16 to 54 weeks from the initial exposure and each of the antibody exposures is provided to the subject as about one to four doses, preferably as a single dose or as two or three separate doses of antibody.

In a preferred embodiment of this inventive aspect, the article of manufacture herein further comprises a container comprising a second medicament, wherein the CD20 antibody is a first medicament, and which article further comprises instructions on the package insert for treating the subject with the second medicament, in an effective amount. The second medicament may be any of those set forth above, with an exemplary second medicament being a chemotherapeutic agent, an immunosuppressive agent, a cytotoxic agent, an integrin antagonist, a cytokine antagonist, or a hormone, most preferably an anti-malarial agent. In another preferred embodiment of this inventive aspect, the article of manufacture herein further comprises a container comprising a third medicament, with instructions on the package insert for treating the subject with the third medicament. Preferably, such third medicament is those that are mentioned above as preferred, and most preferably a steroid.

In all of these aspects, the package insert is on or associated with the container. Suitable containers include, for example, bottles, vials, syringes, etc. The containers may be formed from a variety of materials such as glass or plastic. The container holds or contains a composition that is effective for treating the Sjögren's syndrome and may have a sterile access port (for example the container may be an intravenous solution bag or a vial having a stopper pierceable by a hypodermic injection needle). At least one active agent in the composition is the antagonist or antibody. The label or package insert indicates that the composition is used for treating Sjögren's syndrome in a patient or subject eligible for treatment with specific guidance regarding dosing amounts and intervals of antagonist or antibody and any other medicament being provided. The article of manufacture may further comprise an additional container comprising a pharmaceutically acceptable diluent buffer, such as bacteriostatic water for injection (BWFI), phosphate-buffered saline, Ringer's solution, and/or dextrose solution. The article of manufacture may further include other materials desirable from a commercial and user standpoint, including other buffers, diluents, filters, needles, and syringes.

Further details of the invention are illustrated by the following non-limiting Examples. The disclosures of all citations in the specification are expressly incorporated herein by reference.

### EXAMPLE 1

### Study of Efficacy and Safety of Rituximab in Patients with Moderate-to-Severe Sjögren's Syndrome

This study assesses the superiority of efficacy and safety of rituximab (MABTHERA®/RITUXAN®) compared to placebo for acute treatment of signs and symptoms in patients with moderate-to-severe primary Sjögren's syndrome exhibiting one or more symptoms of systemic disease. The PvR is used to cut Sjögren's into primary Sjögren's patients. The ratio of primary to secondary Sjögren's syndrome is approximately 1:1, with Thomas et al. British J Rheumatol 1998; 37: 1069-76 (1998) indicating that the percent of primary Sjögren's is approximately 56% (95% CI, 45%-64%).

Rituximab (1000 mg i.v.×2) is administered i.v. in two initial doses at days 1 and 15 with i.v. hydrochloroquinone (HQ) plus steroids. This experimental regimen is compared to the same regimen except using rituximab placebo instead of rituximab, with 1:1 randomization between the two arms of the study, with about 48 patients per arm (total 96 patients). This rituximab-based regimen challenges the current standard of care, limits patient exposure to steroids and its known toxicities, and demonstrates improved net clinical benefit. Patients are monitored for disease activity, use of additional immunosuppressants, steroid usage and safety events over the trial length of one year, with the primary efficacy endpoint of the trial measured at 3 months with follow-up to I year. Safety follow-up is required until 12 months following the last dose of rituximab or return of B cells into the normal range, whichever occurs later.

The primary objective is to determine the proportion of patients achieving the primary efficacy endpoint, which is improvement in VAS (dryness, fatigue, joint pain) and no prespecified adverse event. Specifically, the primary endpoint is defined as improvement over baseline in 2 out of 3 VAS scale (dryness, fatigue, joint pain) of at least about 30% over baseline.

The secondary endpoints are salivary scintigraphy, Rose Bengal, individual VAS, TJC, SF-36, ESR, and hyperglobulinemia, as well as exploratory methods such as infiltrate, biopsy, MRI, and presence of anti-SSA/Ro/anti-SSB/La antibodies.

It is predicted and expected that administration of rituximab (or humanized 2H7 substituted for rituximab) to the patients in the protocol set forth above will ameliorate one or more signs, symptoms, or other indicators of Sjögren's syndrome over the control.

Phase II

In particular, for Phase II studies, the results at 3 months are expected to be as follows:

Expected Primary Endpoint:

A response rate in VAS (two of three of dryness, fatigue, joint pain): at least about 30% over baseline, preferably about 40 to >50%, more preferably about 50 to >60%, where expected placebo response is about 30%.

Expected Secondary Endpoints:

Salivary Flow: about 40 to >50% of patients will have a clinical response (expected placebo response of about 25%)

Schirmer's test: about 40 to >50% of patients will have a clinical response (expected placebo response is about 25%)

Tender Joint Count (TJC): about 40% to >50% of patients will have a clinical response (expected placebo response is about 30%)

MOS Short form-36 (SF-36): about 40% to >50% of patients will have a clinical response (expected placebo response is about 30%)

Erythrocyte sedimentation rate (ESR): about >40% to 50% of patients will have a clinical response (expected placebo response is about 20%)

Hyperglobulinemia: about 32% to >40% of patients will have a clinical response (expected placebo response is about 20%)

Exploratory endpoints: infiltrate/biopsy=about 30%; Ro/La autoantibodies

Infusion Reactions:

Severe about <1%-<5%, Non Fatal

Infections/SAE

No significant or manageable increase in infections or SAEs

HACA

about <3% to <12% with low clinical implications

Phase III

For phase III studies, the results at 6 months are expected to be as follows:

Expected Primary Endpoint

A response rate in VAS (two of three of dryness, fatigue, joint pain) or objective measurement from phase II: at least about 30% over baseline, preferably about 40 to >60% where expected placebo response is about 30%.

Secondary Endpoints:

Salivary Flow about 40 to >50% of patients will have a clinical response (placebo response rate about 25%)

Schirmer's test: about 40 to >50% of patients will have a clinical response (placebo response rate about 25%)

Plus 1-2 of following, depending on Phase II outcomes:

Salivary scintigraphy about 40 to >50% of patients will have a clinical response (placebo response rate about 25%)

TJC: about 40 to >50% of patients will have a clinical response (placebo response rate about 30%)

SF-36: about 40 to >50% of patients will have a clinical response (placebo response rate about 30%)

ESR: about 40 to >50% of patients will have a clinical response (placebo response rate about 20%)

Hyperglobulinemia: about 32 to >40% of patients will have a clinical response (placebo response rate about 20%)

Rose Bengal: about 40 to >50% of patients will have a clinical response (placebo response rate about 25%)

Exploratory endpoints: infiltrate/biopsy/MRI=about 30%; Ro/La autoantibodies, assessment of specific organ involvement, e.g., vasculitis, lung, kidney

Infusion Reactions

Severe about <1-<5%, Non Fatal

Infections/SAE

No significant increase or manageable increase in infections or SAEs HACA

about <3% to <12% with low clinical implications

### EXAMPLE 2

### Retreatment Study of Efficacy and Safety of Rituximab in Patients with Moderate-to-Severe Sjögren's Syndrome

This study assesses the superiority of efficacy and safety of rituximab (MABTHERA®/RITUXAN®) compared to placebo in adult subjects with moderate-to-severe primary Sjögren's syndrome. Rituximab (1000 mg i.v.×3) is administered i.v. in three initial doses at days 1, 8, and 15 with i.v. hydrochloroquine (HQ) and prednisone, followed by 1 g×2 at six months. This experimental regimen is compared to rituximab placebo+the same doses of HQ and prednisone. This rituximab-based regimen challenges the current standard of care, and is expected to demonstrate improved net clinical benefit. Patients are monitored for disease activity, use of additional immunosuppressants, flares of disease, prednisone usage and safety events over the 50 weeks of the study. The primary efficacy endpoint of the trial is at 50 weeks, and efficacy measures are assessed by a unique Examining Assessor who is not involved with patient treatment or other study procedures. Safety follow-up is required until 12 months following the last dose of rituximab or return of B cells into the normal range, whichever occurs later.

The primary objective is to determine the proportion of patients achieving a primary endpoint and no prespecified adverse event. A primary endpoint is obtaining at least 30% improvement over the subject's baseline in two or more of dryness, fatigue, and joint pain on a VAS. See Example I for expected and preferred Phase II and III primary and secondary endpoints for use in this trial.

The experimental arm receives the first i.v. rituximab/placebo infusion of 1000 mg on day 0 with the second infusion occurring on day 8 and the third infusion on day 15. These subjects also receive 2 initial doses of i.v. prednisone and HQ (750 mg/m²) on days 3 and 18. All subjects receive a second rituximab/placebo infusion course of 1000 mg i.v. separated by 14 days at weeks 24 and 26, respectively.

B-cell counts (CD19) are assessed at baseline, at the end of each course of rituximab/placebo, and every 4 weeks thereafter throughout the study. All B-cell counts will be conducted at the sponsor-assigned central laboratory. B-cell depletion is defined as ≦5 CD19+B cells/μl or ≧95% depletion of CD19+B cells from baseline value at screening. At the end of 50 weeks, subjects who received placebo rituximab or active rituximab but demonstrate B-cell recovery will complete study participation. Subjects who received rituximab but have not demonstrated B-cell recovery will be followed for 12 months after the last course of rituximab or until B-cell recovery, whichever occurs first. Sites will be informed as to whether a subject must continue in follow-up but not whether the subject received placebo or rituximab.

Subjects who reach the primary endpoint of confirmed clinical response without a prespecified adverse event at week 50 receive cyclosphosphamide given at month 14 and 17 or placebo i.v. HQ. All subjects, including those who discontinue, will be observed for 50 weeks after their last rituximab/placebo infusion or until their B-cell counts recover.

The primary outcome of this study is to determine the proportion of subjects able to be effectively and safely re-treated with rituximab.

A dose of 1000 mg rituximab or placebo equivalent is administered i.v. at days 0, 8 and 15, and again at weeks 24 and 26. Subjects that experience a new or recurrent flare of disease on baseline immunosuppressive therapy are enrolled. Baseline immuno-suppression may include anti-malarial agent, prednisone, hydroxychloroquine, methotrexate, azathioprine or MMF. Baseline medications such as MTX; AZA or MMF are discontinued at trial entry to prevent over-immunosuppression. Subjects that have received cyclosphosphamide therapy within the 3 months prior to enrollment will be excluded.

It is predicted and expected that administration of rituximab or humanized 2H7 to the subject in the protocol set forth above will ameliorate one or more signs, symptoms, or other indicators of Sjögren's syndrome over the control. It is also expected that at about week 48-54, another 2-g dose of the CD20 antibody given all at once or spread out over about 14-16 days in 1-gram amounts would be effective to treat Sjögren's syndrome for the entire second year, with or without the prednisone and/or other immunosuppressive agents. Thus, the CD20 antibody would be administered initially within about the 2-week time period, followed by another treatment at about 4-8 months, followed by another treatment at about one year from initial treatment (measured from the time any one of the doses was given), followed by treatment at about two years from initial treatment, with expected success, in about one-gram×2-4 dosing for each treatment, administered together, about weekly, or about every other week over about two to four weeks. This re-treatment protocol is expected to be successfully used for many years with little or no adverse effects.

### EXAMPLE 3

### A Retreatment Study to Evaluate the Efficacy and Safety of Rituximab in Subjects with Moderate-to-Severe Systemic Sjögren's Syndrome

This study assesses the efficacy and safety of rituximab (MABTHERA®/RITUXAN®) added to prednisone and HQ compared with placebo in subjects with moderate-to-severe primary Sjögren's syndrome at enrollment for a Phase II/III trial. Subjects are randomized at week 2 to receive rituximab and HQ and prednisone or placebo. Subjects are monitored for disease activity, use of additional immunosuppressants, flares of disease, prednisone use, and safety events over the 50 weeks of the study. The primary efficacy endpoint of the trial will be at 50 weeks, and efficacy measures are assessed by a unique Examining Assessor who is not involved with patient treatment or other study procedures. Safety follow-up is required until 12 months following the last dose of rituximab or return of B cells into the normal range, whichever occurs later.

The primary objective is to investigate the efficacy of rituximab relative to placebo to improve signs, symptoms or other indicators in subjects with Sjögren's syndrome over 50 weeks. See Example 1 for expected and preferred Phase II and III primary and secondary endpoints for use in this trial.

Consented subjects participate in a screening period lasting up to 14 days to determine eligibility. Subjects are treated with oral prednisone 0.4 mg/kg/day to 1.0 mg/kg/day for 28 days. Eligible subjects are randomized in a 1:1 ratio to receive rituximab 1000 mg i.v.×2 (days 1, 15) plus prednisone and HQ during the 50-week treatment and observation period. The first rituximab/placebo infusion occurs on Day 0 with the second infusion occurring on Day 15+/−1 day. Changes in immunosuppressive drugs are not permitted during the study, unless mandated by toxicity, and requests to taper an immunosuppressive drug must be discussed in advance with the Medical Monitor. For all subjects in the absence of increasing disease activity, a subsequent course of rituximab or placebo infusions is administered at weeks 24 and 26 and consists of 2 biweekly doses. Courses of rituximab treatment must be separated by a minimum interval of 16 weeks.

Patients are assessed monthly for 12 months. B-cell counts are assessed at baseline, at the end of each course of rituximab/placebo infusion, and subsequently every 4 weeks throughout the treatment/observation period. All B-cell counts are performed by a central laboratory, and physicians will be blinded to B-cell counts. B-cell depletion is defined as ≦5 CD19+B cells/μl or ≧95% depletion of CD19+B cells from baseline value at screening. At the end of 50 weeks, subjects who received rituximab placebo or rituximab but demonstrate B-cell recovery will complete study participation. Subjects who received rituximab but have not demonstrated B-cell recovery at 50 weeks are observed for 6 months following the last course of rituximab or until B-cell recovery, whichever occurs first. The primary efficacy outcome measure is the time-adjusted area under the curve minus baseline of BILAG score at week 50.

A dose of 1000 mg rituximab or placebo equivalent is administered i.v. on day 0 and day 15. Study personnel will be trained on how to properly administer rituximab. Subjects may be hospitalized for observation, particularly for their first infusion, at the discretion of the investigator. Rituximab must be administered under close supervision, and full resuscitation facilities must be immediately available.

It is predicted and expected that administration of rituximab or humanized 2H7 to the subject in the protocol set forth above will ameliorate one or more signs, symptoms, or other indicators of Sjögren's syndrome over the control. It is also expected that at about week 48-54, another 2-g dose of the CD20 antibody given all at once or spread out over about 14-16 days in 1-gram amounts would be effective to treat Sjögren's syndrome for the entire second year, with or without the prednisone and/or other immunosuppressive agents. Thus, the CD20 antibody would be administered initially within about the 2-week time period, followed by another treatment at about 4-8 months, followed by another treatment at about one year from initial treatment (measured from the time any one of the doses was given), followed by treatment at about two years from initial treatment, with expected success, in about one-gram×2-4 dosing for each treatment, administered together, about weekly, or about every other week over about two to four weeks. This re-treatment protocol is expected to be successfully used for many years with little or no adverse effects.

In addition, it is expected that the CD20 antibody will be effective for treating patients with less severe symptoms such as those with mild systemic primary Sjögren's syndrome, where the primary endpoint would be at least 30% improvement over baseline of one or more of fatigue, chronic pain, or dryness on a VAS and/or the patient is not on any concomitant medication such as a hydroxychloroquine and/or steroid before treatment and/or does not need to be placed on such medication during the treatment with CD20 antibody. The expected and preferred primary and secondary Phase II and Phase III endpoints noted in Example 1 would be used for such trial, except that for the primary endpoint, only one of the VAS factors need be improved to indicate efficacy.

### EXAMPLE 4

### A Separate Retreatment Study to Evaluate the Efficacy and Safety of Rituximab in Subjects with Moderate-to-Severe Systemic SjÖgren's Syndrome

It is expected that Example 3 results would be successful if the same types of patients were initially treated with rituximab and then re-treated with rituximab one year after first being treated, using the same dosing and other protocol of Example 3 except that rituximab is given at one-year intervals rather than six-month intervals. The same or similar results would be expected for patients with less severe symptoms as noted above.

### EXAMPLE 5

### Humanized 2H7 Variants Useful Herein

Useful for purposes herein are humanized 2H7 antibodies comprising one, two, three, four, five, or six of the following CDR sequences:

CDR L1 sequence RASSSVSYXH wherein X is M or L (SEQ ID NO:18), for example, SEQ ID NO:4 (FIG. 1A),

CDR L2 sequence of SEQ ID NO:5 (FIG. 1A),

CDR L3 sequence QQWXFNPPT wherein X is S or A (SEQ ID NO:19), for example, SEQ ID NO:6 (FIG. 1A),

CDR H1 sequence of SEQ ID NO:10 (FIG. 1B),

CDR H2 sequence of AIYPGNGXTSYNQKFKG wherein X is D or A (SEQ ID NO:20), for example, SEQ ID NO:11 (FIG. 1B), and

CDR H3 sequence of VVYYSXXYWYFDV wherein the X at position 6 is N, A, Y, W, or D, and the X at position 7 is S or R (SEQ ID NO:21), for example, SEQ ID NO:12 (FIG. 1B).

The CDR sequences above are generally present within human variable light- and variable heavy-framework sequences, such as substantially the human consensus FR residues of human light-chain kappa subgroup I (V_{L}κI), and substantially the human consensus FR residues of human heavy-chain subgroup III (V_{H}III). See also WO 2004/056312 (Lowman et al.).

The variable heavy region may be joined to a human IgG chain constant region, wherein the region may be, for example, IgG1 or IgG3, including native-sequence and non-native-sequence constant regions.

In a preferred embodiment, such antibody comprises the variable heavy-domain sequence of SEQ ID NO:8 (v16, as shown in FIG. 1B), optionally also comprising the variable light-domain sequence of SEQ ID NO:2 (v16, as shown in FIG. 1A), which optionally comprises one or more amino acid substitution(s) at positions 56, 100, and/or 100a, e.g., D56A, N100A, or N100Y, and/or S100aR in the variable heavy domain and one or more amino acid substitution(s) at positions 32 and/or 92, e.g. M32L and/or S92A, in the variable light domain. Preferably, the antibody is an intact antibody comprising the light-chain amino acid sequence of SEQ ID NO:13 or 16, and heavy-chain amino acid sequence of SEQ ID NO:14, 15, 17, or 22, where SEQ ID NO:22 is indicated below.

A preferred humanized 2H7 antibody is ocrelizumab (Genentech, Inc.).

The antibody herein may further comprise at least one amino acid substitution in the Fc region that improves ADCC activity, such as one wherein the amino acid substitutions are at positions 298, 333, and 334, preferably S298A, E333A, and K334A, using Eu numbering of heavy-chain residues. See also U.S. Pat. No. 6,737,056, L. Presta.

Any of these antibodies may comprise at least one substitution in the Fc region that improves FcRn binding or serum half-life, for example, a substitution at heavy-chain position 434, such as N434W. See also U.S. Pat. No. 6,737,056, L. Presta.

Any of these antibodies may further comprise at least one amino acid substitution in the Fc region that increases CDC activity, for example, comprising at least a substitution at position 326, preferably K326A or K326W. See also U.S. Pat. No. 6,528,624, Idusogie et al.

Some preferred humanized 2H7 variants are those comprising the variable light domain of SEQ ID NO:2 and the variable heavy domain of SEQ ID NO:8, including those with or without substitutions in an Fc region (if present), and those comprising a variable heavy domain with alteration in SEQ ID NO:8 of N100A; or D56A and N100A; or D56A, N100Y, and S100aR; and a variable light domain with alteration in SEQ ID NO:2 of M32L; or S92A; or M32L and S92A.

M34 in the variable heavy domain of 2H7.v16 has been identified as a potential source of antibody stability and is another potential candidate for substitution.

In a summary of some various preferred embodiments of the invention, the variable region of variants based on 2H7.v16comprise the amino acid sequences of v16 except at the positions of amino acid substitutions that are indicated in Table 2 below. Unless otherwise indicated, the 2H7 variants will have the same light chain as that of v16.

TABLE 2
Exemplary Humanized 2H7 Antibody Variants

| 2H7 | Heavy chain | Light chain | |
|---|---|---|---|
| Version | (V_{H}) changes | (V_{L}) changes | Fc changes |
| 16 | | | — |
| for reference | | | |
| 31 | — | — | S298A, E333A, K334A |
| 73 | N100A | M32L | |
| 75 | N100A | M32L | S298A, E333A, K334A |
| 96 | D56A, N100A | S92A | |
| 114 | D56A, N100A | M32L, S92A | S298A, E333A, K334A |
| 115 | D56A, N100A | M32L, S92A | S298A, E333A, K334A, |
| | | | E356D, M358L |
| 116 | D56A, N100A | M32L, S92A | S298A, K334A, K322A |
| 138 | D56A, N100A | M32L, S92A | S298A, E333A, K334A, |
| | | | K326A |
| 477 | D56A, N100A | M32L, S92A | S298A, E333A, K334A, |
| | | | K326A, N434W |
| 375 | — | — | K334L |
| 588 | — | — | S298A, E333A, K334A, |
| | | | K326A |
| 511 | D56A, N100Y, | M32L, S92A | S298A, E333A, K334A, |
| | S100aR | | K326A |

One preferred humanized 2H7 comprises 2H7.v16 variable light-domain sequence:

| | |
|---|---|
| DIQMTQSPSSLSASVGDRVTITCRASSSVSYMHWYQQKPGKAPKPLIYAPSNLASGVPSRFSGSGSGTD | (SEQ ID NO: 2) |
| FTLTISSLQPEDFATYYCQQWSFNPPTFGQGTKVEIKR; | |

and 2H7.v16 variable heavy-domain sequence:

| | |
|---|---|
| EVQLVESGGGLVQPGGSLRLSCAASGYTFTSYNMHWVRQAPGKGLEWVGAIYPGNGDTSYNQKFKG | (SEQ ID NO: 8) |
| RFTISVDKSKNTLYLQMNSLRAEDTAVYYCARVVYYSNSYWYFDVWGQGTLVTVSS. | |

Where the humanized 2H7.v16 antibody is an intact antibody, it may comprise the light-chain amino acid sequence:

| | |
|---|---|
| DIQMTQSPSSLSASVGDRVTITCRASSSVSYMHWYQQKPGKAPKPLIYAPSNLASGVPSRFSGSGSGTD | (SEQ ID NO: 13) |
| FTLTISSLQPEDFATYYCQQWSFNPPTFGQGTKVEIKRTVAAPSVFIFPPSDEQLKSGTASVVCLLNNFYP | |
| REAKVQWKVDNALQSGNSQESVTEQDSKDSTYSLSSTLTLSKADYEKHKVYACEVTHQGLSSPVTKS | |
| FNRGEC; | |

and the heavy-chain amino acid sequence of SEQ ID NO:14 or:

| | |
|---|---|
| EVQLVESGGGLVQPGGSLRLSCAASGYTFTSYNMHWVRQAPGKGLEWVGAIYPGNGDTSYNQKFKG | (SEQ ID NO: 15) |
| RFTISVDKSKNTLYLQMNSLRAEDTAVYYCARVVYYSNSYWYFDVWGQGTLVTVSSASTKGPSVFPL | |
| APSSKSTSGGTAALGCLVKDYFPEPVTVSWNSGALTSGVHTFPAVLQSSGLYSLSSVVTVPSSSLGTQT | |
| YICNVNHKPSNTKVDKKVEPKSCDKTHTCPPCPAPELLGGPSVFLFPPKPKDTLMISRTPEVTCVVVDV | |
| SHEDPEVKFNWYVDGVEVHNAKTKPREEQYNSTYRVVSVLTVLHQDWLNGKEYKCKVSNKALPAPI | |
| EKTISKAKGQPREPQVYTLPPSREEMTKNQVSLTCLVKGFYPSDIAVEWESNGQPENNYKTTPPVLDSD | |
| GSFFLYSKLTVDKSRWQQGNVFSCSVMHEALHNHYTQKSLSLSPG. | |

Another preferred humanized 2H7 antibody comprises 2H7.v511 variable light-domain sequence:

| | |
|---|---|
| DIQMTQSPSSLSASVGDRVTITCRASSSVSYLHWYQQKPGKAPKPLIYAPSNLASGVPSRFSGSGSGTDF | (SEQ ID NO: 23) |
| TLTISSLQPEDFATYYCQQWAFNPPTFGQGTKVEIKR | |

and 2H7.v511 variable heavy-domain sequence:

| | |
|---|---|
| EVQLVESGGGLVQPGGSLRLSCAASGYTFTSYNMHWVRQAPGKGLEWVGAIYPGNGATSYN | (SEQ ID NO: 24) |
| QKFKGRFTISVDKSKNTLYLQMNSLRAEDTAVYYCARVVYYSYRYWYFDVWGQGTLVTVSS. | |

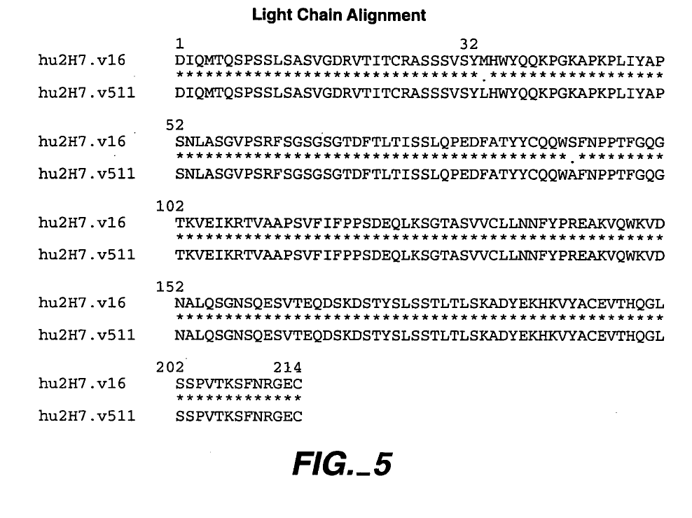

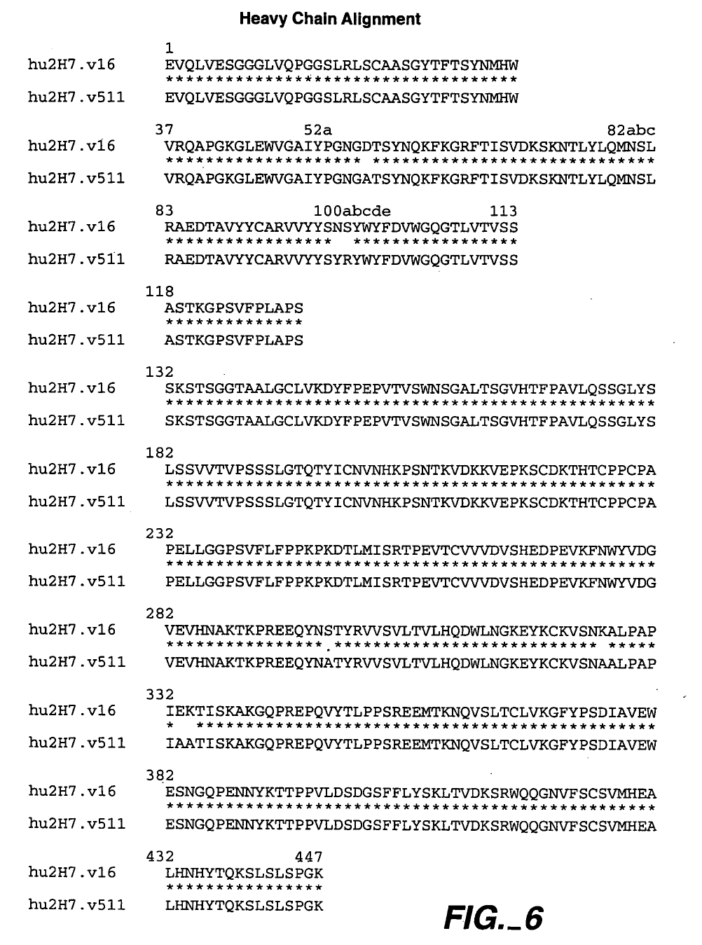

See FIGS. 5 and 6, which align the mature light and heavy chains, respectively, of humanized 2H7.v511 with humanized 2H7.v16.

Where the humanized 2H7.v31 antibody is an intact antibody, it may comprise the light-chain amino acid sequence:

| | |
|---|---|
| DIQMTQSPSSLSASVGDRVTITCRASSSVSYLHWYQQKPGKAPKPLIYAPSNLASGVPSRFSGSGSGTDF | (SEQ ID NO: 13) |
| TLTISSLQPEDFATYYCQQWAFNPPTFGQGTKVEIKRTVAAPSVFIFPPSDEQLKSGTASVVCLLNNFYP | |
| REAKVQWKVDNALQSGNSQESVTEQDSKDSTYSLSSTLTLSKADYEKHKVYACEVTHQGLSSPVTKS | |
| FNRGEC | |

and the heavy-chain amino acid sequence of SEQ ID NO:15 or:

| | |
|---|---|
| EVQLVESGGGLVQPGGSLRLSCAASGYTFTSYNMHWVRQAPGKGLEWVGAIYPGNGATSYNQKFKG | (SEQ ID NO: 22) |
| RFTISVDKSKNTLYLQMNSLRAEDTAVYYCARVVYYSYRYWYFDVWGQGTLVTVSSASTKGPSVFPL | |
| APSSKSTSGGTAALGCLVKDYFPEPVTVSWNSGALTSGVHTFPAVLQSSGLYSLSSVVTVPSSSLGTQT | |
| YICNVNHKPSNTKVDKKVEPKSCDKTHTCPPCPAPELLGGPSVFLFPPKPKDTLMISRTPEVTCVVVDV | |
| SHEDPEVKFNWYVDGVEVHNAKTKPREEQYNATYRVVSVLTVLHQDWLNGKEYKCKVSNAALPAPI | |
| AATISKAKGQPREPQVYThPPSREEMTKNQVSLTCLVKGFYPSDIAVEWESNGQPENNYKTTPPVLDSD | |
| GSFFLYSKLTVDKSRWQQGNVFSCSVMHEALHNHYTQKSLSLSPG. | |

A preferred embodiment herein is where the antibody is humanized 2H7 comprising the variable domain sequences in SEQ ID NOS:2 and 8. Another preferred embodiment herein is where the antibody is humanized 2H7 comprising the variable domain sequences in SEQ ID NOS:23 and 24.

What is claimed is:

1. A method of treating Sjögren's syndrome in a patient comprising administering an effective amount of a CD20 antibody and an anti-malarial agent to the patient to provide at least about 30% improvement over baseline in two or more of dryness, fatigue, and joint pain on a Visual Analogue Scale.

2. The method of claim 1 wherein the improvement over baseline is in three of dryness, fatigue, and joint pain.

3. The method of claim 1 wherein the effective amount provides improvement over a control treatment administering the anti-malarial agent without CD20 antibody.

4. The method of claim 1 wherein the anti-malarial agent is hydroxychloroquine or chloroquine.

5. The method of claim 1 wherein the anti-malarial agent is hydroxychloroquine.

6. The method of claim 1 wherein a third medicament is administered in an effective amount, wherein the CD20 antibody is a first medicament and the anti-malarial agent is a second medicament.

7. The method of claim 6 wherein the third medicament is a chemotherapeutic agent, an immunosuppressive agent, a disease-modifying anti-rheumatic drug (DMARD), a cytotoxic agent, an integrin antagonist, a nonsteroidal antiinflammatory drug (NSAID), a cytokine antagonist, a secretory agonist, or a hormone.

8. The method of claim 6 wherein the third medicament is a steroid, a secretory agonist for dry mouth or dry eye, a nonsteroidal antiinflammatory drug (NSAID), or an immunosuppressive agent.

9. The method of claim 6 wherein the third medicament is a steroid.

10. The method of claim 9 wherein the steroid is a corticosteroid.

11. The method of claim 10 wherein the steroid is prednisone, methylprednisolone, hydrocortisone, or dexamethasone.

12. The method of claim 9 wherein the steroid is administered in lower amounts than are used if the CD20 antibody is not administered to a patient treated with steroid.

13. The method of claim 6 wherein the third medicament is a secretory agonist for dry mouth or dry eye.

14. The method of claim 13 wherein the secretory agonist is pilocarpine hydrochloride, cevimeline, bromhexine, diquafosol, cysteamine eye drops, lubricant eye drops, cyclosporine ophthalmic emulsion, or pharmaceutical salts thereof.

15. The method of claim 6 wherein the third medicament is a nonsteroidal antiinflammatory drug (NSAID).

16. The method of claim 15 wherein the NSAID is aspirin, naproxen, ibuprofen, indomethacin, or tolmetin.

17. The method of claim 6 wherein the third medicament is an immunosuppressive agent.

18. The method of claim 17 wherein the immunosuppressive agent is cyclophosphamide, chlorambucil, azathioprine, or methotrexate.

19. The method of claim 1 wherein the patient has never been previously treated with a CD20 antibody.

20. The method of claim 1 wherein the patient has relapsed with the syndrome.

21. The method of claim 1 wherein the antibody is a naked antibody.

22. The method of claim 1 wherein the antibody is conjugated with another molecule.

23. The method of claim 22 wherein the other molecule is a cytotoxic agent.

24. The method of claim 1 wherein the antibody is administered intravenously.

25. The method of claim 1 wherein the antibody is administered subcutaneously.

26. The method of claim 1 wherein the antibody is rituximab.

27. The method of claim 1 wherein the antibody is humanized 2H7 comprising the variable domain sequences in SEQ ID Nos. 2 and 8.

28. The method of claim 1 wherein the patient has an elevated level of anti-nuclear antibodies (ANA), anti-rheumatoid factor (RF) antibodies, antibodies directed against Sjögren's-associated antigen A or B (SS-A or SS-B), antibodies directed against centromere protein B (CENP B) or centromere protein C (CENP C), an autoantibody to ICA69, or a combination of two or more of such antibodies.

29. The method of claim 28 wherein the antibodies directed against SS-A and SS-B are anti-Ro/SS-A antibodies, anti-La/SS-A antibodies, anti-La/SS-B antibodies, or anti-Ro/SS-B antibodies.

30. The method of claim 1 wherein the Sjögren's syndrome is secondary Sjögren's syndrome.

31. An article of manufacture comprising:
a. a container comprising a CD20 antibody;
b. a container comprising an anti-malarial agent; and
c. a package insert with instructions for treating Sjögren's syndrome in a patient, wherein the instructions indicate that amounts of the antibody and anti-malarial agent are administered to the patient that are effective to provide at least about 30% improvement over baseline in two or more of dryness, fatigue, and joint pain on a Visual Analogue Scale.

32. The article of claim 31 further comprising a container comprising a third medicament, wherein the CD20 antibody is a first medicament and the anti-malarial agent is a second medicament, further comprising instructions on the package insert for treating the patient with the third medicament.

33. The article of claim 32 wherein the third medicament is a chemotherapeutic agent, an immunosuppressive agent, a cytotoxic agent, an integrin antagonist, a cytokine antagonist, or a hormone.

34. The article of claim 32 wherein the third medicament is a steroid.

35. A method of treating Sjögren's syndrome in a subject comprising administering an effective amount of a CD20 antibody to the subject to provide an initial antibody exposure followed by a second antibody exposure, wherein the second exposure is not provided until from about 16 to 54 weeks from the initial exposure.

36. The method of claim 35 wherein the second exposure is not provided until from about 20 to 30 weeks from the initial exposure.

37. The method of claim 35 wherein the second exposure is not provided until from about 46 to 54 weeks from the initial exposure.

38. The method of claim 35 wherein each of the initial and second antibody exposures is provided in amounts of about 0.5 to 4 grams.

39. The method of claim 35 wherein each of the initial and second antibody exposures is provided in amounts of about 1.5 to 3.5 grams.

40. The method of claim 35 wherein each of the initial and second antibody exposures is provided in amounts of about 1.5 to 2.5 grams.

41. The method of claim 35 additionally comprising administering to the subject an effective amount of the CD20 antibody to provide a third antibody exposure, wherein the third exposure is not provided until from about 46 to 60 weeks from the initial exposure.

42. The method of claim 41 wherein the third antibody exposure is provided in an amount of about 0.5 to 4 grams.

43. The method of claim 41 wherein the third antibody exposure is provided in an amount of about 1.5 to 3.5 grams.

44. The method of claim 41 wherein the third antibody exposure is provided in an amount of about 1.5 to 2.5 grams.

45. The method of claim 41 wherein the third exposure is not provided until from about 46 to 55 weeks from the initial exposure.

46. The method of claim 41 wherein no further antibody exposure is provided until at least about 70-75 weeks from the initial exposure.

47. The method of claim 46 wherein no further antibody exposure is provided until about 74 to 80 weeks from the initial exposure.

48. The method of claim 35 wherein one or more of the antibody exposures is provided to the subject as a single dose of antibody.

49. The method of claim 48 wherein each antibody exposure is provided to the subject as a single dose of antibody.

50. The method of claim 35 wherein one or more of the antibody exposures is provided to the subject as separate doses of the antibody.

51. The method of claim 50 wherein each antibody exposure is provided as separate doses of the antibody.

52. The method of claim 50 wherein the separate doses are from about 2 to 4 doses.

53. The method of claim 50 wherein the separate doses are from about 2 to 3 doses.

54. The method of claim 52 wherein the separate doses constitute a first and second dose.

55. The method of claim 52 wherein the separate doses constitute a first, second, and third dose.

56. The method of claim 50 wherein a later dose is administered from about 1 to 20 days from the time the previous dose was administered.

57. The method of claim 50 wherein a later dose is administered from about 6 to 16 days from the time the previous dose was administered.

58. The method of claim 50 wherein a later dose is administered from about 14 to 16 days from the time the previous dose was administered.

59. The method of claim 50 wherein the separate doses are administered within a total period of between about 1 day and 4 weeks.

60. The method of claim 50 wherein the separate doses are administered within a total period of between about 1 and 25 days.

61. The method of claim 50 wherein the separate doses are administered about weekly, with the second dose being administered about one week from the first dose and any third or later dose being administered about one week from the previous dose.

62. The method of claim 50 wherein each separate dose of antibody is about 0.5 to 1.5 grams.

63. The method of claim 50 wherein each separate dose of antibody is about 0.75 to 1.3 grams.

64. The method of claim 35 wherein 4 to 20 antibody exposures are administered to the subject.

65. The method of claim 35 wherein a second medicament is administered in an effective amount with an antibody exposure, wherein the CD20 antibody is a first medicament.

66. The method of claim 65 wherein the second medicament is administered with the initial exposure.

67. The method of claim 65 wherein the second medicament is administered with the initial and second exposures.

68. The method of claim 65 wherein the second medicament is administered with all exposures.

69. The method of claim 65 wherein the second medicament is a chemotherapeutic agent, an immunosuppressive agent, a disease-modifying anti-rheumatic drug (DMARD), a cytotoxic agent, an integrin antagonist, a nonsteroidal antiinflammatory drug (NSAID), a cytokine antagonist, a secretory agonist for dry mouth or dry eye, or a hormone.

70. The method of claim 65 wherein the second medicament is an anti-malarial agent.

71. The method of claim 70 wherein the anti-malarial agent is hydroxychloroquine or chloroquine.

72. The method of claim 71 wherein the anti-malarial agent is hydroxychloroquine.

73. The method of claim 70 wherein the second medicament further comprises another medicament.

74. The method of claim 65 wherein the second medicament comprises a steroid, a secretory agonist for dry mouth or dry eye, a nonsteroidal antiinflammatory drug (NSAID), or an immunosuppressive agent.

75. The method of claim 65 wherein the second medicament comprises a steroid.

76. The method of claim 75 wherein the steroid is a corticosteroid.

77. The method of claim 75 wherein the steroid is prednisone, methylprednisolone, hydrocortisone, or dexamethasone.

78. The method of claim 75 wherein the steroid is administered in lower amounts than are used if the CD20 antibody is not administered to a subject treated with steroid.

79. The method of claim 65 wherein the second medicament comprises a secretory agonist for dry mouth or dry eye.

80. The method of claim 79 wherein the secretory agonist is pilocarpine hydrochloride, cevimeline, bromhexine, cyclosporine ophthalmic emulsion, lubricant eye drops, cysteamine eye drops, diquafosol, or pharmaceutical salts thereof.

81. The method of claim 65 wherein the second medicament comprises a nonsteroidal antiinflammatory drug (NSAID).

82. The method of claim 81 wherein the NSAID is aspirin, naproxen, ibuprofen, indomethacin, or tolmetin.

83. The method of claim 65 wherein the second medicament comprises an immunosuppressive agent.

84. The method of claim 83 wherein the immunosuppressive agent is cyclophosphamide, chlorambucil, azathioprine, or methotrexate.

85. The method of claim 65 wherein the second medicament is administered with the initial exposure.

86. The method of claim 85 wherein the second medicament is not administered with the second exposure, or is administered in lower amounts than are used with the initial exposure.

87. The method of claim 35 wherein about 2-3 grams of the CD20 antibody is administered as the initial exposure.

88. The method of claim 87 wherein about 1 gram of the CD20 antibody is administered weekly for about three weeks as the initial exposure.

89. The method of claim 87 wherein the second exposure is at about six months from the initial exposure and is administered in an amount of about 2 grams.

90. The method of claim 87 wherein the second exposure is at about six months from the initial exposure and is administered as about 1 gram of the antibody followed in about two weeks by another about 1 gram of the antibody.

91. The method of claim 87 wherein about 1 gram of the CD20 antibody is administered followed in about two weeks by another about 1 gram of the antibody as the initial exposure.

92. The method of claim 91 wherein the second exposure is at about six months from the initial exposure and is administered in an amount of about 2 grams.

93. The method of claim 91 wherein the second exposure is at about six months from the initial exposure and is administered as about 1 gram of the antibody followed in about two weeks by another about 1 gram of the antibody.

94. The method of claim 87 wherein an anti-malarial agent is administered to the subject before or with the initial exposure.

95. The method of claim 94 further comprising administering a steroid to the subject.

96. The method of claim 95 wherein the steroid is not administered with the second exposure or is administered with the second exposure but in lower amounts than are used with the initial exposure.

97. The method of claim 95 wherein the steroid is not administered with third or later exposures.

98. The method of claim 35 wherein the subject has never been previously treated with a CD20 antibody.

99. The method of claim 35 wherein the antibody is a naked antibody.

100. The method of claim 35 wherein the antibody is conjugated with another molecule.

101. The method of claim 100 wherein the other molecule is a cytotoxic agent.

102. The method of claim 35 wherein the antibody is administered intravenously.

103. The method of claim 102 wherein the antibody is administered intravenously for each antibody exposure.

104. The method of claim 35 wherein the antibody is administered subcutaneously.

105. The method of claim 104 wherein the antibody is administered subcutaneously for each antibody exposure.

106. The method of claim 35 wherein no other medicament than the CD20 antibody is administered to the subject to treat the Sjögren's syndrome.

107. The method of claim 35 wherein the antibody is rituximab.

108. The method of claim 35 wherein the antibody is humanized 2H7 comprising the variable domain sequences in SEQ ID Nos. 2 and 8.

109. The method of claim 35 wherein the antibody is humanized 2H7 comprising the variable domain sequences in SEQ ID NOS:23 and 24.

110. The method of claim 35 wherein the subject has an elevated level of anti-nuclear antibodies (ANA), anti-rheumatoid factor (RF) antibodies, antibodies directed against Sjögren's-associated antigen A or B (SS-A or SS-B), antibodies directed against centromere protein B (CENP B) or centromere protein C (CENP C), an autoantibody to ICA69, or a combination of two or more of such antibodies.

111. The method of claim 110 wherein the antibodies directed against SS-A and SS-B are anti-Ro/SS-A antibodies, anti-La/SS-A antibodies, anti-La/SS-B antibodies, or anti-Ro/SS-B antibodies.

112. The method of claim 35 wherein the Sjögren's syndrome is secondary Sjögren's syndrome.

113. An article of manufacture comprising:
a. a container comprising a CD20 antibody; and
b. a package insert with instructions for treating Sjögren's syndrome in a subject, wherein the instructions indicate that an amount of the antibody is administered to the subject that is effective to provide an initial antibody exposure followed by a second antibody exposure, wherein the second exposure is not provided until from about 16 to 54 weeks from the initial exposure.

114. The article of claim 113 wherein each of the initial and second antibody exposures is provided in an amount of 0.5 to 4 grams.

115. The article of claim 113 wherein each of the antibody exposures is provided to the subject as about 1 to 4 doses.

116. The article of claim 113 wherein each of the antibody exposures is provided to the subject as a single dose or as two or three separate doses of antibody.

117. The article of claim 113 further comprising a container comprising a second medicament, wherein the CD20 antibody is a first medicament, and further comprising instructions on the package insert for treating the subject with the second medicament.

118. The article of claim 117 wherein the second medicament is a chemotherapeutic agent, an immunosuppressive agent, a cytotoxic agent, an integrin antagonist, a cytokine antagonist, or a hormone.

119. The article of claim 117 wherein the second medicament is an anti-malarial agent.

120. The article of claim 117 further comprising a container comprising a third medicament, further comprising instructions on the package insert for treating the subject with the third medicament.

121. The article of claim 120 wherein the third medicament is a steroid.


SEQUENCE LISTING (36 sequences)

SEQ ID NO: 1 - protein, 107 aa, Mus musculus
QIVLSQSPAILSASPGEKVTMTCRASSSVSYMHWYQQKPGSSPKPWIYAPSNLASGVPARFSGSGSGTSYSLTISRVEAEDAATYYCQQWSFNPPTFGAGTKLELKR

SEQ ID NO: 2 - protein, 107 aa, Artificial sequence
  Sequence is synthesized
DIQMTQSPSSLSASVGDRVTITCRASSSVSYMHWYQQKPGKAPKPLIYAPSNLASGVPSRFSGSGSGTDFTLTISSLQPEDFATYYCQQWSFNPPTFGQGTKVEIKR

SEQ ID NO: 3 - protein, 108 aa, Homo sapiens
DIQMTQSPSSLSASVGDRVTITCRASQSISNYLAWYQQKPGKAPKLLIYAASSLESGVPSRFSGSGSGTDFTLTISSLQPEDFATYYCQQYNSLPWTFGQGTKVEIKR

SEQ ID NO: 4 - protein, 26 aa, Artificial sequence
  Sequence is synthesized
RASSSVSYMHAPSNLASQQWSFNPPT

SEQ ID NO: 5 - protein, 26 aa, Artificial sequence
  Sequence is synthesized
RASSSVSYMHAPSNLASQQWSFNPPT

SEQ ID NO: 6 - protein, 27 aa, Artificial sequence
  Sequence is synthesized
RASQSISNYLAAASSLESQQYNSLPWT

SEQ ID NO: 7 - protein, 122 aa, Mus musculus
QAYLQQSGAELVRPGASVKMSCKASGYTFTSYNMHWVKQTPRQGLEWIGAIYPGNGDTSYNQKFKGKATLTVDKSSSTAYMQLSSLTSEDSAVYFCARVVYYSNSYWYFDVWGTGTTVTVSS

SEQ ID NO: 8 - protein, 122 aa, Artificial sequence [sequence omitted]

SEQ ID NO: 9 - protein, 119 aa, Homo sapiens
EVQLVESGGGLVQPGGSLRLSCAASGFTFSSYAMSWVRQAPGKGLEWVAVISGDGGSTYYADSVKGRFTISRDNSKNTLTLQMNSLRAEDTAVYYCARGRVGYSLYDYWGQGTLVTVSS

SEQ ID NO: 10 - protein, 40 aa, Artificial sequence
  Sequence is synthesized
GYTFTSYNMHAIYPGNGDTSYNQKFKGVVYYSNSYWYFDV

SEQ ID NO: 11 - protein, 40 aa, Artificial sequence; identical to SEQ ID NO: 10
  Sequence is synthesized

SEQ ID NO: 12 - protein, 37 aa, Artificial sequence
  Sequence is synthesized
GFTFSSYAMSVISGDGGSTYYADSVKGGRVGYSLYDY

SEQ ID NO: 13 - protein, 213 aa, Artificial sequence
  Sequence is synthesized
DIQMTQSPSSLSASVGDRVTITCRASSSVSYMHWYQQKPGKAPKPLIYAPSNLASGVPSRFSGSGSGTDFTLTISSLQPEDFATYYCQQWSFNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 452 aa, Artificial sequence
  Sequence is synthesized
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 452 aa, Artificial sequence
  Sequence is synthesized
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 285 aa, Homo sapiens
MDDSTEREQSRLTSCLKKREEMKLKECVSILPRKESPSVRSSKDGKLLAATLLLALLSCCLTVVSFYQVAALQGDLASLRAELQGHHAEKLPAGAGAPKAGLEEAPAVTAGLKIFEPPAPGEGNSSQNSRNKRAVQGPEETVTQDCLQLIADSETPTIQKGSYTFVPWLLSFKRGSALEEKENKILVKETGYFFIYGQVLYTDKTYAMGHLIQRKKVHVFGDELSLVTLFRCIQNMPETLPNNSCYSAGIAKLEEGDELQLAIPRENAQISLDGDVTFFGALKLL

SEQ ID NO: 17 - protein, 309 aa, Mus musculus
MDESAKTLPPPCLCFCSEKGEDMKVGYDPITPQKEEGAWFGICRDGRLLAATLLLALLSSSFTAMSLYQLAALQADLMNLRMELQSYRGSATPAAAGAPELTAGVKLLTPAAPRPHNSSRGHRNRRAFQGPEETEQDVDLSAPPAPCLPGCRHSQHDDNGMNLRNIIQDCLQLIADSDTPTIRKGTYTFVPWLLSFKRGNALEEKENKIVVRQTGYFFIYSQVLYTDPIFAMGHVIQRKKVHVFGDELSLVTLFRCIQNMPKTLPNNSCYSAGIARLEEGDEIQLAIPRENAQISRNGDDTFFGALKLL

SEQ ID NO: 18 - protein, 17 aa, Artificial sequence
  Sequence is synthesized
  X 1, 3, 5, 7-12, 14, 15, 17: X = any amino acid except cysteine
  unsure 7-12: unknown amino acid
  X 16: X = an amino acid selected fromthe group consisting of L, F, I and V
XCXDXLXXXXXXCXXXX

SEQ ID NO: 19 - protein, 17 aa, Artificial sequence
  Sequence is synthesized
ECFDLLVRAWVPCSVLK

SEQ ID NO: 20 - protein, 17 aa, Artificial sequence
  Sequence is synthesized
ECFDLLVRHWVPCGLLR

SEQ ID NO: 21 - protein, 17 aa, Artificial sequence
  Sequence is synthesized
ECFDLLVRRWVPCEMLG

SEQ ID NO: 22 - protein, 17 aa, Artificial sequence
  Sequence is synthesized
ECFDLLVRSWVPCHMLR

SEQ ID NO: 23 - protein, 17 aa, Artificial sequence [sequence omitted]

SEQ ID NO: 24 - protein, 16 aa, Artificial sequence
  Sequence is synthesized
QCFDRLNAWVPCSVLK

SEQ ID NO: 25 - protein, 17 aa, Artificial sequence
  Sequence is synthesized
  Unsure 1, 3, 5, 8, 9, 14, 15, 17: any amino acid, except cysteine
XCXDXLVXXWVPCXXLX

SEQ ID NO: 26 - protein, 184 aa, Homo sapiens
MRRGPRSLRGRDAPAPTPCVPAECFDLLVRHCVACGLLRTPRPKPAGASSPAPRTALQPQESVGAGAGEAALPLPGLLFGAPALLGLALVLALVLVGLVSWRRRQRRLRGASSAEAPDGDKDAPEPLDKVIILSPGISDATAPAWPPPGEDPGTTPPGHSVPVPATELGSTELVTTKTAGPEQQ

SEQ ID NO: 27 - protein, 26 aa, Homo sapien
TPCVPAECFDLLVRHCVACGLLRTPR

SEQ ID NO: 28 - protein, 213 aa, Artificial sequence
  Sequence is synthesized
DIQMTQSPSSLSASVGDRVTITCRASSSVSYLHWYQQKPGKAPKPLIYAPSNLASGVPSRFSGSGSGTDFTLTISSLQPEDFATYYCQQWAFNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 451 aa, Artificial sequence
  Sequence is synthesized
EVQLVESGGGLVQPGGSLRLSCAASGYTFTSYNMHWVRQAPGKGLEWVGIYPGNGATSYNQKFKGRFTISVDKSKNTLYLQMNSLRAEDTAVYYCARVVYYSYRYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNAALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 10 aa, Artificial sequence
  sequence is synthesized
  Unsure 9: wherein X is M or L
RASSSVSYXH

SEQ ID NO: 31 - protein, 9 aa, Artificial sequence
  Sequence is synthesized
  Unsure 4: wherein X is S or A
QQWXFNPPT

SEQ ID NO: 32 - protein, 17 aa, Artificial sequence
  Sequence is synthesized
  Unsure 8: wherein X is D or A
AIYPGNGXTSYNQKFKG

SEQ ID NO: 33 - protein, 13 aa, Artificial sequence
  Sequence is synthesized
  Unsure 6: wherein X is N, A, Y, W, or D
  Unsure 7: wherein X is S or R
VVYYSXXYWYFDV

SEQ ID NO: 34 - protein, 451 aa, Artificial sequence
  Sequence is synthesized
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSYRYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNAALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 35 - protein, 107 aa, Artificial sequence
  Sequence is synthesized
DIQMTQSPSSLSASVGDRVTITCRASSSVSYLHWYQQKPGKAPKPLIYAPSNLASGVPSRFSGSGSGTDFTLTISSLQPEDFATYYCQQWAFNPPTFGQGTKVEIKR

SEQ ID NO: 36 - protein, 122 aa, Artificial sequence
  Sequence is synthesized
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSYRYWYFDVWGQGTLVTVSS